SEQUENCE LISTING (144 sequences)

SEQ ID NO: 1 - protein, 508 aa, Homo sapiens
  REGION 1..508: note = Description of sequence: Naturally Occurring Receptor; name: Erythropoietin receptor
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 2 - protein, 918 aa, Homo sapiens [sequence omitted]

SEQ ID NO: 3 - protein, 622 aa, Homo sapiens
  REGION 1..622: note = Description of sequence: Naturally Occurring Receptor; name: prolactin receptor isoform X2
MKENVASATVFTLLLFLNTCLLNGQLPPGKPEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEDRKPYLWIKWSPPTLIDLKTGWFTLLYEIRLKPEKAAEWEIHFAGQQTEFKILSLHPGQKYLVQVRCKPDHGYWSAWSPATFIQIPSDFTMNDTTVWISVAVLSAVICLIIVWAVALKGYSMVTCIFPPVPGPKIKGFDAHLLEKGKSEELLSALGCQDFPPTSDYEDLLVEYLEVDDSEDQHLMSVHSKEHPSQGMKPTYLDPDTDSGRGSCDSPSLLSEKCEEPQANPSTFYDPEVIEKPENPETTHTWDPQCISMEGKIPYFHAGGSKCSTWPLPQPSQHNPRSSYHNITDVCELAVGPAGAPATLLNEAGKDALKSSQTIKSREEGKATQQREVESFHSETDQDTPWLLPQEKTPFGSAKPLDYVEIHKVNKDGALSLLPKQRENSGKPKKPGTPENNKEYAKVSGVMDNNILVLVPDPHAKNVACFEESAKEAPPSLEQNQAEKALANFTATSSKCRLQLGGLDYLDPACFTHSFH

SEQ ID NO: 4 - protein, 638 aa, Homo sapiens
  REGION 1..638: note = Description of sequence: Naturally Occurring Receptor; name: growth hormone receptor isoform 1 precursor
MDLWQLLLTLALAGSSDAFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFYFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 5 - protein, 863 aa, Homo sapiens
  REGION 1..863: note = Description of sequence: Naturally Occurring Receptor; name: granulocyte colony-stimulating factor receptor isoform X1
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEELPGPRQGQWLGQTSEMSRALTPHPCVQDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 6 - protein, 635 aa, Homo sapiens
  REGION 1..635: note = Description of sequence: Naturally Occurring Receptor; name: thrombopoietin receptor precursor
MPSWALFMVTSCLLLAPQNLAQVSSQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 7 - protein, 105 aa, synthetic construct
  REGION 1..105: note = Description of sequence: Transmembrane Domain; name: TPOR/MPLR(478-582)
SDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 8 - protein, 105 aa, synthetic construct
  REGION 1..105: note = Description of sequence: Transmembrane Domain; name: TPOR/MPLR(478-582; H499LS505N)
SDPTRVETATETAWISLVTALLLVLGLNAVLGLLLLRWQFPAHYRRLRHALWPSLPDLHRVLGQYLRDTAALSPPKATVSDTCEEVEPSLLEILPKSSERTPLPL

SEQ ID NO: 9 - protein, 105 aa, synthetic construct
  REGION 1..105: note = Description of sequence: Transmembrane Domain; name: TPOR/MPLR(478-582;S505N)
SDPTRVETATETAWISLVTALHLVLGLNAVLGLLLLRWQFPAHYRRLRHALWPSLPDLHRVLGQYLRDTAALSPPKATVSDTCEEVEPSLLEILPKSSERTPLPL

SEQ ID NO: 10 - protein, 105 aa, synthetic construct
  REGION 1..105: note = Description of sequence: Transmembrane Domain; name: TPOR/MPLR(478-582;H499LW515K)
SDPTRVETATETAWISLVTALLLVLGLSAVLGLLLLRKQFPAHYRRLRHALWPSLPDLHRVLGQYLRDTAALSPPKATVSDTCEEVEPSLLEILPKSSERTPLPL

SEQ ID NO: 11 - protein, 105 aa, synthetic construct
  REGION 1..105: note = Description of sequence: Transmembrane Domain; name: TPOR/MPLR(478-582;W515K)
SDPTRVETATETAWISLVTALHLVLGLSAVLGLLLLRKQFPAHYRRLRHALWPSLPDLHRVLGQYLRDTAALSPPKATVSDTCEEVEPSLLEILPKSSERTPLPL

SEQ ID NO: 12 - protein, 105 aa, synthetic construct
  REGION 1..105: note = Description of sequence: Transmembrane Domain; name: TPOR/MPLR(478-582;H499LS505NW515K)
SDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPL

SEQ ID NO: 13 - protein, 105 aa, synthetic construct
  REGION 1..105: note = Description of sequence: Transmembrane Domain; name: TPOR/MPLR(478-582;S505NW515K)
SDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPL

SEQ ID NO: 14 - protein, 105 aa, synthetic construct
  REGION 1..105: note = Description of sequence: Transmembrane Domain; name: TPOR/MPLR(478-582;H499LG509N)
SDPTRVETATETAWISLVTALLLVLGLSAVLNLLLLRWQFPAHYRRLRHALWPSLPDLHRVLGQYLRDTAALSPPKATVSDTCEEVEPSLLEILPKSSERTPLPL

SEQ ID NO: 15 - protein, 97 aa, synthetic construct
  REGION 1..97: note = Description of sequence: Transmembrane and JAK2 binding domain; name: GCSFR(614-710)
LTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWE

SEQ ID NO: 16 - protein, 92 aa, synthetic construct
  REGION 1..92: note = Description of sequence: Transmembrane and JAK2 binding domain; name: GP130(609-700)
TTPKFAQGEIEAIVVPVCLAFLLTTLLGVLFCFNKRDLIKKHIWPNVPDPSKSHIAQWSPHTPPRHNFNSKDQMYSDGNFTDVSVVEIEAND

SEQ ID NO: 17 - protein, 105 aa, synthetic construct; identical to SEQ ID NO: 7
  REGION 1..105: note = Description of sequence: Transmembrane and JAK2 binding domain; name: TPOR/MPLR(478-582)

SEQ ID NO: 18 - protein, 104 aa, synthetic construct
  REGION 1..104: note = Description of sequence: Transmembrane and JAK2 binding domain; name: TPOR/MPLR(N-1)
SDPTRVETATETWISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 19 - protein, 103 aa, synthetic construct
  REGION 1..103: note = Description of sequence: Transmembrane and JAK2 binding domain; name: TPOR/MPLR(N-2)
SDPTRVETATETISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 20 - protein, 104 aa, synthetic construct
  REGION 1..104: note = Description of sequence: Transmembrane and JAK2 binding domain; name: TPOR/MPLR(N-2+1)
SDPTRVETATETLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 21 - protein, 102 aa, synthetic construct
  REGION 1..102: note = Description of sequence: Transmembrane and JAK2 binding domain; name: TPOR/MPLR(N-3)
SDPTRVETATETSLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 22 - protein, 101 aa, synthetic construct
  REGION 1..101: note = Description of sequence: Transmembrane and JAK2 binding domain; name: TPOR/MPLR(N-4)
SDPTRVETATETLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 23 - protein, 102 aa, synthetic construct
  REGION 1..102: note = Description of sequence: Transmembrane and JAK2 binding domain; name: TPOR/MPLR(N-4+1)
SDPTRVETATETILVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 24 - protein, 100 aa, synthetic construct
  REGION 1..100: note = Description of sequence: Transmembrane and JAK2 binding domain; name: TPOR/MPLR(N-5)
SDPTRVETATETVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 25 - protein, 99 aa, synthetic construct
  REGION 1..99: note = Description of sequence: Transmembrane and JAK2 binding domain; name: TPOR/MPLR(N-6)
SDPTRVETATETTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 26 - protein, 98 aa, synthetic construct
  REGION 1..98: note = Description of sequence: Transmembrane and JAK2 binding domain; name: TPOR/MPLR(N-7)
SDPTRVETATETALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 27 - protein, 97 aa, synthetic construct
  REGION 1..97: note = Description of sequence: Transmembrane and JAK2 binding domain; name: TPOR/MPLR(N-8)
SDPTRVETATETLHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 28 - protein, 96 aa, synthetic construct
  REGION 1..96: note = Description of sequence: Transmembrane and JAK2 binding domain; name: TPOR/MPLR(N-9)
SDPTRVETATETHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 29 - protein, 95 aa, synthetic construct
  REGION 1..95: note = Description of sequence: Transmembrane and JAK2 binding domain; name: TPOR/MPLR(N-10)
SDPTRVETATETLVLGLSAVLGLLLLRWQFPAHYRRLRHALWPSLPDLHRVLGQYLRDTAALSPPKATVSDTCEEVEPSLLEILPKSSERTPLPL

SEQ ID NO: 30 - protein, 94 aa, synthetic construct
  REGION 1..94: note = Description of sequence: Transmembrane and JAK2 binding domain; name: TPOR/MPLR(N-11)
SDPTRVETATETVLGLSAVLGLLLLRWQFPAHYRRLRHALWPSLPDLHRVLGQYLRDTAALSPPKATVSDTCEEVEPSLLEILPKSSERTPLPL

SEQ ID NO: 31 - protein, 93 aa, synthetic construct
  REGION 1..93: note = Description of sequence: Transmembrane and JAK2 binding domain; name: TPOR/MPLR(N-12)
SDPTRVETATETLGLSAVLGLLLLRWQFPAHYRRLRHALWPSLPDLHRVLGQYLRDTAALSPPKATVSDTCEEVEPSLLEILPKSSERTPLPL

SEQ ID NO: 32 - protein, 92 aa, synthetic construct
  REGION 1..92: note = Description of sequence: Transmembrane and JAK2 binding domain; name: TPOR/MPLR(N-13)
SDPTRVETATETGLSAVLGLLLLRWQFPAHYRRLRHALWPSLPDLHRVLGQYLRDTAALSPPKATVSDTCEEVEPSLLEILPKSSERTPLPL

SEQ ID NO: 33 - protein, 91 aa, synthetic construct
  REGION 1..91: note = Description of sequence: Transmembrane and JAK2 binding domain; name: TPOR/MPLR(N-14)
SDPTRVETATETLSAVLGLLLLRWQFPAHYRRLRHALWPSLPDLHRVLGQYLRDTAALSPPKATVSDTCEEVEPSLLEILPKSSERTPLPL

SEQ ID NO: 34 - protein, 90 aa, synthetic construct
  REGION 1..90: note = Description of sequence: Transmembrane and JAK2 binding domain; name: TPOR/MPLR(N-15)
SDPTRVETATETSAVLGLLLLRWQFPAHYRRLRHALWPSLPDLHRVLGQYLRDTAALSPPKATVSDTCEEVEPSLLEILPKSSERTPLPL

SEQ ID NO: 35 - protein, 89 aa, synthetic construct
  REGION 1..89: note = Description of sequence: Transmembrane and JAK2 binding domain; name: TPOR/MPLR(N-16)
SDPTRVETATETAVLGLLLLRWQFPAHYRRLRHALWPSLPDLHRVLGQYLRDTAALSPPKATVSDTCEEVEPSLLEILPKSSERTPLPL

SEQ ID NO: 36 - protein, 88 aa, synthetic construct
  REGION 1..88: note = Description of sequence: Transmembrane and JAK2 binding domain; name: TPOR/MPLR(N-17)
SDPTRVETATETVLGLLLLRWQFPAHYRRLRHALWPSLPDLHRVLGQYLRDTAALSPPKATVSDTCEEVEPSLLEILPKSSERTPLPL

SEQ ID NO: 37 - protein, 87 aa, synthetic construct
  REGION 1..87: note = Description of sequence: Transmembrane and JAK2 binding domain; name: TPOR/MPLR(N-18)
SDPTRVETATETLGLLLLRWQFPAHYRRLRHALWPSLPDLHRVLGQYLRDTAALSPPKATVSDTCEEVEPSLLEILPKSSERTPLPL

SEQ ID NO: 38 - protein, 106 aa, synthetic construct
  REGION 1..106: note = Description of sequence: Transmembrane and JAK2 binding domain; name: TPOR/MPLR(N+1)
SDPTRVETATETAWLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 39 - protein, 107 aa, synthetic construct
  REGION 1..107: note = Description of sequence: Transmembrane and JAK2 binding domain; name: TPOR/MPLR(N+2)
SDPTRVETATETAWVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 40 - protein, 108 aa, synthetic construct
  REGION 1..108: note = Description of sequence: Transmembrane and JAK2 binding domain; name: TPOR/MPLR(N+3)
SDPTRVETATETAWLVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 41 - protein, 109 aa, synthetic construct
  REGION 1..109: note = Description of sequence: Transmembrane and JAK2 binding domain; name: TPOR/MPLR(N+4)
SDPTRVETATETAWILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 42 - protein, 110 aa, synthetic construct
  REGION 1..110: note = Description of sequence: Transmembrane and JAK2 binding domain; name: TPOR/MPLR(N+5)
SDPTRVETATETAWLILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 43 - protein, 111 aa, synthetic construct
  REGION 1..111: note = Description of sequence: Transmembrane and JAK2 binding domain; name: TPOR/MPLR(N+6)
SDPTRVETATETAWLLILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 44 - protein, 112 aa, synthetic construct
  REGION 1..112: note = Description of sequence: Transmembrane and JAK2 binding domain; name: TPOR/MPLR(N+7)
SDPTRVETATETAWVLLILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 45 - protein, 113 aa, synthetic construct
  REGION 1..113: note = Description of sequence: Transmembrane and JAK2 binding domain; name: TPOR/MPLR(N+8)
SDPTRVETATETAWLVLLILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 46 - protein, 144 aa, synthetic construct
  REGION 1..144: note = Description of sequence: Cytotail sequence; name: IL7R(316-459)
ARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 47 - protein, 219 aa, synthetic construct
  REGION 1..219: note = Description of sequence: Cytotail sequence; name: IL2Rb(333-551)
VTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 48 - protein, 50 aa, synthetic construct
  REGION 1..50: note = Description of sequence: Cytotail sequence; name: IFNAR1(508-557)
ISTIATVEETNQTDEDHKKYSSQTSQDSGNYSNEDESESKTSEELQQDFV

SEQ ID NO: 49 - protein, 206 aa, synthetic construct
  REGION 1..206: note = Description of sequence: Cytotail sequence; name: IFNAR2(310-515)
KKKVWDYNYDDESDSDTEAAPRTSGGGYTMHGLTVRPLGQASATSTESQLIDPESEEEPDLPEVDVELPTMPKDSPQQLELLSGPCERRKSPLQDPFPEEDYSSTEGSGGRITFNVDLNSVFLRVLDDEDSDDLEAPLMLSSHLEEMVDPEDPDNVQSNHLLASGEGTQPTFPSPSSEGLWSEDAPSDQSDTSESDVDLGDGYIMR

SEQ ID NO: 50 - protein, 256 aa, synthetic construct
  REGION 1..256: note = Description of sequence: Cytotail sequence; name: IFNAR1/2(IFNAR1 residues 508-557-IFNAR2 residues 310-515)
ISTIATVEETNQTDEDHKKYSSQTSQDSGNYSNEDESESKTSEELQQDFVKKKVWDYNYDDESDSDTEAAPRTSGGGYTMHGLTVRPLGQASATSTESQLIDPESEEEPDLPEVDVELPTMPKDSPQQLELLSGPCERRKSPLQDPFPEEDYSSTEGSGGRITFNVDLNSVFLRVLDDEDSDDLEAPLMLSSHLEEMVDPEDPDNVQSNHLLASGEGTQPTFPSPSSEGLWSEDAPSDQSDTSESDVDLGDGYIMR

SEQ ID NO: 51 - protein, 221 aa, synthetic construct
  REGION 1..221: note = Description of sequence: Cytotail sequence; name: IFNLR1(300-520)
RGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 52 - protein, 35 aa, synthetic construct
  REGION 1..35: note = Description of sequence: Cytotail sequence; name: IL2RG (335-369)
IPPKGGALGEGPGASPCNQHSPYWAPPCYTLKPET

SEQ ID NO: 53 - protein, 166 aa, synthetic construct
  REGION 1..166: note = Description of sequence: Cytotail sequence; name: IL9R(356-521)
TALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 54 - protein, 217 aa, synthetic construct
  REGION 1..217: note = Description of sequence: Cytotail sequence; name: IL21R(322-538)
PRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 55 - protein, 286 aa, synthetic construct
  REGION 1..286: note = Description of sequence: Cytotail sequence; name: GHR(353-638)
PDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 56 - protein, 170 aa, synthetic construct
  REGION 1..170: note = Description of sequence: Cytotail sequence; name: EpoR(339-508)
WGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 57 - protein, 203 aa, synthetic construct
  REGION 1..203: note = Description of sequence: Cytotail sequence; name: murine IL2Rb(337-539)
AVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLI

SEQ ID NO: 58 - protein, 144 aa, synthetic construct
  REGION 1..144: note = Description of sequence: Cytotail sequence; name: murine IL7Ra(316-459)
ARDEVESFLPNDLPAQPEELETQGHRAAVHSANRSPETSVSPPETVRRESPLRCLARNLSTCNAPPLLSSRSPDYRDGDRNRPPVYQDLLPNSGNTNVPVPVPQPLPFQSGILIPVSQRQPISTSSVLNQEEAYVTMSSFYQNK

SEQ ID NO: 59 - protein, 231 aa, synthetic construct
  REGION 1..231: note = Description of sequence: Cytotail sequence; name: EGFR(955-1186)
VIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 60 - protein, 218 aa, synthetic construct
  REGION 1..218: note = Description of sequence: Cytotail sequence; name: EGFR(955-1186;Y974Fd1045-1057)
VIQGDERMHLPSPTDSNFFRALMDEEDMDDVVDADEYLIPQQGFFSSPSTSRTPLLSSLSATSNNSTVACIDRNGLQSCPIKEDSFLQRIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 61 - protein, 54 aa, synthetic construct
  REGION 1..54: note = Description of sequence: Cytotail sequence; name: EGFR(955-1009;Y974F)
VIQGDERMHLPSPTDSNFFRALMDEEDMDDVVDADEYLIPQQGFFSSPSTSRTP

SEQ ID NO: 62 - protein, 54 aa, synthetic construct
  REGION 1..54: note = Description of sequence: Cytotail sequence; name: EGFR(1019-1085)
NNSTVACIDRNGLQSCPIKEDSFLQRIDDTFLPVPEYINQSVPKRPAGSVQNPV

SEQ ID NO: 63 - protein, 54 aa, synthetic construct
  REGION 1..54: note = Description of sequence: Cytotail sequence; name: EGFR(1037-1103;Y1068/1101Fd1045-1057)
KEDSFLQRIDDTFLPVPEFINQSVPKRPAGSVQNPVYHNQPLNPAPSRDPHFQD

SEQ ID NO: 64 - protein, 54 aa, synthetic construct
  REGION 1..54: note = Description of sequence: Cytotail sequence; name: EGFR(1066-1118;Y1068/1086F)
VPEFINQSVPKRPAGSVQNPVFHNQPLNPAPSRDPHYQDPHSTAVGNPEYLNTV

SEQ ID NO: 65 - protein, 54 aa, synthetic construct
  REGION 1..54: note = Description of sequence: Cytotail sequence; name: EGFR(1122-1165)
PEYLNTVQPTCVNSTFDSPAHWAQKGSHQISLDNPDYQQDFFPKEAKPNGIFKG

SEQ ID NO: 66 - protein, 54 aa, synthetic construct
  REGION 1..54: note = Description of sequence: Cytotail sequence; name: EGFR(1133-1186;Y1148F)
WAQKGSHQISLDNPDFQQDFFPKEAKPNGIFKGSTAENAEYLRVAPQSSEFIGA

SEQ ID NO: 67 - protein, 51 aa, synthetic construct
  REGION 1..51: note = Description of sequence: Cytotail sequence; name: IL12Rb2(775-825)
SDPKPENPACPWTVLPAGDLPTHDGYLPSNIDDLPSHEAPLADSLEELEPQ

SEQ ID NO: 68 - protein, 41 aa, synthetic construct
  REGION 1..41: note = Description of sequence: Cytotail sequence; name: IL7Ra(376-416)
ACDAPILSSSRSLDCRESGKNGPHVYQDLLLSLGTTNSTLP

SEQ ID NO: 69 - protein, 36 aa, synthetic construct
  REGION 1..36: note = Description of sequence: Cytotail sequence; name: IL7Ra(424-459)
GILTLNPVAQGQPILTSLGSNQEEAYVTMSSFYQNQ

SEQ ID NO: 70 - protein, 68 aa, synthetic construct
  REGION 1..68: note = Description of sequence: Cytotail sequence; name: IL7Ra(376-416424-459)
ACDAPILSSSRSLDCRESGKNGPHVYQDLLLSLGTTNSTLPQGQPILTSLGSNQEEAYVTMSSFYQNQ

SEQ ID NO: 71 - protein, 36 aa, synthetic construct
  REGION 1..36: note = Description of sequence: Cytotail sequence; name: IL7Ra(424-459;Y456F)
GILTLNPVAQGQPILTSLGSNQEEAYVTMSSFFQNQ

SEQ ID NO: 72 - protein, 68 aa, synthetic construct
  REGION 1..68: note = Description of sequence: Cytotail sequence; name: IL7R(376-416424-459Y456F)
ACDAPILSSSRSLDCRESGKNGPHVYQDLLLSLGTTNSTLPQGQPILTSLGSNQEEAYVTMSSFFQNQ

SEQ ID NO: 73 - protein, 75 aa, synthetic construct
  REGION 1..75: note = Description of sequence: Cytotail sequence; name: IL2Rbsmall(393-433)
DEGVAGAPTGSSPQPLQPLSGEDDAYCTFPSRDDLLLFSPSGQGEFRALNARLPLNTDAYLSLQELQGQDPTHLV

SEQ ID NO: 74 - protein, 34 aa, synthetic construct
  REGION 1..34: note = Description of sequence: Cytotail sequence; name: IL2Rbsmall(518-551)
GQGEFRALNARLPLNTDAYLSLQELQGQDPTHLV

SEQ ID NO: 75 - protein, 82 aa, synthetic construct
  REGION 1..82: note = Description of sequence: Cytotail sequence; name: IL2Rbsmall(339-379393-433)
QQDKVPEPASLSSNHSLTSCFTNQGYFFFHLPDALEIEACQDEGVAGAPTGSSPQPLQPLSGEDDAYCTFPSRDDLLLFSPS

SEQ ID NO: 76 - protein, 75 aa, synthetic construct
  REGION 1..75: note = Description of sequence: Cytotail sequence; name: IL2Rbsmall(339-379518-551)
QQDKVPEPASLSSNHSLTSCFTNQGYFFFHLPDALEIEACQGQGEFRALNARLPLNTDAYLSLQELQGQDPTHLV

SEQ ID NO: 77 - protein, 75 aa, synthetic construct; identical to SEQ ID NO: 73
  REGION 1..75: note = Description of sequence: Cytotail sequence; name: IL2Rbsmall(393-433518-551)

SEQ ID NO: 78 - protein, 116 aa, synthetic construct
  REGION 1..116: note = Description of sequence: Cytotail sequence; name: IL2Rbsmall(339-379393-433518-551)
QQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV

SEQ ID NO: 79 - protein, 43 aa, synthetic construct
  REGION 1..43: note = Description of sequence: Cytotail sequence; name: IFNAR2small(310-352)
KKKVWDYNYDDESDSDTEAAPRTSGGGYTMHGLTVRPLGQASA

SEQ ID NO: 80 - protein, 29 aa, synthetic construct
  REGION 1..29: note = Description of sequence: Cytotail sequence; name: IFNAR2small(486-515)
EGLWSEDAPSDQSDTSESDVDLGDGYIMR

SEQ ID NO: 81 - protein, 72 aa, synthetic construct
  REGION 1..72: note = Description of sequence: Cytotail sequence; name: IFNAR2small(310-352486-515)
KKKVWDYNYDDESDSDTEAAPRTSGGGYTMHGLTVRPLGQASAEGLWSEDAPSDQSDTSESDVDLGDGYIMR

SEQ ID NO: 82 - protein, 156 aa, synthetic construct
  REGION 1..156: note = Description of sequence: Cytotail sequence; name: BLNK(53-208)
ASESPADEEEQWSDDFDSDYENPDEHSDSEMYVMPAEENADDSYEPPPVEQETRPVHPALPFARGEYIDNRSSQRHSPPFSKTLPSKPSWPSEKARLTSTLPALTALQKPQVPPKPKGLLEDEADYVVPVEDNDENYIHPTESSSPPPEKAPMVNR

SEQ ID NO: 83 - protein, 156 aa, synthetic construct
  REGION 1..156: note = Description of sequence: Cytotail sequence; name: BLNK(53-208;Y72F)
ASESPADEEEQWSDDFDSDFENPDEHSDSEMYVMPAEENADDSYEPPPVEQETRPVHPALPFARGEYIDNRSSQRHSPPFSKTLPSKPSWPSEKARLTSTLPALTALQKPQVPPKPKGLLEDEADYVVPVEDNDENYIHPTESSSPPPEKAPMVNR

SEQ ID NO: 84 - protein, 156 aa, synthetic construct
  REGION 1..156: note = Description of sequence: Cytotail sequence; name: BLNK(53-208;Y72FY96F)
ASESPADEEEQWSDDFDSDFENPDEHSDSEMYVMPAEENADDSFEPPPVEQETRPVHPALPFARGEYIDNRSSQRHSPPFSKTLPSKPSWPSEKARLTSTLPALTALQKPQVPPKPKGLLEDEADYVVPVEDNDENYIHPTESSSPPPEKAPMVNR

SEQ ID NO: 85 - protein, 170 aa, synthetic construct; identical to SEQ ID NO: 56
  REGION 1..170: note = Description of sequence: Cytotail sequence; name: EpoR(339-508)

SEQ ID NO: 86 - protein, 149 aa, synthetic construct
  REGION 1..149: note = Description of sequence: Cytotail sequence; name: IL12Rb2(714-862)
VTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 87 - protein, 40 aa, synthetic construct
  REGION 1..40: note = Description of sequence: Cytotail sequence; name: IL12Rb1(622-662)
WDKGERTEPLEKTELPEGAPELALDTELSLEDGDRCKAKM

SEQ ID NO: 88 - protein, 275 aa, synthetic construct
  REGION 1..275: note = Description of sequence: Cytotail sequence; name: IL10R1(304-578)
VSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 89 - protein, 21 aa, synthetic construct
  REGION 1..21: note = Description of sequence: a CD8SS of sequence
MALPVTALLLPLALLLHAARP

SEQ ID NO: 90 - protein, 249 aa, synthetic construct
  REGION 1..249: note = Description of sequence: Receptor; name: TPOR/MPLR(478-582).IL7Ra(316-459)
SDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 91 - protein, 249 aa, synthetic construct
  REGION 1..249: note = Description of sequence: Receptor; name: TPOR/MPLR(478-582; H499LS505N).IL7Ra(316-459)
SDPTRVETATETAWISLVTALLLVLGLNAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 92 - protein, 249 aa, synthetic construct
  REGION 1..249: note = Description of sequence: Receptor; name: TPOR/MPLR(478-582;S505N).IL7Ra(316-459)
SDPTRVETATETAWISLVTALHLVLGLNAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 93 - protein, 249 aa, synthetic construct
  REGION 1..249: note = Description of sequence: Receptor; name: TPOR/MPLR(478-582;H499LW515K).IL7Ra(316-459)
SDPTRVETATETAWISLVTALLLVLGLSAVLGLLLLRKQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 94 - protein, 249 aa, synthetic construct
  REGION 1..249: note = Description of sequence: Receptor; name: TPOR/MPLR(478-582;W515K).IL7Ra(316-459)
SDPTRVETATETAWISLVTALHLVLGLSAVLGLLLLRKQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 95 - protein, 249 aa, synthetic construct
  REGION 1..249: note = Description of sequence: Receptor; name: TPOR/MPLR(478-582;H499LS505NW515K).IL7Ra(316-459)
SDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 96 - protein, 249 aa, synthetic construct
  REGION 1..249: note = Description of sequence: Receptor; name: TPOR/MPLR(478-582;S505NW515K).IL7Ra(316-459)
SDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 97 - protein, 249 aa, synthetic construct
  REGION 1..249: note = Description of sequence: Receptor; name: TPOR/MPLR(478-582;H499LG509N).IL7Ra(316-459)
SDPTRVETATETAWISLVTALLLVLGLSAVLNLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 98 - protein, 249 aa, synthetic construct; identical to SEQ ID NO: 91
  REGION 1..249: note = Description of sequence: Receptor; name: TPOR/MPLR(478-582; H499LS505N).IL7Ra(316-459)

SEQ ID NO: 99 - protein, 11 aa, synthetic construct
  REGION 1..11: note = Description of sequence: Domain; name: V5 epitope tag
IPNPLLGLDST

SEQ ID NO: 100 - protein, 246 aa, synthetic construct
  REGION 1..246: note = Description of sequence: Domain; name: 2173 scFv
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 101 - protein, 69 aa, synthetic construct
  REGION 1..69: note = Description of sequence: Domain; name: CD8 hinge and transmembrane
TTTPAPRPPTPAPTIASQPLSLRPEACRPAAGGAVHTRGLDFACDIYIWAPLAGTCGVLLLSLVITLYC

SEQ ID NO: 102 - protein, 42 aa, synthetic construct
  REGION 1..42: note = Description of sequence: Domain; name: 4-1BB intracellular signaling
KRGRKKLLYIFKQPFMRPVQTTQEEDGCSCRFPEEEEGGCEL

SEQ ID NO: 103 - protein, 112 aa, synthetic construct
  REGION 1..112: note = Description of sequence: Domain; name: CD3z intracellular signaling
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 233 aa, synthetic construct
  REGION 1..233: note = Description of sequence: Domain; name: BFP
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 105 - protein, 22 aa, synthetic construct
  REGION 1..22: note = Description of sequence: Domain; name: P2A
GSGATNFSLLKQAGDVEENPGP

SEQ ID NO: 106 - protein, 219 aa, synthetic construct
  REGION 1..219: note = Description of sequence: Cytotail sequence; name: IL2Rb(333-551 Y381SY384SY387S)
VTQLLLQQDKVPEPASLSSNHSLTSCFTNQGYFFFHLPDALEIEACQVSFTSDPSSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 107 - protein, 275 aa, synthetic construct
  REGION 1..275: note = Description of sequence: Receptor; name: CD8SS-TPOR/MPLR(478-582;S505NW515K). IL12Rb2(714-862)
MALPVTALLLPLALLLHAARPSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 108 - protein, 275 aa, synthetic construct
  REGION 1..275: note = Description of sequence: Receptor; name: CD8SS-TPOR/MPLR(478-582;H499LS505NW515K).IL12Rb2(714-862)
MALPVTALLLPLALLLHAARPSDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 109 - protein, 177 aa, synthetic construct
  REGION 1..177: note = Description of sequence: Receptor; name: CD8SS-TPOR/MPLR(478-582;S505NW515K).IL12Rb2(775-825)
MALPVTALLLPLALLLHAARPSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLSDPKPENPACPWTVLPAGDLPTHDGYLPSNIDDLPSHEAPLADSLEELEPQ

SEQ ID NO: 110 - protein, 177 aa, synthetic construct
  REGION 1..177: note = Description of sequence: Receptor; name: CD8SS-TPOR/MPLR(478-582;H499LS505NW515K). IL12Rb2(775-825)
MALPVTALLLPLALLLHAARPSDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLSDPKPENPACPWTVLPAGDLPTHDGYLPSNIDDLPSHEAPLADSLEELEPQ

SEQ ID NO: 111 - protein, 345 aa, synthetic construct
  REGION 1..345: note = Description of sequence: Receptor; name: CD8SS-TPOR/MPLR(478-582;S505NW515K). IL2Rb(333-551)
MALPVTALLLPLALLLHAARPSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 112 - protein, 345 aa, synthetic construct
  REGION 1..345: note = Description of sequence: Receptor; name: CD8SS-TPOR/MPLR(478-582;H499LS505NW515K). IL2Rb(333-551)
MALPVTALLLPLALLLHAARPSDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 113 - protein, 201 aa, synthetic construct
  REGION 1..201: note = Description of sequence: Receptor; name: CD8SS-TPOR/MPLR(478-582;S505NW515K).IL2Rb(393-433518-551)
MALPVTALLLPLALLLHAARPSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLDEGVAGAPTGSSPQPLQPLSGEDDAYCTFPSRDDLLLFSPSGQGEFRALNARLPLNTDAYLSLQELQGQDPTHLV

SEQ ID NO: 114 - protein, 201 aa, synthetic construct
  REGION 1..201: note = Description of sequence: Receptor; name: CD8SS-TPOR /MPLR(478-582;H499LS505NW515K).IL2Rb(393-433518-551)
MALPVTALLLPLALLLHAARPSDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLDEGVAGAPTGSSPQPLQPLSGEDDAYCTFPSRDDLLLFSPSGQGEFRALNARLPLNTDAYLSLQELQGQDPTHLV

SEQ ID NO: 115 - protein, 242 aa, synthetic construct
  REGION 1..242: note = Description of sequence: Receptor; name: CD8SS-TPOR/MPLR(478-582;S505NW515K). IL2Rb(339-379393-433518-551)
MALPVTALLLPLALLLHAARPSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLQQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV

SEQ ID NO: 116 - protein, 242 aa, synthetic construct
  REGION 1..242: note = Description of sequence: Receptor; name: CD8SS-TPOR /MPLR(478-582;H499LS505NW515K).IL2Rb(339-379393-433518-551 )
MALPVTALLLPLALLLHAARPSDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLQQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV

SEQ ID NO: 117 - protein, 321 aa, synthetic construct
  REGION 1..321: note = Description of sequence: Receptor; name: CD8SS-TPOR /MPLR(478-582;S505NW515K).IL7Ra(316-459).IL12Rb2(775-825)
MALPVTALLLPLALLLHAARPSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 118 - protein, 321 aa, synthetic construct
  REGION 1..321: note = Description of sequence: Receptor; name: CD8SS-TPOR /MPLR(478-582;H499LS505NW515K).IL7Ra(316-459).IL12Rb2(775- 825)
MALPVTALLLPLALLLHAARPSDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 119 - protein, 251 aa, synthetic construct
  REGION 1..251: note = Description of sequence: Receptor; name: TPOR/MPLR(478-582).IL7Ra(316-459)
SDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 120 - protein, 251 aa, synthetic construct
  REGION 1..251: note = Description of sequence: Receptor; name: TPOR/MPLR(478-582; H499LS505N).IL7Ra(316-459)
SDPTRVETATETAWISLVTALLLVLGLNAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 121 - protein, 251 aa, synthetic construct
  REGION 1..251: note = Description of sequence: Receptor; name: TPOR/MPLR(478-582;S505N).IL7Ra(316-459)
SDPTRVETATETAWISLVTALHLVLGLNAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 122 - protein, 251 aa, synthetic construct
  REGION 1..251: note = Description of sequence: Receptor; name: TPOR/MPLR(478-582;H499LW515K).IL7Ra(316-459)
SDPTRVETATETAWISLVTALLLVLGLSAVLGLLLLRKQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 123 - protein, 251 aa, synthetic construct
  REGION 1..251: note = Description of sequence: Receptor; name: TPOR/MPLR(478-582;W515K).IL7Ra(316-459)
SDPTRVETATETAWISLVTALHLVLGLSAVLGLLLLRKQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 124 - protein, 251 aa, synthetic construct
  REGION 1..251: note = Description of sequence: Receptor; name: TPOR/MPLR(478-582;H499LS505NW515K).IL7Ra(316-459)
SDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 125 - protein, 251 aa, synthetic construct
  REGION 1..251: note = Description of sequence: Receptor; name: TPOR/MPLR(478-582;S505NW515K).IL7Ra(316-459)
SDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 126 - protein, 251 aa, synthetic construct
  REGION 1..251: note = Description of sequence: Receptor; name: TPOR/MPLR(478-582;H499LG509N).IL7Ra(316-459)
SDPTRVETATETAWISLVTALLLVLGLSAVLNLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 127 - protein, 251 aa, synthetic construct; identical to SEQ ID NO: 120
  REGION 1..251: note = Description of sequence: Receptor; name: TPOR/MPLR(478-582; H499LS505N).IL7Ra(316-459)

SEQ ID NO: 128 - protein, 326 aa, synthetic construct
  REGION 1..326: note = Description of sequence: Receptor; name: TPOR/MPLR(478-582;S505NW515K).IL2Rb(333-551)
SDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 129 - protein, 256 aa, synthetic construct
  REGION 1..256: note = Description of sequence: Receptor; name: TPOR/MPLR(478-582;S505NW515K).IL12Rb2(714-862)
SDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 130 - protein, 326 aa, synthetic construct
  REGION 1..326: note = Description of sequence: Receptor; name: TPOR/MPLR(478-582;H499LS505NW515K).IL2Rb(333-551)
SDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLLEVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 131 - protein, 256 aa, synthetic construct
  REGION 1..256: note = Description of sequence: Receptor; name: TPOR/MPLR(478-582;H499LS505NW515K).IL12Rb2(714-862)
SDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLLEVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 132 - protein, 158 aa, synthetic construct
  REGION 1..158: note = Description of sequence: Receptor; name: TPOR/MPLR(478-582;S505NW515K).IL12Rb2(775-825)
SDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLESDPKPENPACPWTVLPAGDLPTHDGYLPSNIDDLPSHEAPLADSLEELEPQ

SEQ ID NO: 133 - protein, 158 aa, synthetic construct
  REGION 1..158: note = Description of sequence: Receptor; name: TPOR/MPLR(478-582;H499L;S505NW515K).IL12Rb2(775-825)
SDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLLESDPKPENPACPWTVLPAGDLPTHDGYLPSNIDDLPSHEAPLADSLEELEPQ

SEQ ID NO: 134 - protein, 304 aa, synthetic construct
  REGION 1..304: note = Description of sequence: Receptor; name: TPOR/MPLR( 478-582;S505NW515K).IL7Ra(316-459).IL12Rb2(775-825)
SDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 135 - protein, 304 aa, synthetic construct
  REGION 1..304: note = Description of sequence: Receptor; name: TPOR/MPLR( 478-582;H499LS505NW515K).IL7Ra(316-459).IL12Rb2(775-825)
SDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 136 - protein, 326 aa, synthetic construct
  REGION 1..326: note = Description of sequence: Receptor; name: TPOR/MPLR(478-582;S505NW515K).IL2Rb(333-551; Y381S Y384SY387S)
SDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEVTQLLLQQDKVPEPASLSSNHSLTSCFTNQGYFFFHLPDALEIEACQVSFTSDPSSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 137 - protein, 326 aa, synthetic construct
  REGION 1..326: note = Description of sequence: Receptor; name: TPOR/MPLR( 478-582;H499LS505NW515K).IL2Rb(333-551;Y381SY384SY387S)
SDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLLEVTQLLLQQDKVPEPASLSSNHSLTSCFTNQGYFFFHLPDALEIEACQVSFTSDPSSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 138 - protein, 326 aa, synthetic construct
  REGION 1..326: note = Description of sequence: Receptor; name: TPOR/MPLR( 478-582;S505NW515K).IL2Rb(333-551;Y364SY381SY384SY387S)
SDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEVTQLLLQQDKVPEPASLSSNHSLTSCFTNQGSFFFHLPDALEIEACQVSFTSDPSSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 139 - protein, 326 aa, synthetic construct
  REGION 1..326: note = Description of sequence: Receptor; name: TPOR/MPLR( 478-582;H499LS505NW515K).IL2Rb(333-551;Y364SY381SY384SY387 S)
SDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLLEVTQLLLQQDKVPEPASLSSNHSLTSCFTNQGSFFFHLPDALEIEACQVSFTSDPSSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 140 - protein, 490 aa, synthetic construct
  REGION 1..490: note = Description of sequence: Domain; name: 2173 anti-EGFRvIII scFv
MALPVTALLLPLALLLHAARPEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 238 aa, synthetic construct
  REGION 1..238: note = Description of sequence: Domain; name: P5A2 anti-BCMA scFv
EVQLLESGGGLVQPGGSLRLSCAASGFTFSSYAMNWVRQAPGKGLEWVSAISDSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRASQSVSSSYLAWYQQKPGQAPRLLMYDASIRATGIPDRFSGSGSGTDFTLTISRLEPEDFAVYYCQQYGSWPLTFGQGTKVEIK

SEQ ID NO: 142 - protein, 519 aa, synthetic construct
  REGION 1..519: note = Description of sequence: Domain; name: P5A2 anti-BCMA CAR
MALPVTALLLPLALLLHAARPEVQLLESGGGLVQPGGSLRLSCAASGFTFSSYAMNWVRQAPGKGLEWVSAISDSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRASQSVSSSYLAWYQQKPGQAPRLLMYDASIRATGIPDRFSGSGSGTDFTLTISRLEPEDFAVYYCQQYGSWPLTFGQGTKVEIKGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 219 aa, synthetic construct
  REGION 1..219: note = Description of sequence: Cytotail sequence; name: IL2Rb(333-551 Y364SY381SY384SY387S)
VTQLLLQQDKVPEPASLSSNHSLTSCFTNQGSFFFHLPDALEIEACQVSFTSDPSSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 144 - protein, 13 aa, synthetic construct
  REGION 1..13: note = Description of sequence: Epitope tag, named v5
KPIPNPLLGLDST

(19) United States
(12) Patent Application Publication
(10) Pub. No.: US 2025/0051421 A1
(43) Pub. Date: Feb. 13, 2025

(54) CONSTITUTIVELY ACTIVE CHIMERIC CYTOKINE RECEPTORS

(71) Applicant: Allogene Therapeutics, Inc., South San Francisco, CA (US)

(72) Inventors: Regina Junhui LIN, San Mateo, CA (US); Thomas John VAN BLARCOM, Oakland, CA (US); Siler PANOWSKI, Berkeley, CA (US); Barbra Johnson SASU, San Francisco, CA (US)

(21) Appl. No.: 18/741,447

(22) Filed: Jun. 12, 2024

Related U.S. Application Data
(63) Continuation of application No. 16/804,917, filed on Feb. 28, 2020, now Pat. No. 12,043,655.
(60) Provisional application No. 62/980,823, filed on Feb. 24, 2020.
(60) Provisional application No. 62/812,911, filed on Mar. 1, 2019.

(51) Int. Cl. C07K 14/715; A61K 39/00; A61P 35/00; C07K 16/40; C12N 5/0783; C12N 7/00; C12N 15/86

(52) U.S. Cl. CPC C07K 14/715; A61K 39/4611; A61K 39/4631; A61K 39/4635; A61K 39/464404; A61K 39/464412; A61K 39/464417; A61P 35/00; C07K 16/40; C12N 5/0636; C12N 7/00; C12N 15/86; A61K 2239/31; A61K 2239/38; A61K 2239/48; C07K 2317/24; C07K 2317/73; C07K 2319/02; C07K 2319/03; C07K 2319/30; C07K 2319/33; C12N 2740/15043

(57) ABSTRACT

Provided herein are constitutively active chimeric cytokine receptors (CACCRs). When present on chimeric antigen receptor (CAR)-bearing immune cells, such CACCRs allow for increased immune cell activation, proliferation, persistence, and/or potency. Also provided are methods of making and using the CACCRs described herein.

### CROSS REFERENCE TO RELATED APPLICATIONS

The present application is a divisional of U.S. patent application Ser. No. 16/804,917, filed on Feb. 28, 2020, which claims the benefit of priority to U.S. Provisional Application No. 62/812,911, filed on Mar. 1, 2019; and U.S. Provisional Application No. 62/980,823, filed on Feb. 24, 2020, the contents of all of which are hereby incorporated by reference in their entireties.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Oct. 16, 2024, is entitled “AT-023_04US_ST26.xml”, and is 215,332 bytes in size.

### BACKGROUND

Adoptive transfer of immune cells (e.g. T-cells) genetically modified to recognize malignancy-associated antigens is showing promise as a new approach to treating cancer. For example, T-cells can be genetically modified to express chimeric antigen receptors (CARs), which are fusion proteins comprised of an antigen recognition moiety and T-cell activation domains.

T-cell proliferation, cytotoxic potency and persistence is driven by signal transduction pathways. Conventional CAR designs provide two signals—CD3zeta activation (Signal 1) and co-stimulation (Signal 2, e.g. via 4-1BB, OX40, and/or CD28 expression). In some contexts, a third signal (Signal 3), cytokine-induced cytokine receptor signaling (e.g. cytokine support for immune potentiation), may be desirable. Approaches to provide Signal 3 have however been met with significant limitations.

One approach to provide cytokine support includes combining CAR-T-cell therapy with systemic infusions of recombinant cytokines/cytokine mimetics, and engineering CAR-T-cells to secrete/express cytokines extracellularly. As cytokines have pleiotropic effects and can also impact the function of other cell types, the systemic administration or production of immune-potentiating cytokines by CAR-T-cells have at least two major drawbacks: (i) these approaches can cause systemic toxicity in humans, and (ii) in the context of allogeneic CAR-T-cell therapy, these approaches may cause bystander host immune-activation that could accelerate the rejection of allogeneic CAR-T-cells, thereby compromising therapeutic efficacy. Another approach to provide cytokine support was based on introducing a constitutively activated dimerized cytokine receptor, an IL-7Ra—this limits the nature (IL-7 signaling only) and magnitude of signaling output. Yet another approach to provide cytokine support involved incorporating Signal 3 directly into the CAR molecule (Nat Med. 2018 March; 24(3): 352-359.). A limitation of this approach is that the strength of Signal 3 depends on the strength of CAR activation. In the absence of target (and CAR activation), Signal 3 would not be transduced.

Needed are solutions to circumvent these drawbacks by targeting cytokine signals specifically to CAR-T cells in a tunable way, thus allowing for an improved safety profile and therapeutic efficacy. Provided herein are compositions and methods that address this need.

### SUMMARY

The present disclosure provides constitutively active chimeric cytokine receptors (CACCRs). When present on chimeric antigen receptor (CAR)-bearing immune cells (CAR-I cells, e.g. CAR-T-cells), such CACCRs allow for increased immune cell activation, proliferation, persistence, and/or potency. Also provided are methods of making and using the CACCRs described herein.

Accordingly, in one aspect, provided herein is a CACCR composed of two monomers, each monomer comprising: (a) a transmembrane domain; (b) a Janus Kinase (JAK)-binding domain; and (c) a recruiting domain, wherein the monomers are constitutively dimerized. In some embodiments, the CACCR does not comprise an extracellular domain ligand binding domain.

In some embodiments, the transmembrane domain and/or the JAK-binding domain is derived from the TPOR/MPLR receptor. In some embodiments, the transmembrane domain and/or the JAK binding domain is derived from amino acids 478-582 of the naturally occurring TPOR/MPLR receptor of SEQ ID NO: 6. In some embodiments, the TPOR/MPLR receptor comprises one or more of the amino acid substitutions selected from H499L, S505N, W515K, and G509N. In some embodiments, the TPOR/MPLR receptor comprises the H499L, S505N and W515K substitutions, or the S505N and W515K substitutions. In some embodiments, the recruiting domain is a STAT-recruiting domain. In some embodiments, the recruiting domain comprises the STAT-recruiting domain from IL7Ra, for example, IL7Ra(316-459). In some embodiments, the recruiting domain comprises the STAT-recruiting domain from IL2Rb, for example, IL2Rb(333-551), IL2Rb(393-433, 518-551), IL2Rb(339-379, 393-433, 518-551), IL2Rb(333-551, Y381S, Y384S, Y387S), IL2Rb(333-551, Y364S, Y381S, Y384S, Y387S). In some embodiments, the recruiting domain comprises the STAT-recruiting domain from IL12Rb1, for example, IL12Rb1(622-662). In some embodiments, the recruiting domain comprises the STAT-recruiting domain from IL12Rb2, for example, IL12Rb2(714-862) or IL12Rb2(775-825). In some embodiments, the recruiting domain comprises the STAT-recruiting domain from IL21R, for example, IL21R (322-538).

In a related aspect provided herein is a polynucleotide encoding any one of the CACCRs of the disclosure, and an expression vector comprising such polynucleotide. In some embodiments, the polynucleotide further encodes for a chimeric antigen receptor (CAR), wherein the CAR binds to BCMA, EGFRvIII, Flt-3, WT-1, CD20, CD23, CD30, CD38, CD70, CD33, CD133, LeY, NKG2D, CS1, CD44v6, RORI, CD19, Claudin-18.2 (Claudin-18A2, or Claudin 18 isoform 2), DLL3 (Delta-like protein 3, Drosophila Delta homolog 3, Delta3), Muc 17 (Mucin17, Muc3, Muc3), FAP alpha (Fibroblast Activation Protein alpha), Ly6G6D (Lymphocyte antigen 6 complex locus protein God, c6orf23, G6D, MEGT1, NG25), and/or RNF43 (E3 ubiquitin-protein ligase RNF43, RING finger protein 43).

In another aspect, provided herein is an engineered immune cells comprising at least one chimeric antigen receptor (CAR) and at least one CACCR of the disclosure. In some embodiments the immune cell is a T-cell. In some embodiments the immune cell is an allogeneic immune cell. In other embodiments, the immune cell is an autologous immune cell. The immune cell may be selected from the group consisting of: T-cell, dendritic cell, killer dendritic cell, mast cell, NK-cell, macrophage, monocyte, B-cell and an immune cell derived from a stem cell. In a related aspect, provided herein is a pharmaceutical composition comprising any of the engineered immune cells of the disclosure, and a kit comprising such a pharmaceutical composition.

In another aspect, provided herein is a method of treating a cancer in a subject, comprising administering to the subject a therapeutically effective amount of any of the engineered immune cells described herein.

### BRIEF DESCRIPTION OF THE DRAWINGS

FIG. 1 shows a schematic of an engineered CACCR of the disclosure.

FIG. 2 shows a schematic of a vector of the disclosure that can be used to co-express the CACCR and CAR of the disclosure. One or more cytotails may be joined in tandem to mimic signaling from one or more cytokines. A schematic diagram of the vector expressing the control BFP (blue fluorescent protein) CAR is also shown.

FIGS. 3A-3B show the identification of TpoR transmembrane (TM) mutants that constitutively activate cytokine receptor signaling.

FIGS. 4A-4C show results for the expansion of CAR-T-cells coexpressing a constitutively active chimeric cytokine receptor.

FIG. 5 shows differentiation and the memory T-cell subset distribution in the CAR-T-cell product, under different IL-2 conditions.

FIGS. 6A-6B show the extent of constitutive cytokine signaling mediated by each TpoR TM variant.

FIGS. 7A-7D show the cytotoxic activity of TpoR TM mutants, indicating that constitutive cytokine receptor signaling enhances CAR-T-cell potency.

FIGS. 8A-8B show the cytotoxic activity and durability of TpoR TM mutants.

FIG. 9 shows the enrichment of CAR-T-cells over time in a growth factor-independent assay.

FIGS. 10A-10B show the fold expansion of CAR-T-cells over time in a growth factor-independent assay.

FIG. 11 shows the memory T-cell subset distribution among CAR+ T-cells over time in a growth factor-independent assay.

FIG. 12 shows activation of STAT signaling pathways by CAR-T cells co-expressing indicated CACCR.

FIGS. 13A-13B and FIGS. 14A-14B show optimization of CACCR signaling strength shown in a reporter assay in HEK293 T cells expressing full-length or truncated cytotails.

FIG. 15 shows optimization of CACCR signaling strength shown in primary CAR-T cells co-expressing full-length or truncated cytotails.

FIGS. 16A-16C show that CACCR CAR-T cells bearing truncated IL2Rb cytotails more closely mimic IL-15, rather than IL-2, signaling.

FIGS. 17A-17D show combinatorial signal outputs of different cytotail fused in tandem.

FIGS. 18A-18B depict the effects of CACCRs on memory differentiation of CAR-T cells.

FIGS. 19A-19D depict the impact of CACCRs on CAR-T cell survival and memory differentiation under growth factor-independent conditions.

FIGS. 20A-20B depict cytotoxic activity of CAR-T cells co-expressing various CACCRs.

FIG. 21 shows that CACCRs improved the cytotoxic activity of CAR-T cells directed towards a liquid tumor target BCMA.

FIGS. 22A-22C show that CACCRs improved the in vivo anti-tumor activity and persistence of BCMA CAR-T cells against orthotopic multiple myeloma.

FIG. 23 shows that CACCRs improved the anti-tumor activity of CAR-T cells against established solid tumors.

### DETAILED DESCRIPTION

The present disclosure provides constitutively active chimeric cytokine receptors (CACCRs). The presence of a constitutively active, tunable chimeric cytokine receptor allows for the immune potentiation of Signal 3 to meet the need for immune potentiation. Accordingly, when present on chimeric antigen receptor (CAR)-bearing immune cells (CAR-I cells, e.g. CAR-T-cells), such CACCRs allow for increased immune cell activation, proliferation, persistence, and/or potency. Also provided herein are methods of making and using the CACCRs described herein.

The CACCRs of the disclosure are tunable, and have flexible cytokine signaling outputs for the enhancement of CAR-T cell activity, persistence, and the like. The components, methods of making and use are described in turn below.

### I. Constitutively Active Chimeric Cytokine Receptors (CACCRs)

The CACCRs of the disclosure are composed of two monomers, each monomer comprising: (a) transmembrane domain; (b) a JAK-binding domain; and (c) a recruiting domain, wherein the monomers are constitutively dimerized. In some embodiments, the CACCR of the disclosure does not comprise a extracellular ligand-binding domain.

In some embodiments, the monomers are identical, giving rise to a constitutively active homodimer. In such embodiments, the number of proteins that need to be expressed in a vector are reduced. In some embodiments, the monomers are not identical, giving rise a constitutively active heterodimer, which may be desirable under certain circumstances.

The monomers of the CACCRs of the disclosure are capable of spontaneously dimerizing, and can activate signaling in the absence of any exogenous stimulation or ligand (ligand-independent dimerization). The level of activity can be controlled by mutations introduced into the transmembrane domain of the CACCRs. A skilled artisan will appreciate that the monomers of the CACCRs are not dimerized 100% of the time, and may exist as a monomer.

### A. Transmembrane Domains

The CACCRs of the disclosure comprise transmembrane domains. The transmembrane domains of the disclosure contain sequences such that they allow for constitutive dimerization with the monomer pair, thus allowing constitutive JAK activation on the intracellular portion, and constitutive recruitment and phosphorylation of, for example, STAT on the cytoplasmic region of the receptor.

The transmembrane domains are on the N-terminus and are coupled to intracellular/cytoplasmic domains on the C-terminus. In some embodiments, the coupling is achieved optionally through a linker.

As used herein, the transmembrane domains are capable of insertion into the membrane of a cell in which it is expressed. In some embodiments, the transmembrane domains of the disclosure span a cellular membrane, and comprise an extracellular portion, and/or an intracellular portion.

In some embodiments, the transmembrane domains of the disclosure are engineered (synthetic) and do not resemble any naturally occurring transmembrane domain, e.g. they are non-naturally occurring.

In other embodiments, the transmembrane domains of the disclosure are derived from naturally occurring receptors.

In some embodiments, the transmembrane domains and/or JAK-activating domains of the disclosure are derived from, for example, one or more of the following receptors: erythropoietin receptor (EpoR), Interleukin 6 signal transducer (GP130 or IL6ST), prolactin receptor (PrlR), growth hormone receptor (GHR), granulocyte colony-stimulating factor receptor (GCSFR), and thrombopoietin receptor/myeloproliferative leukemia protein receptor (TPOR/MPLR). When derived from naturally occurring receptors, the entire receptor, or the entire transmembrane sequence of the receptor may not be necessary to effectuate constitutive activation and constitutive JAK binding/activation on the intracellular portion. Accordingly fragments of naturally occurring receptors may be utilized. Furthermore, certain mutations may be introduced into the transmembrane domains derived from naturally occurring receptors, to further tune the downstream signaling.

In some embodiments, the transmembrane domain and/or JAK-activating domain of the disclosure is derived from the naturally occurring EpoR receptor.

In some embodiments, the transmembrane domain and/or JAK-activating domain of the disclosure is derived from the naturally occurring GP130 receptor.

In some embodiments, the transmembrane domain and/or JAK-activating domain of the disclosure is derived from the naturally occurring PrIR receptor.

In some embodiments, the transmembrane domain and/or JAK-activating domain of the disclosure is derived from the naturally occurring GHR receptor.

In some embodiments, the transmembrane domain and/or JAK-activating domain of the disclosure is derived from the naturally occurring GCSF receptor.

In some embodiments, the transmembrane domain and/or JAK-activating domain of the disclosure is derived from the naturally occurring TPOR receptor.

Table 1a provides exemplary full-length sequences of naturally occurring receptors provided in the disclosure, from which the transmembrane proteins are derived. The sequences provided in Table 1a are reference sequences, in relation to which later mutations are expressed, for example in Tables 1b and 1c.

TABLE 1a
Exemplary Naturally Occurring Receptors

| | SEQ |
|---|---|
| Naturally Occurring Receptor Name | ID NO: |
| >AAI12154.1 Erythropoietin receptor [Homo sapiens] | 1 |
| MDHLGASLWPQVGSLCLLLAGAAWAPPPNLPDPKFESKAALLAARGPEELLCFTERLEDLV | |
| CFWEEAASA | |
| GVGPGNYSFSYQLEDEPWKLCRLHQAPTARGAVRFWCSLPTADTSSFVPLELRVTAASGAP | |
| RYHRVIHIN | |
| EVVLLDAPVGLVARLADESGHVVLRWLPPPETPMTSHIRYEVDVSAGNGAGSVQRVEILEG | |
| RTECVLSNL | |
| RGRTRYTFAVRARMAEPSFGGFWSAWSEPVSLLTPSDLDPLILTLSLILVVILVLLTVLALLS | |
| HRRALKQ | |
| KIWPGIPSPESEFEGLFTTHKGNFQLWLYQNDGCLWWSPCTPFTEDPPASLEVLSERCWGT | |
| MQAVEPGTD | |
| DEGPLLEPVGSEHAQDTYLVLDKWLLPRNPPSEDLPGPGGSVDIVAMDEGSEASSCSSALA | |
| SKPSPEGAS | |
| AASFEYTILDPSSQLLRPWTLCPELPPTPPHLKYLYLVVSDSGISTDYSSGDSQGAQGGLSDG | |
| PYSNPYE | |
| NSLIPAAEPLPPSYVACS | |
| >AAI17403.1 Interleukin 6 signal transducer (GP130, oncostatin M | 2 |
| receptor) [Homo sapiens] | |
| MLTLQTWLVQALFIFLTTESTGELLDPCGYISPESPVVQLHSNFTAVCVLKEKCMDYFHVN | |
| ANYIVWKTN | |
| HFTIPKEQYTIINRTASSVTFTDIASLNIQLTCNILTFGQLEQNVYGITIISGLPPEKPKNLSCIV | |
| NEGK | |
| KMRCEWDRGRETHLETNFTLKSEWATHKFADCKAKRDTPTSCTVDYSTVYFVNIEVWVE | |
| AENALGKVTSD | |
| HINFDPVYKVKPNPPHNLSVINSEELSSILKLTWTNPSIKSVIILKYNIQYRTKDASTWSQIPPE | |
| DTAST | |
| RSSFTVQDLKPFTEYVFRIRCMKEDGKGYWSDWSEEASGITYEDRPSKAPSFWYKIDPSHT | |
| QGYRTVQLV | |
| WKTLPPFEANGKILDYEVTLTRWKSHLQNYTVNATKLTVNLTNDRYVATLTVRNLVGKSD | |
| AAVLTIPACD | |
| FQATHPVMDLKAFPKDNMLWVEWTTPRESVKKYILEWCVLSDKAPCITDWQQEDGTVHR | |
| TYLRGNLAESK | |
| CYLITVTPVYADGPGSPESIKAYLKQAPPSKGPTVRTKKVGKNEAVLEWDQLPVDVQNGFI | |
| RNYTIFYRT | |
| IIGNETAVNVDSSHTEYTLSSLTSDTLYMVRMAAYTDEGGKDGPEFTFTTPKFAQGEIEAIV | |
| VPVCLAFL | |
| LTTLLGVLFCFNKRDLIKKHIWPNVPDPSKSHIAQWSPHTPPRHNFNSKDQMYSDGNFTDV | |
| SVVEIEAND | |
| KKPFPEDLKSLDLFKKEKINTEGHSSGIGGSSCMSSSRPSISSSDENESSQNTSSTVQYSTVVH | |
| SGYRHQ | |
| VPSVQVFSRSESTQPLLDSEERPEDLQLVDHVDGGDGILPRQQYFKQNCSQHESSPDISHFER | |
| SKQVSSV | |
| NEEDFVRLKQQISDHISQSCGSGQMKMFQEVSAADAFGPGTEGQVERFETVGMEAATDEG | |
| MPKSYLPQTV | |
| RQGGYMPQ | |
| >XP_011512371.1 prolactin receptor isoform X2 [Homo sapiens] | 3 |
| MKENVASATVFTLLLFLNTCLLNGQLPPGKPEIFKCRSPNKETFTCWWRPGTDGGLPTNYS | |
| LTYHREGET | |
| LMHECPDYITGGPNSCHFGKQYTSMWRTYIMMVNATNQMGSSFSDELYVDVTYIVQPDPP | |
| LELAVEVKQP | |
| EDRKPYLWIKWSPPTLIDLKTGWFTLLYEIRLKPEKAAEWEIHFAGQQTEFKILSLHPGQKY | |
| LVQVRCKP | |
| DHGYWSAWSPATFIQIPSDFTMNDTTVWISVAVLSAVICLIIVWAVALKGYSMVTCIFPPVP | |
| GPKIKGFD | |
| AHLLEKGKSEELLSALGCQDFPPTSDYEDLLVEYLEVDDSEDQHLMSVHSKEHPSQGMKPT | |
| YLDPDTDSG | |
| RGSCDSPSLLSEKCEEPQANPSTFYDPEVIEKPENPETTHTWDPQCISMEGKIPYFHAGGSKC | |
| STWPLPQ | |
| PSQHNPRSSYHNITDVCELAVGPAGAPATLLNEAGKDALKSSQTIKSREEGKATQQREVESF | |
| HSETDQDT | |
| PWLLPQEKTPFGSAKPLDYVEIHKVNKDGALSLLPKQRENSGKPKKPGTPENNKEYAKVSG | |
| VMDNNILVL | |
| VPDPHAKNVACFEESAKEAPPSLEQNQAEKALANFTATSSKCRLQLGGLDYLDPACFTHSF | |
| H | |
| >NP_000154.1 growth hormone receptor isoform 1 precursor | 4 |
| [Homo sapiens] | |
| MDLWQLLLTLALAGSSDAFSGSEATAAILSRAPWSLQSVNPGLKTNSSKEPKFTKCRSPERE | |
| TFSCHWTD | |
| EVHHGTKNLGPIQLFYTRRNTQEWTQEWKECPDYVSAGENSCYFNSSFTSIWIPYCIKLTSN | |
| GGTVDEKC | |
| FSVDEIVQPDPPIALNWTLLNVSLTGIHADIQVRWEAPRNADIQKGWMVLEYELQYKEVNE | |
| TKWKMMDPI | |
| LTTSVPVYSLKVDKEYEVRVRSKQRNSGNYGEFSEVLYVTLPQMSQFTCEEDFYFPWLLIII | |
| FGIFGLTV | |
| MLFVFLFSKQQRIKMLILPPVPVPKIKGIDPDLLKEGKLEEVNTILAIHDSYKPEFHSDDSWV | |
| EFIELDI | |
| DEPDEKTEESDTDRLLSSDHEKSHSNLGVKDGDSGRTSCCEPDILETDFNANDIHEGTSEVA | |
| QPQRLKGE | |
| ADLLCLDQKNQNNSPYHDACPATQQPSVIQAEKNKPQPLPTEGAESTHQAAHIQLSNPSSLS | |
| NIDFYAQV | |
| SDITPAGSVVLSPGQKNKAGMSQCDMHPEMVSLCQENFLMDNAYFCEADAKKCIPVAPHI | |
| KVESHIQPSL | |
| NQEDIYITTESLTTAAGRPGTGEHVPGSEMPVPDYTSIHIVQSPQGLILNATALPLPDKEFLSS | |
| CGYVST | |
| DQLNKIMP | |
| >XP_016855859.1 granulocyte colony-stimulating factor receptor | 5 |
| isoform X1 [Homo sapiens] | |
| MARLGNCSLTWAALIILLLPGSLEECGHISVSAPIVHLGDPITASCIIKQNCSHLDPEPQILWR | |
| LGAELQ | |
| PGGRQQRLSDGTQESIITLPHLNHTQAFLSCCLNWGNSLQILDQVELRAGYPPAIPHNLSCL | |
| MNLTTSSL | |
| ICQWEPGPETHLPTSFTLKSFKSRGNCQTQGDSILDCVPKDGQSHCCIPRKHLLLYQNMGIW | |
| VQAENALG | |
| TSMSPQLCLDPMDVVKLEPPMLRTMDPSPEAAPPQAGCLQLCWEPWQPGLHINQKCELRH | |
| KPQRGEASWA | |
| LVGPLPLEALQYELCGLLPATAYTLQIRCIRWPLPGHWSDWSPSLELRTTERAPTVRLDTW | |
| WRQRQLDPR | |
| TVQLFWKPVPLEEDSGRIQGYVVSWRPSGQAGAILPLCNTTELSCTFHLPSEAQEVALVAY | |
| NSAGTSRPT | |
| PVVFSESRGPALTRLHAMARDPHSLWVGWEPPNPWPQGYVIEWGLGPPSASNSNKTWRM | |
| EQNGRATGFLL | |
| KENIRPFQLYEIIVTPLYQDTMGPSQHVYAYSQEMAPSHAPELHLKHIGKTWAQLEWVPEP | |
| PELGKSPLT | |
| HYTIFWTNAQNQSFSAILNASSRGFVLHGLEPASLYHIHLMAASQAGATNSTVLTLMTLTPE | |
| GSELHIIL | |
| GLFGLLLLLTCLCGTAWLCCSPNRKNPLWPSVPDPAHSSLGSWVPTIMEELPGPRQGQWLG | |
| QTSEMSRAL | |
| TPHPCVQDAFQLPGLGTPPITKLTVLEEDEKKPVPWESHNSSETCGLPTLVQTYVLQGDPRA | |
| VSTQPQSQ | |
| SGTSDQVLYGQLLGSPTSPGPGHYLRCDSTQPLLAGLTPSPKSYENLWFQASPLGTLVTPAP | |
| SQEDDCVF | |
| GPLLNFPLLQGIRVHGMEALGSF | |
| >NP_005364.1 thrombopoietin receptor precursor [Homo sapiens] | 6 |
| MPSWALFMVTSCLLLAPQNLAQVSSQDVSLLASDSEPLKCFSRTFEDLTCFWDEEEAAPSG | |
| TYQLLYAYP | |
| REKPRACPLSSQSMPHFGTRYVCQFPDQEEVRLFFPLHLWVKNVFLNQTRTQRVLFVDSVG | |
| LPAPPSIIK | |
| AMGGSQPGELQISWEEPAPEISDFLRYELRYGPRDPKNSTGPTVIQLIATETCCPALQRPHSA | |
| SALDQSP | |
| CAQPTMPWQDGPKQTSPSREASALTAEGGSCLISGLQPGNSYWLQLRSEPDGISLGGSWGS | |
| WSLPVTVDL | |
| PGDAVALGLQCFTLDLKNVTCQWQQQDHASSQGFFYHSRARCCPRDRYPIWENCEEEEKT | |
| NPGLQTPQFS | |
| RCHFKSRNDSIIHILVEVTTAPGTVHSYLGSPFWIHQAVRLPTPNLHWREISSGHLELEWQHP | |
| SSWAAQE | |
| TCYQLRYTGEGHQDWKVLEPPLGARGGTLELRPRSRYRLQLRARLNGPTYQGPWSSWSDP | |
| TRVETATETA | |
| WISLVTALHLVLGLSAVLGLLLLRWQFPAHYRRLRHALWPSLPDLHRVLGQYLRDTAALS | |
| PPKATVSDTC | |
| EEVEPSLLEILPKSSERTPLPLCSSQAQMDYRRLQPSCLGTMPLSVCPPMAESGSCCTTHIAN | |
| HSYLPLS | |
| YWQQP | |

In some embodiments, the transmembrane domain of the disclosure is derived from a truncated version of the naturally occurring TPOR/MPLR (myeloproliferative leukemia protein) receptor show in Table 1a.

In some embodiments, the transmembrane domain of the CACCR comprises amino acids 478-582 of the TPOR receptor of Table 1a.

Table 1b provides exemplary transmembrane domain amino acid sequences of the disclosure, wherein the transmembrane domain is derived from the naturally occurring TPOR receptor.

TABLE 1b
Exemplary transmembrane domain amino
acid sequences

| Transmembrane | | SEQ |
|---|---|---|
| Domain | Amino acid sequence | ID |
| TPOR/MPLR(478-582) | SDPTRVETATETAWISLVTALHL | 7 |
| | VLGLSAVLGLLLLRWQFPAHYRR | |
| | LRHALWPSLPDLHRVLGQYLRDT | |
| | AALSPPKATVSDTCEEVEPSLLE | |
| | ILPKSSERTPLPL | |
| TPOR/MPLR(478-582; | SDPTRVETATETAWISLVTALLL | 8 |
| H499L, S505N) | VLGLNAVLGLLLLRWQFPAHYRR | |
| | LRHALWPSLPDLHRVLGQYLRDT | |
| | AALSPPKATVSDTCEEVEPSLLE | |
| | ILPKSSERTPLPL | |
| TPOR/MPLR(478- | SDPTRVETATETAWISLVTALHL | 9 |
| 582; S505N) | VLGLNAVLGLLLLRWQFPAHYRR | |
| | LRHALWPSLPDLHRVLGQYLRDT | |
| | AALSPPKATVSDTCEEVEPSLLE | |
| | ILPKSSERTPLPL | |
| TPOR/MPLR(478- | SDPTRVETATETAWISLVTALLL | 10 |
| 582; H499L, W515K) | VLGLSAVLGLLLLRKQFPAHYRR | |
| | LRHALWPSLPDLHRVLGQYLRDT | |
| | AALSPPKATVSDTCEEVEPSLLE | |
| | ILPKSSERTPLPL | |
| TPOR/MPLR(478- | SDPTRVETATETAWISLVTALHL | 11 |
| 582; W515K) | VLGLSAVLGLLLLRKQFPAHYRR | |
| | LRHALWPSLPDLHRVLGQYLRDT | |
| | AALSPPKATVSDTCEEVEPSLLE | |
| | ILPKSSERTPLPL | |
| TPOR/MPLR(478- | SDPTRVETATETAWISLVTALLL | 12 |
| 582; H499L, S505N, | VLGLNAVLGLLLLRKQFPAHYRR | |
| W515K) | LRHALWPSLPDLHRVLGQYLRDT | |
| | AALSPPKATVSDTCEEVEPSLLE | |
| | ILPKSSERTPLPL | |
| TPOR/MPLR(478- | SDPTRVETATETAWISLVTALHL | 13 |
| 582; S505N, W515K) | VLGLNAVLGLLLLRKQFPAHYRR | |
| | LRHALWPSLPDLHRVLGQYLRDT | |
| | AALSPPKATVSDTCEEVEPSLLE | |
| | ILPKSSERTPLPL | |
| TPOR/MPLR(478- | SDPTRVETATETAWISLVTALLL | 14 |
| 582; H499L, G509N) | VLGLSAVLNLLLLRWQFPAHYRR | |
| | LRHALWPSLPDLHRVLGQYLRDT | |
| | AALSPPKATVSDTCEEVEPSLLE | |
| | ILPKSSERTPLPL | |

In some embodiments, the transmembrane domain of the CACCR comprises amino acids 478-582 of the TPOR receptor, and an amino acid substitution at least at H499. In some embodiments, the transmembrane domain of the CACCR comprises amino acids 478-582 of the TPOR receptor, and the amino acid substitution H499L.

In some embodiments, the transmembrane domain of the CACCR comprises amino acids 478-582 of the TPOR receptor, and an amino acid substitution at least at S505. In some embodiments, the transmembrane domain of the CACCR comprises amino acids 478-582 of the TPOR receptor, and the amino acid substitution S505N.

In some embodiments, the transmembrane domain of the CACCR comprises amino acids 478-582 of the TPOR receptor, and an amino acid substitution at least at G509. In some embodiments, the transmembrane domain of the CACCR comprises amino acids 478-582 of the TPOR receptor, and the amino acid substitution G509N.

In some embodiments, the transmembrane domain of the CACCR comprises amino acids 478-582 of the TPOR receptor, and an amino acid substitution at least at W515. In some embodiments, the transmembrane domain of the CACCR comprises amino acids 478-582 of the TPOR receptor, and the amino acid substitution W515K.

In some embodiments, the transmembrane domain of the CACCR comprises amino acids 478-582 of the TPOR receptor, and an amino acid substitution at H499 and S505 (sequence provided in Table 1b).

In some embodiments, the transmembrane domain of the CACCR comprises amino acids 478-582 of the TPOR receptor, and an amino acid substitution at H499 and W515 (sequence provided in Table 1b).

In some embodiments, the transmembrane domain of the CACCR comprises amino acids 478-582 of the TPOR receptor, and an amino acid substitution at H499, S505, and W515 (sequence provided in Table 1b).

In some embodiments, the transmembrane domain of the CACCR comprises amino acids 478-582 of the TPOR receptor, and an amino acid substitution at S505, and W515 (sequence provided in Table 1b).

In some embodiments, the transmembrane domain of the CACCR comprises amino acids 478-582 of the TPOR receptor, and an amino acid substitution at H499 and G509 (sequence provided in Table 1b).

In some embodiments, the transmembrane domain of the CACCR comprises amino acids 478-582 of the TPOR receptor, and the amino acid substitutions H499L and S505N (sequence provided in Table 1b).

In some embodiments, the transmembrane domain of the CACCR comprises amino acids 478-582 of the TPOR receptor, and the amino acid substitutions H499L and W515K (sequence provided in Table 1b).

In some embodiments, the transmembrane domain of the CACCR comprises amino acids 478-582 of the TPOR receptor, and the amino acid substitutions H499L and G509N (sequence provided in Table 1b).

In some embodiments, the transmembrane domain of the CACCR comprises amino acids 478-582 of the TPOR receptor, and the amino acid substitutions S505N and W515K (sequence provided in Table 1b).

In some embodiments, the transmembrane domain of the CACCR comprises amino acids 478-582 of the TPOR receptor, and the amino acid substitutions H499L, S505N, and W515K (sequence provided in Table 1b).

In some embodiments, the transmembrane domain of the CACCR comprises amino acids 478-582 of the TPOR receptor, and an amino acid substitution at H499 and S505 (sequence provided in Table 1b).

The CACCRs of the disclosure are tunable, to achieve the level of Signal 3/immune potentiation required in a CAR-bearing immune cell (e.g. CAR-T-cell) and desired in a particular context or condition.

In some embodiments, a low level of STAT5 activation is desired in a CAR-bearing immune cell (e.g. CAR-T-cell). By way of example, in such embodiments, the transmembrane domain of the CACCR comprising amino acids 478-582 of the TPOR receptor, and the amino acid substitution S505N, W515K, or H499L/G509N may be introduced.

In some embodiments, an increased level of STAT5 activation is desired in a CAR-bearing immune cell (e.g. CAR-T-cell). By way of example, in such embodiments, the transmembrane domain of the CACCR comprising amino acids 478-582 of the TPOR receptor, and the amino acid substitutions H499L, S505N, and W515K may be introduced. By way of another example, in such embodiments, the transmembrane domain of the CACCR comprising amino acids 478-582 of the TPOR receptor, and the amino acid substitutions S505N and W515K may be introduced.

In some embodiments, increased differentiation into memory T cells is desired in a CAR-bearing immune cell (e.g. CAR-T-cell). By way of example, in such embodiments, the transmembrane domain of the CACCR comprising amino acids 478-582 of the TPOR receptor, and the amino acid substitutions W515K, or H499L/G509N may be introduced.

In some embodiments, increased differentiation into memory T cells is desired in a CAR-bearing immune cell (e.g. CAR-T-cell). By way of example, in such embodiments, the transmembrane domain of the CACCR comprising amino acids 478-582 of the TPOR receptor, and the amino acid substitutions S505N/W515K and H499L/S505N/W515K may be introduced.

Also substitutions to increase cytotoxic potency, durability of response, and increased persistence are provided herein, for example S505N/W515K and H499L/S505N/W515K substitutions.

TABLE 1c
Exemplary Transmembrane + JAK2 Binding Domain Sequences

| Transmembrane and | | SEQ |
|---|---|---|
| JAK2 binding domain | Amino acid sequence | ID NO: |
| GCSFR(614-710) | LTLMTLTPEGSELHIILGLFGLLLLLTCLCGTAWL | 15 |
| | CCSPNRKNPLWPSVPDPAHSSLGSWVPTIMEEDA | |
| | FQLPGLGTPPITKLTVLEEDEKKPVPWE | |
| GP130(609-700) | TTPKFAQGEIEAIVVPVCLAFLLTTLLGVLFCFNK | 16 |
| | RDLIKKHIWPNVPDPSKSHIAQWSPHTPPRHNFN | |
| | SKDQMYSDGNFTDVSVVEIEAND | |
| TPOR/MPLR(478-582) | SDPTRVETATETAWISLVTALHLVLGLSAVLGLL | 17 |
| | LLRWQFPAHYRRLRHALWPSLPDLHRVLGQYLR | |
| | DTAALSPPKATVSDTCEEVEPSLLEILPKSSERTPL | |
| | PL | |
| TPOR/MPLR(N−1) | SDPTRVETATETWISLVTALHLVLGLSAVLGLLL | 18 |
| | LRWQFPAHYRRLRHALWPSLPDLHRVLGQYLRD | |
| | TAALSPPKATVSDTCEEVEPSLLEILPKSSERTPLP | |
| | L | |
| TPOR/MPLR(N−2) | SDPTRVETATETISLVTALHLVLGLSAVLGLLLLR | 19 |
| | WQFPAHYRRLRHALWPSLPDLHRVLGQYLRDT | |
| | AALSPPKATVSDTCEEVEPSLLEILPKSSERTPLPL | |
| TPOR/MPLR(N−2 + 1) | SDPTRVETATETLISLVTALHLVLGLSAVLGLLLL | 20 |
| | RWQFPAHYRRLRHALWPSLPDLHRVLGQYLRDT | |
| | AALSPPKATVSDTCEEVEPSLLEILPKSSERTPLPL | |
| TPOR/MPLR(N−3) | SDPTRVETATETSLVTALHLVLGLSAVLGLLLLR | 21 |
| | WQFPAHYRRLRHALWPSLPDLHRVLGQYLRDT | |
| | AALSPPKATVSDTCEEVEPSLLEILPKSSERTPLPL | |
| TPOR/MPLR(N−4) | SDPTRVETATETLVTALHLVLGLSAVLGLLLLRW | 22 |
| | QFPAHYRRLRHALWPSLPDLHRVLGQYLRDTAA | |
| | LSPPKATVSDTCEEVEPSLLEILPKSSERTPLPL | |
| TPOR/MPLR(N−4 + 1) | SDPTRVETATETILVTALHLVLGLSAVLGLLLLR | 23 |
| | WQFPAHYRRLRHALWPSLPDLHRVLGQYLRDT | |
| | AALSPPKATVSDTCEEVEPSLLEILPKSSERTPLPL | |
| TPOR/MPLR(N−5) | SDPTRVETATETVTALHLVLGLSAVLGLLLLRW | 24 |
| | QFPAHYRRLRHALWPSLPDLHRVLGQYLRDTAA | |
| | LSPPKATVSDTCEEVEPSLLEILPKSSERTPLPL | |
| TPOR/MPLR(N−6) | SDPTRVETATETTALHLVLGLSAVLGLLLLRWQF | 25 |
| | PAHYRRLRHALWPSLPDLHRVLGQYLRDTAALS | |
| | PPKATVSDTCEEVEPSLLEILPKSSERTPLPL | |
| TPOR/MPLR(N−7) | SDPTRVETATETALHLVLGLSAVLGLLLLRWQFP | 26 |
| | AHYRRLRHALWPSLPDLHRVLGQYLRDTAALSP | |
| | PKATVSDTCEEVEPSLLEILPKSSERTPLPL | |
| TPOR/MPLR(N−8) | SDPTRVETATETLHLVLGLSAVLGLLLLRWQFPA | 27 |
| | HYRRLRHALWPSLPDLHRVLGQYLRDTAALSPP | |
| | KATVSDTCEEVEPSLLEILPKSSERTPLPL | |
| TPOR/MPLR(N−9) | SDPTRVETATETHLVLGLSAVLGLLLLRWQFPAH | 28 |
| | YRRLRHALWPSLPDLHRVLGQYLRDTAALSPPK | |
| | ATVSDTCEEVEPSLLEILPKSSERTPLPL | |
| TPOR/MPLR(N−10) | SDPTRVETATETLVLGLSAVLGLLLLRWQFPAHY | 29 |
| | RRLRHALWPSLPDLHRVLGQYLRDTAALSPPKA | |
| | TVSDTCEEVEPSLLEILPKSSERTPLPL | |
| TPOR/MPLR(N−11) | SDPTRVETATETVLGLSAVLGLLLLRWQFPAHYR | 30 |
| | RLRHALWPSLPDLHRVLGQYLRDTAALSPPKAT | |
| | VSDTCEEVEPSLLEILPKSSERTPLPL | |
| TPOR/MPLR(N−12) | SDPTRVETATETLGLSAVLGLLLLRWQFPAHYRR | 31 |
| | LRHALWPSLPDLHRVLGQYLRDTAALSPPKATV | |
| | SDTCEEVEPSLLEILPKSSERTPLPL | |
| TPOR/MPLR(N−13) | SDPTRVETATETGLSAVLGLLLLRWQFPAHYRRL | 32 |
| | RHALWPSLPDLHRVLGQYLRDTAALSPPKATVS | |
| | DTCEEVEPSLLEILPKSSERTPLPL | |
| TPOR/MPLR(N−14) | SDPTRVETATETLSAVLGLLLLRWQFPAHYRRLR | 33 |
| | HALWPSLPDLHRVLGQYLRDTAALSPPKATVSD | |
| | TCEEVEPSLLEILPKSSERTPLPL | |
| TPOR/MPLR(N−15) | SDPTRVETATETSAVLGLLLLRWQFPAHYRRLRH | 34 |
| | ALWPSLPDLHRVLGQYLRDTAALSPPKATVSDT | |
| | CEEVEPSLLEILPKSSERTPLPL | |
| TPOR/MPLR(N−16) | SDPTRVETATETAVLGLLLLRWQFPAHYRRLRH | 35 |
| | ALWPSLPDLHRVLGQYLRDTAALSPPKATVSDT | |
| | CEEVEPSLLEILPKSSERTPLPL | |
| TPOR/MPLR(N−17) | SDPTRVETATETVLGLLLLRWQFPAHYRRLRHA | 36 |
| | LWPSLPDLHRVLGQYLRDTAALSPPKATVSDTCE | |
| | EVEPSLLEILPKSSERTPLPL | |
| TPOR/MPLR(N−18) | SDPTRVETATETLGLLLLRWQFPAHYRRLRHAL | 37 |
| | WPSLPDLHRVLGQYLRDTAALSPPKATVSDTCEE | |
| | VEPSLLEILPKSSERTPLPL | |
| TPOR/MPLR(N + 1) | SDPTRVETATETAWLISLVTALHLVLGLSAVLGL | 38 |
| | LLLRWQFPAHYRRLRHALWPSLPDLHRVLGQYL | |
| | RDTAALSPPKATVSDTCEEVEPSLLEILPKSSERTP | |
| | LPL | |
| TPOR/MPLR(N + 2) | SDPTRVETATETAWVLISLVTALHLVLGLSAVLG | 39 |
| | LLLLRWQFPAHYRRLRHALWPSLPDLHRVLGQY | |
| | LRDTAALSPPKATVSDTCEEVEPSLLEILPKSSER | |
| | TPLPL | |
| TPOR/MPLR(N + 3) | SDPTRVETATETAWLVLISLVTALHLVLGLSAVL | 40 |
| | GLLLLRWQFPAHYRRLRHALWPSLPDLHRVLGQ | |
| | YLRDTAALSPPKATVSDTCEEVEPSLLEILPKSSE | |
| | RTPLPL | |
| TPOR/MPLR(N + 4) | SDPTRVETATETAWILVLISLVTALHLVLGLSAVL | 41 |
| | GLLLLRWQFPAHYRRLRHALWPSLPDLHRVLGQ | |
| | YLRDTAALSPPKATVSDTCEEVEPSLLEILPKSSE | |
| | RTPLPL | |
| TPOR/MPLR(N + 5) | SDPTRVETATETAWLILVLISLVTALHLVLGLSAV | 42 |
| | LGLLLLRWQFPAHYRRLRHALWPSLPDLHRVLG | |
| | QYLRDTAALSPPKATVSDTCEEVEPSLLEILPKSS | |
| | ERTPLPL | |
| TPOR/MPLR(N + 6) | SDPTRVETATETAWLLILVLISLVTALHLVLGLSA | 43 |
| | VLGLLLLRWQFPAHYRRLRHALWPSLPDLHRVL | |
| | GQYLRDTAALSPPKATVSDTCEEVEPSLLEILPKS | |
| | SERTPLPL | |
| TPOR/MPLR(N + 7) | SDPTRVETATETAWVLLILVLISLVTALHLVLGLS | 44 |
| | AVLGLLLLRWQFPAHYRRLRHALWPSLPDLHRV | |
| | LGQYLRDTAALSPPKATVSDTCEEVEPSLLEILPK | |
| | SSERTPLPL | |
| TPOR/MPLR(N + 8) | SDPTRVETATETAWLVLLILVLISLVTALHLVLGL | 45 |
| | SAVLGLLLLRWQFPAHYRRLRHALWPSLPDLHR | |
| | VLGQYLRDTAALSPPKATVSDTCEEVEPSLLEILP | |
| | KSSERTPLPL | |

### B. Janus Kinase (JAK)-Binding Domains

The CACCRs of the disclosure comprise intracellular JAK-binding domains. The JAK-binding domain is coupled to the C-terminus of the transmembrane domain, either directly, or via a linker. The JAK-binding domain is coupled to the transmembrane domain on the intracellular side of the chimeric cytokine receptor.

In some embodiments, the JAK-binding domain is a JAK-1-binding domain, a JAK-2 binding domain, a JAK-3 binding domain, or a TYK2 binding domain.

In some embodiments, the JAK-binding domains of the CACCRs of the disclosure are naturally occurring, and derived from a naturally occurring receptor.

In some embodiments, the JAK-binding domains of the CACCRs of the disclosure are synthetic.

Table 1b and Table 1c provide exemplary amino acid sequences for transmembrane and JAK2 binding domains of the disclosure. In some embodiments, the CACCR of the disclosure comprises a transmembrane and JAK2 binding domain comprising an amino acid sequence selected from the sequences in Tables 1b and 1c. In some embodiments, the CACCR of the disclosure comprises a transmembrane and JAK2 binding domain comprising an amino acid sequence that is at least 80%, 85%, 90%, 95%, 98%, 99%, or 100% identical to any one the sequences in Tables 1b and 1c.

### C. Recruiting Domains

The CACCRs of the disclosure comprise cytoplasmic recruiting domains. The recruiting domain can be a STAT-recruiting domain, an AP1-recruiting domain, a Myc/Max-recruiting domain; or an NFkB-recruiting domain. In some embodiments, the recruiting domain is a Signal Transducer and Activator of Transcription (STAT)-recruiting (STAT-activating) domains. e/g/from receptor tails (cytotails) or from cytokine receptor tails. These intracellular recruiting domains of the CACCRs of the disclosure allow for the propagation of Signal 3 in an immune cell comprising a CAR and a chimeric cytokine receptor (e.g. a CAR-T-cell with a chimeric cytokine receptor of the disclosure). Cytokine signaling propagated through the Stat-recruiting domain allows for the cytokine-based immune potentiation of the cell. In some embodiments, the immune-potentiation is homeostatic, e.g. signaling gives rise to increase in immune cells bearing the CAR. In some embodiments, the immune-potentiation is inflammatory, e.g. signaling gives rise to increase in the potency of the immune cells bearing the CAR. In some embodiments, the immune-potentiation prevents exhaustion, e.g. signaling maintains the long-term functionality of immune cells bearing the CAR.

In some embodiments, the recruiting domains of the disclosure are synthetic, and do not resemble any naturally occurring receptor fragment. In some embodiments, the immune-potentiation prevents exhaustion, e.g. signaling maintains the long-term functionality of immune cells bearing the CAR.

In some embodiments, the Stat-recruiting domains of the disclosure are synthetic, and do not resemble any naturally occurring receptor fragment.

In other embodiments, the Stat-recruiting domains of the disclosure are derived from cytoplasmic tails of naturally occurring receptors, e.g. derived from naturally occurring cytokine receptors. These cytoplasmic tails of naturally occurring receptors may be the regions downstream of the JAK-activating domains of the transmembrane domain of the receptor. The Stat-recruiting domains of the chimeric cytokine receptors comprise at least one STAT-recruiting domain from at least one receptor. In some embodiments, the Stat-recruiting domain comprises at least one STAT1-recruiting domain. In some embodiments, the Stat-recruiting domain comprises at least one STAT2-recruiting domain.

In some embodiments, the Stat-recruiting domain comprises at least one STAT3-recruiting domain. In some embodiments, the Stat-recruiting domain comprises at least one STAT4-recruiting domain. In some embodiments, the Stat-recruiting domain comprises at least one STAT5-recruiting domain. In some embodiments, the Stat-recruiting domain comprises at least one STAT6-recruiting domain. In some embodiments, the Stat-recruiting domain comprises at least one STAT7-recruiting domain.

In some embodiments, the naturally occurring receptor from which the Stat-recruiting domain is derived, is not a cytokine receptor.

In some embodiments, the naturally occurring receptor from which the Stat-recruiting domain is derived, is a cytokine receptor. Exemplary cytokine receptors through which T-cell-immune potentiating cytokines signal include, but are not limited to IL-2 receptor, IL-7 receptor, IL-15 receptor and IL-21 receptor. In alternative embodiments, the receptor from which the Stat-recruiting domain is derived, is not a cytokine receptor. By choosing the Stat-recruiting domain of the CACCR, the receptor can be redirected to signaling of choice.

In some embodiments, the CACCR of the disclosure comprises a recruiting domain connected to the C-terminus of the transmembrane/JAK2 binding domain, with or without a linker. In some embodiments, the linker comprises one or more amino acid residues.

Table 2a provides exemplary receptors from which recruiting domains of the CACCRs of the disclosure are derived. Table 2b provides exemplary amino acid sequences of recruiting domains of the disclosure. In some embodiments, the CACCR of the disclosure comprises a recruiting domain comprising the amino acid sequence selected from one or more of the receptor sequences in Table 2b. In some embodiments, the CACCR of the disclosure comprises a recruiting domain comprising an amino acid sequence that is at least 80%, 85%, 90%, 95%, 98%, 99%, or 100% identical to any one of the sequences in Table 2b.

TABLE 2a
Source of Recruiting Domains

BLNK
IL2RG
EGFR
EpoR
GHR
IFNAR1
IFNAR2
IFNAR1/2
IFNLR1
IL10R1
IL12Rb1
IL12Rb2
IL21R
IL2Rb
IL2small
IL7R
IL7Ra
IL9R
IL15R
IL21R

TABLE 2b
Recruiting Domain (Cytotail) Sequences

| | | SEQ |
|---|---|---|
| Cytotail sequences | Amino acid sequence | ID NO: |
| IL7R(316-459) | ARDEVEGFLQDTFPQQLEESEKQRLGGDVQSPN | 46 |
| | CPSEDVVITPESFGRDSSLTCLAGNVSACDAPILS | |
| | SSRSLDCRESGKNGPHVYQDLLLSLGTTNSTLPPP | |
| | FSLQSGILTLNPVAQGQPILTSLGSNQEEAYVTMS | |
| | SFYQNQ | |
| IL2Rb(333-551) | VTQLLLQQDKVPEPASLSSNHSLTSCFTNQGYFF | 47 |
| | FHLPDALEIEACQVYFTYDPYSEEDPDEGVAGAP | |
| | TGSSPQPLQPLSGEDDAYCTFPSRDDLLLFSPSLL | |
| | GGPSPPSTAPGGSGAGEERMPPSLQERVPRDWDP | |
| | QPLGPPTPGVPDLVDFQPPPELVLREAGEEVPDA | |
| | GPREGVSFPWSRPPGQGEFRALNARLPLNTDAYL | |
| | SLQELQGQDPTHLV | |
| IFNAR1(508-557) | ISTIATVEETNQTDEDHKKYSSQTSQDSGNYSNE | 48 |
| | DESESKTSEELQQDFV | |
| IFNAR2(310-515) | KKKVWDYNYDDESDSDTEAAPRTSGGGYTMHG | 49 |
| | LTVRPLGQASATSTESQLIDPESEEEPDLPEVDVE | |
| | LPTMPKDSPQQLELLSGPCERRKSPLQDPFPEEDY | |
| | SSTEGSGGRITFNVDLNSVFLRVLDDEDSDDLEA | |
| | PLMLSSHLEEMVDPEDPDNVQSNHLLASGEGTQ | |
| | PTFPSPSSEGLWSEDAPSDQSDTSESDVDLGDGYI | |
| | MR | |
| IFNAR1/2(IFNAR1 | ISTIATVEETNQTDEDHKKYSSQTSQDSGNYSNE | 50 |
| residues 508-557-IFNAR2 | DESESKTSEELQQDFVKKKVWDYNYDDESDSDT | |
| residues 310-515) | EAAPRTSGGGYTMHGLTVRPLGQASATSTESQLI | |
| | DPESEEEPDLPEVDVELPTMPKDSPQQLELLSGPC | |
| | ERRKSPLQDPFPEEDYSSTEGSGGRITFNVDLNSV | |
| | FLRVLDDEDSDDLEAPLMLSSHLEEMVDPEDPD | |
| | NVQSNHLLASGEGTQPTFPSPSSEGLWSEDAPSD | |
| | QSDTSESDVDLGDGYIMR | |
| IFNLR1(300-520) | RGVRPTPRVRAPATQQTRWKKDLAEDEEEEDEE | 51 |
| | DTEDGVSFQPYIEPPSFLGQEHQAPGHSEAGGVD | |
| | SGRPRAPLVPSEGSSAWDSSDRSWASTVDSSWD | |
| | RAGSSGYLAEKGPGQGPGGDGHQESLPPPEFSKD | |
| | SGFLEELPEDNLSSWATWGTLPPEPNLVPGGPPV | |
| | SLQTLTFCWESSPEEEEEARESEIEDSDAGSWGAE | |
| | STQRTEDRGRTLGHYMAR | |
| IL2RG(335-369) | IPPKGGALGEGPGASPCNQHSPYWAPPCYTLKPE | 52 |
| | T | |
| IL9R(356-521) | TALLTCGPARPWKSVALEEEQEGPGTRLPGNLSS | 53 |
| | EDVLPAGCTEWRVQTLAYLPQEDWAPTSLTRPA | |
| | PPDSEGSRSSSSSSSSNNNNYCALGCYGGWHLSA | |
| | LPGNTQSSGPIPALACGLSCDHQGLETQQGVAW | |
| | VLAGHCQRPGLHEDLQGMLLPSVLSKARSWTF | |
| IL21R(322-538) | PRSPAKRLQLTELQEPAELVESDGVPKPSFWPTA | 54 |
| | QNSGGSAYSEERDRPYGLVSIDTVTVLDAEGPCT | |
| | WPCSCEDDGYPALDLDAGLEPSPGLEDPLLDAG | |
| | TTVLSCGCVSAGSPGLGGPLGSLLDRLKPPLADG | |
| | EDWAGGLPWGGRSPGGVSESEAGSPLAGLDMD | |
| | TFDSGFVGSDCSSPVECDFTSPGDEGPPRSYLRQ | |
| | WVVIPPPLSSPGPQAS | |
| GHR(353-638) | PDEKTEESDTDRLLSSDHEKSHSNLGVKDGDSGR | 55 |
| | TSCCEPDILETDFNANDIHEGTSEVAQPQRLKGE | |
| | ADLLCLDQKNQNNSPYHDACPATQQPSVIQAEK | |
| | NKPQPLPTEGAESTHQAAHIQLSNPSSLSNIDFYA | |
| | QVSDITPAGSVVLSPGQKNKAGMSQCDMHPEM | |
| | VSLCQENFLMDNAYFCEADAKKCIPVAPHIKVES | |
| | HIQPSLNQEDIYITTESLTTAAGRPGTGEHVPGSE | |
| | MPVPDYTSIHIVQSPQGLILNATALPLPDKEFLSS | |
| | CGYVSTDQLNKIMP | |
| EpoR(339-508) | WGTMQAVEPGTDDEGPLLEPVGSEHAQDTYLVL | 56 |
| | DKWLLPRNPPSEDLPGPGGSVDIVAMDEGSEASS | |
| | CSSALASKPSPEGASAASFEYTILDPSSQLLRPWT | |
| | LCPELPPTPPHLKYLYLVVSDSGISTDYSSGDSQG | |
| | AQGGLSDGPYSNPYENSLIPAAEPLPPSYVACS | |
| murine IL2Rb(337-539) | AVQLLLLQKDSAPLPSPSGHSQASCFTNQGYFFF | 57 |
| | HLPNALEIESCQVYFTYDPCVEEEVEEDGSRLPE | |
| | GSPHPPLLPLAGEQDDYCAFPPRDDLLLFSPSLST | |
| | PNTAYGGSRAPEERSPLSLHEGLPSLASRDLMGL | |
| | QRPLERMPEGDGEGLSANSSGEQASVPEGNLHG | |
| | QDQDRGQGPILTLNTDAYLSLQELQAQDSVHLI | |
| murine IL7Ra(316-459) | ARDEVESFLPNDLPAQPEELETQGHRAAVHSAN | 58 |
| | RSPETSVSPPETVRRESPLRCLARNLSTCNAPPLL | |
| | SSRSPDYRDGDRNRPPVYQDLLPNSGNTNVPVPV | |
| | PQPLPFQSGILIPVSQRQPISTSSVLNQEEAYVTMS | |
| | SFYQNK | |
| EGFR(955-1186) | VIQGDERMHLPSPTDSNFYRALMDEEDMDDVVD | 59 |
| | ADEYLIPQQGFFSSPSTSRTPLLSSLSATSNNSTVA | |
| | CIDRNGLQSCPIKEDSFLQRYSSDPTGALTEDSID | |
| | DTFLPVPEYINQSVPKRPAGSVQNPVYHNQPLNP | |
| | APSRDPHYQDPHSTAVGNPEYLNTVQPTCVNSTF | |
| | DSPAHWAQKGSHQISLDNPDYQQDFFPKEAKPN | |
| | GIFKGSTAENAEYLRVAPQSSEFIGA | |
| EGFR(955- | VIQGDERMHLPSPTDSNFFRALMDEEDMDDVVD | 60 |
| 1186; Y974F, d1045-1057) | ADEYLIPQQGFFSSPSTSRTPLLSSLSATSNNSTVA | |
| | CIDRNGLQSCPIKEDSFLQRIDDTFLPVPEYINQSV | |
| | PKRPAGSVQNPVYHNQPLNPAPSRDPHYQDPHS | |
| | TAVGNPEYLNTVQPTCVNSTFDSPAHWAQKGSH | |
| | QISLDNPDYQQDFFPKEAKPNGIFKGSTAENAEY | |
| | LRVAPQSSEFIGA | |
| EGFR(955-1009; Y974F) | VIQGDERMHLPSPTDSNFFRALMDEEDMDDVVD | 61 |
| | ADEYLIPQQGFFSSPSTSRTP | |
| EGFR(1019-1085) | NNSTVACIDRNGLQSCPIKEDSFLQRIDDTFLPVP | 62 |
| | EYINQSVPKRPAGSVQNPV | |
| EGFR(1037-1103; | KEDSFLQRIDDTFLPVPEFINQSVPKRPAGSVQNP | 63 |
| Y1068/1101F, d1045-1057) | VYHNQPLNPAPSRDPHFQD | |
| EGFR(1066- | VPEFINQSVPKRPAGSVQNPVFHNQPLNPAPSRD | 64 |
| 1118; Y1068/1086F) | PHYQDPHSTAVGNPEYLNTV | |
| EGFR(1122-1165) | PEYLNTVQPTCVNSTFDSPAHWAQKGSHQISLDN | 65 |
| | PDYQQDFFPKEAKPNGIFKG | |
| EGFR(1133- | WAQKGSHQISLDNPDFQQDFFPKEAKPNGIFKGS | 66 |
| 1186; Y1148F) | TAENAEYLRVAPQSSEFIGA | |
| IL12Rb2(775-825) | SDPKPENPACPWTVLPAGDLPTHDGYLPSNIDDL | 67 |
| | PSHEAPLADSLEELEPQ | |
| IL7Ra(376-416) | ACDAPILSSSRSLDCRESGKNGPHVYQDLLLSLG | 68 |
| | TTNSTLP | |
| IL7Ra(424-459) | GILTLNPVAQGQPILTSLGSNQEEAYVTMSSFYQ | 69 |
| | NQ | |
| IL7Ra(376-416, 424-459) | ACDAPILSSSRSLDCRESGKNGPHVYQDLLLSLG | 70 |
| | TTNSTLPQGQPILTSLGSNQEEAYVTMSSFYQNQ | |
| IL7Ra(424-459; Y456F) | GILTLNPVAQGQPILTSLGSNQEEAYVTMSSFFQN | 71 |
| | Q | |
| IL7R(376-416, 424- | ACDAPILSSSRSLDCRESGKNGPHVYQDLLLSLG | 72 |
| 459, Y456F) | TTNSTLPQGQPILTSLGSNQEEAYVTMSSFFQNQ | |
| IL2Rbsmall(393-433) | DEGVAGAPTGSSPQPLQPLSGEDDAYCTFPSRDD | 73 |
| | LLLFSPSGQGEFRALNARLPLNTDAYLSLQELQG | |
| | QDPTHLV | |
| IL2Rbsmall(518-551) | GQGEFRALNARLPLNTDAYLSLQELQGQDPTHL | 74 |
| | V | |
| IL2Rbsmall(339-379, 393- | QQDKVPEPASLSSNHSLTSCFTNQGYFFFHLPDA | 75 |
| 433) | LEIEACQDEGVAGAPTGSSPQPLQPLSGEDDAYC | |
| | TFPSRDDLLLFSPS | |
| IL2Rbsmall(339-379, 518- | QQDKVPEPASLSSNHSLTSCFTNQGYFFFHLPDA | 76 |
| 551) | LEIEACQ | |
| | GQGEFRALNARLPLNTDAYLSLQELQGQDPTHL | |
| | V | |
| IL2Rbsmall(393-433, 518- | DEGVAGAPTGSSPQPLQPLSGEDDAYCTFPSRDD | 77 |
| 551) | LLLFSPSGQGEFRALNARLPLNTDAYLSLQELQG | |
| | QDPTHLV | |
| IL2Rbsmall(339-379, 393- | QQDKVPEPASLSSNHSLTSCFTNQGYFFFHLPDA | 78 |
| 433, 518-551) | LEIEACQDEGVAGAPTGSSPQPLQPLSGEDDAYC | |
| | TFPSRDDLLLFSPSGQGEFRALNARLPLNTDAYLS | |
| | LQELQGQDPTHLV | |
| IFNAR2small(310-352) | KKKVWDYNYDDESDSDTEAAPRTSGGGYTMHG | 79 |
| | LTVRPLGQASA | |
| IFNAR2small(486-515) | EGLWSEDAPSDQSDTSESDVDLGDGYIMR | 80 |
| IFNAR2small(310-352, | KKKVWDYNYDDESDSDTEAAPRTSGGGYTMHG | 81 |
| 486-515) | LTVRPLGQASA | |
| | EGLWSEDAPSDQSDTSESDVDLGDGYIMR | |
| BLNK(53-208) | ASESPADEEEQWSDDFDSDYENPDEHSDSEMYV | 82 |
| | MPAEENADDSYEPPPVEQETRPVHPALPFARGEY | |
| | IDNRSSQRHSPPFSKTLPSKPSWPSEKARLTSTLP | |
| | ALTALQKPQVPPKPKGLLEDEADYVVPVEDNDE | |
| | NYIHPTESSSPPPEKAPMVNR | |
| BLNK(53-208; Y72F) | ASESPADEEEQWSDDFDSDFENPDEHSDSEMYV | 83 |
| | MPAEENADDSYEPPPVEQETRPVHPALPFARGEY | |
| | IDNRSSQRHSPPFSKTLPSKPSWPSEKARLTSTLP | |
| | ALTALQKPQVPPKPKGLLEDEADYVVPVEDNDE | |
| | NYIHPTESSSPPPEKAPMVNR | |
| BLNK(53- | ASESPADEEEQWSDDFDSDFENPDEHSDSEMYV | 84 |
| 208; Y72F, Y96F) | MPAEENADDSFEPPPVEQETRPVHPALPFARGEY | |
| | IDNRSSQRHSPPFSKTLPSKPSWPSEKARLTSTLP | |
| | ALTALQKPQVPPKPKGLLEDEADYVVPVEDNDE | |
| | NYIHPTESSSPPPEKAPMVNR | |
| EpoR(339-508) | WGTMQAVEPGTDDEGPLLEPVGSEHAQDTYLVL | 85 |
| | DKWLLPRNPPSEDLPGPGGSVDIVAMDEGSEASS | |
| | CSSALASKPSPEGASAASFEYTILDPSSQLLRPWT | |
| | LCPELPPTPPHLKYLYLVVSDSGISTDYSSGDSQG | |
| | AQGGLSDGPYSNPYENSLIPAAEPLPPSYVACS | |
| IL12Rb2(714-862) | VTPVFRHPPCSNWPQREKGIQGHQASEKDMMHS | 86 |
| | ASSPPPPRALQAESRQLVDLYKVLESRGSDPKPE | |
| | NPACPWTVLPAGDLPTHDGYLPSNIDDLPSHEAP | |
| | LADSLEELEPQHISLSVFPSSSLHPLTFSCGDKLTL | |
| | DQLKMRCDSLML | |
| IL12Rb1(622-662) | WDKGERTEPLEKTELPEGAPELALDTELSLEDGD | 87 |
| | RCKAKM | |
| IL10R1(304-578) | VSPELKNLDLHGSTDSGFGSTKPSLQTEEPQFLLP | 88 |
| | DPHPQADRTLGNREPPVLGDSCSSGSSNSTDSGIC | |
| | LQEPSLSPSTGPTWEQQVGSNSRGQDDSGIDLVQ | |
| | NSEGRAGDTQGGSALGHHSPPEPEVPGEEDPAA | |
| | VAFQGYLRQTRCAEEKATKTGCLEEESPLTDGL | |
| | GPKFGRCLVDEAGLEIPPALAKGYLKQDPLEMTL | |
| | ASSGAPTGQWNQPTEEWSLLALSSCSDLGISDWS | |
| | FAHDLAPLGCVAAPGGLLGSFNSDLVTLPLISSLQ | |
| | SSE | |
| IL2Rb(333-551, | VTQLLLQQDKVPEPASLSSNHSLTSCFTNQGYFF | 106 |
| Y381S, Y384S, Y387S) | FHLPDALEIEACQVSFTSDPSSEEDPDEGVAGAPT | |
| | GSSPQPLQPLSGEDDAYCTFPSRDDLLLFSPSLLG | |
| | GPSPPSTAPGGSGAGEERMPPSLQERVPRDWDPQ | |
| | PLGPPTPGVPDLVDFQPPPELVLREAGEEVPDAG | |
| | PREGVSFPWSRPPGQGEFRALNARLPLNTDAYLS | |
| | LQELQGQDPTHLV | |
| IL2Rb(333-551, | VTQLLLQQDKVPEPASLSSNHSLTSCFTNQGSFFF | 143 |
| Y364S, Y381S, Y384S, | HLPDALEIEACQVSFTSDPSSEEDPDEGVAGAPTG | |
| Y387S) | SSPQPLQPLSGEDDAYCTFPSRDDLLLFSPSLLGG | |
| | PSPPSTAPGGSGAGEERMPPSLQERVPRDWDPQP | |
| | LGPPTPGVPDLVDFQPPPELVLREAGEEVPDAGP | |
| | REGVSFPWSRPPGQGEFRALNARLPLNTDAYLSL | |
| | QELQGQDPTHLV | |

In some embodiments, the Stat-recruiting domain of a CACCR of the disclosure comprises a STAT-recruiting domain from one receptor.

In order to generate multiple outputs, two or more STAT-recruiting domains May be joined in tandem to mimic signaling from one or more cytokines.

In some embodiments, two or more STAT-recruiting domains may be joined in tandem with or without a linker. In some embodiments, the linker comprises one or more amino acid residues.

In some embodiments, the STAT-recruiting domain comprises portions of more than one receptor, e.g. comprising more than one STAT-recruiting domain. In such embodiments, a tandem cytokine signaling domain is provided, allowing for enhanced signaling. Accordingly, in some embodiments, the STAT-recruiting domain of a monomer of the CACCR of the disclosure comprises the STAT-recruiting domains from more than one receptor, e.g. comprises the STAT-recruiting domains from two, three, four, five, or even six receptors. For example, in some embodiments, STAT-recruiting domains can be linked in tandem to stimulate multiple pathways (e.g., the IL7R (316-459)-IL12Rb2(775-825) fragment fusion for pro-persistence STAT5 and pro-inflammatory STAT4; IL7R (316-459)-IL2Rbsmall(393-433,518-551) for pro-persistence; IL7R (316-459)-EGFR (1122-1165) for pro-persistence and anti-exhaustion; IL2Rbsmall(393-433,518-551)-EGFR (1122-1165) for pro-persistence and anti-exhaustion).

When generating multiple outputs, the proximity of individual STAT-recruiting domains to the cell membrane can influence the strength of their respective signaling outputs. Table 2c shows examples of CACCRs with the dual outputs, where each output can be placed either proximal or distal to the cell membrane. In some embodiments, the CACCRs of the disclosure comprise a recruiting domain with dual outputs selected from Table 2c.

TABLE 2c
Examples of CACCRs with dual outputs

| | Membrane | Membrane |
|---|---|---|
| Dual output STAT-recruiting domain | proximal | distal |
| IL2Rbsmall (393-433, 518-551)/IL21R (322-538) | IL2Rbsmall | IL21R (322- |
| | (393- | 538) |
| | 433, 518- | |
| | 551) | |
| IL2Rb (333-551)/IL21R (322-538) | IL2Rb (333- | IL21R (322- |
| | 551) | 538) |
| IL21R (322-538)/IL2Rbsmall (393-433, 518-551) | IL21R (322- | IL2Rbsmall |
| | 538) | (393- |
| | | 433, 518- |
| | | 551) |
| IL21R (322-538)/IL2Rb (333-551) | IL21R (322- | IL2Rb (333- |
| | 538) | 551) |
| IL2Rbsmall (339-379, 393-433, 518-551)/IL21R | IL2Rbsmall | IL21R (322- |
| (322-538) | (339- | 538) |
| | 379, 393- | |
| | 433, 518- | |
| | 551) | |
| IL21R (322-538)/IL2Rbsmall (339-379, 393-433, | IL21R (322- | IL2Rbsmall |
| 518-551) | 538) | (339- |
| | | 379, 393- |
| | | 433, 518- |
| | | 551) |
| IL2Rb (333-551)/IL12Rb1 (622-662) | IL2Rb (333- | IL12Rb1 (622- |
| | 551) | 662) |
| IL2Rbsmall (393-433, 518-551)/IL12Rb1 (622-662) | IL2Rbsmall | IL12Rb1 (622- |
| | (393- | 662) |
| | 433, 518- | |
| | 551) | |
| IL2Rbsmall (339-379, 393-433, 518-551)/IL12Rb1 | IL2Rbsmall | IL12Rb1 (622- |
| (622-662) | (339- | 662) |
| | 379, 393- | |
| | 433, 518- | |
| | 551) | |
| IL12Rb1 (622-662)/IL2Rb (333-551) | IL12Rb1 (622- | IL2Rb (333- |
| | 662) | 551) |
| IL12Rb1 (622-662)/IL2Rbsmall (393-433, 518-551) | IL12Rb1 (622- | IL2Rbsmall |
| | 662) | (393- |
| | | 433, 518- |
| | | 551) |
| IL12Rb1 (622-662)/IL2Rbsmall (339-379, 393-433, | IL12Rb1 (622- | IL2Rbsmall |
| 518-551) | 662) | (339- |
| | | 379, 393- |
| | | 433, 518- |
| | | 551) |
| IL2Rb (333-551)/IL12Rb2 (714-862) | IL2Rb (333- | IL12Rb2 (714- |
| | 551) | 862) |
| IL2Rbsmall (393-433, 518-551)/IL12Rb2 (714-862) | IL2Rbsmall | IL12Rb2 (714- |
| | (393- | 862) |
| | 433, 518- | |
| | 551) | |
| IL2Rbsmall (339-379, 393-433, 518-551)/IL12Rb2 | IL2Rbsmall | IL12Rb2 (714- |
| (714-862) | (339- | 862) |
| | 379, 393- | |
| | 433, 518- | |
| | 551) | |
| IL2Rb (333-551)/IL12Rb2 (775-825) | IL2Rb (333- | IL12Rb2 (775- |
| | 551) | 825) |
| IL2Rbsmall (393-433, 518-551)/IL12Rb2 (775-825) | IL2Rbsmall | IL12Rb2 (775- |
| | (393- | 825) |
| | 433, 518- | |
| | 551) | |
| IL2Rbsmall (339-379, 393-433, 518-551)/IL12Rb2 | IL2Rbsmall | IL12Rb2 (775- |
| (775-825) | (339- | 825) |
| | 379, 393- | |
| | 433, 518- | |
| | 551) | |
| IL12Rb2 (714-862)/IL2Rb (333-551) | IL12Rb2 (714- | IL2Rb (333- |
| | 862) | 551) |
| IL12Rb2 (714-862)/IL2Rbsmall (393-433, 518-551) | IL12Rb2 (714- | IL2Rbsmall |
| | 862) | (393- |
| | | 433, 518- |
| | | 551) |
| IL12Rb2 (714-862)/IL2Rbsmall (339-379, 393-433, 518- | IL12Rb2 (714- | IL2Rbsmall |
| 551) | 862) | (339- |
| | | 379, 393- |
| | | 433, 518- |
| | | 551) |
| IL12Rb2 (775-825)/IL2Rb (333-551) | IL12Rb2 (775- | IL2Rb (333- |
| | 825) | 551) |
| IL12Rb2 (775-825)/IL2Rbsmall (393-433, 518-551) | IL12Rb2 (775- | IL2Rbsmall |
| | 825) | (393- |
| | | 433, 518- |
| | | 551) |
| IL12Rb2 (775-825)/IL2Rbsmall (339-379, 393-433, 518- | IL12Rb2 (775- | IL2Rbsmall |
| 551) | 825) | (339- |
| | | 379, 393- |
| | | 433, 518- |
| | | 551) |
| IL7Ra (316-459)/IL21R (322-538) | IL7Ra (316- | IL21R (322- |
| | 459) | 538) |
| IL7Ra (376-416, 424-459, Y456F)/IL21R (322-538) | IL7Ra (376- | IL21R (322- |
| | 416, 424- | 538) |
| | 459, | |
| | Y456F) | |
| IL21R (322-538)/IL7Ra (316-459) | IL21R (322- | IL7Ra (316- |
| | 538) | 459) |
| IL21R (322-538)/IL7Ra (376-416, 424-459, Y456F) | IL21R (322- | IL7Ra (376- |
| | 538) | 416, 424- |
| | | 459, |
| | | Y456F) |
| IL7Ra (316-459)/IL12Rb1 (622-662) | IL7Ra (316- | IL12Rb1 (622- |
| | 459) | 662) |
| IL7Ra (376-416, 424-459, Y456F)/IL12Rb1 (622-662) | IL7Ra (376- | IL12Rb1 (622- |
| | 416, 424- | 662) |
| | 459, | |
| | Y456F) | |
| IL7Ra (316-459)/IL12Rb2 (714-862) | IL7Ra (316- | IL12Rb2 (714- |
| | 459) | 862) |
| IL7Ra (376-416, 424-459, Y456F)/IL12Rb2 (714-862) | IL7Ra (376- | IL12Rb2 (714- |
| | 416, 424- | 862) |
| | 459, | |
| | Y456F) | |
| IL7Ra (316-459)/IL12Rb2 (775-825) | IL7Ra (316- | IL12Rb2 (775- |
| | 459) | 825) |
| IL7Ra (376-416, 424-459, Y456F)/IL12Rb2 (775-825) | IL7Ra (376- | IL12Rb2 (775- |
| | 416, 424- | 825) |
| | 459, | |
| | Y456F) | |
| IL12Rb1 (622-662)/IL7Ra (316-459) | IL12Rb1 (622- | IL7Ra (316- |
| | 662) | 459) |
| IL12Rb1 (622-662)/IL7Ra (376-416, 424-459, Y456F) | IL12Rb1 (622- | IL7Ra (376- |
| | 662) | 416, 424- |
| | | 459, |
| | | Y456F) |
| IL12Rb2 (714-862)/IL7Ra (316-459) | IL12Rb2 (714- | IL7Ra (316- |
| | 862) | 459) |
| IL12Rb2 (714-862)/IL7Ra (376-416, 424-459, Y456F) | IL12Rb2 (714- | IL7Ra (376- |
| | 862) | 416, 424- |
| | | 459, |
| | | Y456F) |
| IL12Rb2 (775-825)/IL7Ra (316-459) | IL12Rb2 (775- | IL7Ra (316- |
| | 825) | 459) |
| IL12Rb2 (775-825)/IL7Ra (376-416, 424-459, Y456F) | IL12Rb2 (775- | IL7Ra (376- |
| | 825) | 416, 424- |
| | | 459, |
| | | Y456F) |
| IL7Ra (316-459)/IL2Rb (333-551) | IL7Ra (316- | IL2Rb (333- |
| | 459) | 551) |
| IL7Ra (376-416, 424-459, Y456F)/IL2Rb (333-551) | IL7Ra (376- | IL2Rb (333- |
| | 416, 424- | 551) |
| | 459, | |
| | Y456F) | |
| IL7Ra (316-459)/IL2Rbsmall (393-433, 518-551) | IL7Ra (316- | IL2Rbsmall |
| | 459) | (393-433, |
| | | 518-551) |
| IL7Ra (376-416, 424-459, Y456F)/IL2Rbsmall (393- | IL7Ra (376- | IL2Rbsmall |
| 433, 518-551) | 416, 424- | (393-433, |
| | 459, | 518-551) |
| | Y456F) | |
| IL7Ra (316-459)/IL2Rbsmall (339-379, 393-433, 518- | IL7Ra (316- | IL2Rbsmall |
| 551) | 459) | (339-379, |
| | | 393-433, |
| | | 518-551) |
| IL7Ra (376-416, 424-459, Y456F)/IL2Rbsmall (339- | IL7Ra (376- | IL2Rbsmall |
| 379, 393-433, 518-551) | 416, 424- | (339-379, |
| | 459, | 393-433, |
| | Y456F) | 518-551) |
| IL2Rb (333-551)/IL7Ra (316-459) | IL2Rb (333- | IL7Ra (316- |
| | 551) | 459) |
| IL2Rb (333-551)/IL7Ra (376-416, | IL2Rb (333- | IL7Ra (376- |
| 424-459, Y456F) | 551) | 416, 424- |
| | | 459, |
| | | Y456F) |
| IL2Rbsmall (393-433, 518-551)/IL7Ra (316-459) | IL2Rbsmall | IL7Ra (316- |
| | (393-433, | 459) |
| | 518-551) | |
| IL2Rbsmall (393-433, 518-551)/ | IL2Rbsmall | IL7Ra (376- |
| IL7Ra (376-416, 424-459, Y456F) | (393-433, | 416, 424- |
| | 518-551) | 459, |
| | | Y456F) |
| IL2Rbsmall (339-379, 393-433, 518-551)/IL7Ra (316- | IL2Rbsmall | IL7Ra (316- |
| 459) | (339-379, | 459) |
| | 393-433, | |
| | 518-551) | |
| IL2Rbsmall (339-379, 393-433, 518-551)/ | IL2Rbsmall | IL7Ra (376- |
| IL7Ra (376-416, 424-459, Y456F) | (339-379, | 416, 424- |
| | 393-433, | 459, |
| | 518-551) | Y456F) |
| IL12Rb1 (622-662)/IL21R (322-538) | IL12Rb1 (622- | IL21R (322- |
| | 662) | 538) |
| IL12Rb2 (714-862)/IL21R (322-538) | IL12Rb2 (714- | IL21R (322- |
| | 862) | 538) |
| IL12Rb2 (775-825)/IL21R (322-538) | IL12Rb2 (775- | IL21R (322- |
| | 825) | 538) |
| IL21R (322-538)/IL12Rb1 (622-662) | IL21R (322- | IL12Rb1 (622- |
| | 538) | 662) |
| IL21R (322-538)/IL12Rb2 (714-862) | IL21R (322- | IL12Rb2 (714- |
| | 538) | 862) |
| IL21R (322-538)/IL12Rb2 (775-825) | IL21R (322- | IL12Rb2 (775- |
| | 538) | 825) |

Without being bound to theory or mechanism, in some embodiments, a JAK-protein (JAK1, JAK2, JAK3, or TYK2) is bound to a dimerized CACCR of the disclosure. The two bound JAK-proteins are activated, which are capable of phosphorylating tyrosine residues on the recruiting domain of the CACCR. The phosphorylated recruiting domains are then capable of binding the recruited proteins (e.g. a phosphorylated STAT-recruiting domain binds a STAT-protein), which in turn effectuate transcription events in the nucleus.

### D. Exemplary CACCRs

Table 3 shows exemplary CACCR sequences of the disclosure. The receptors may be expressed with a signal sequence, e.g. a CD8SS of sequence MALPVTALLLPLALLLHAARP (SEQ ID NO: 89).

In some embodiments, the CACCR of the disclosure comprises any one of the sequences in Table 3. In some embodiments, the CACCR comprises an amino acid sequence that is at least about 80%, 85%, 90%, 95%, 96%, 98%, 99% or 100% identical to any one of the amino acid sequences of SEQ ID NO: 90-98, and 107-139. In some embodiments, the TPOR/MPLR receptor comprises any one of the amino acid sequences of SEQ ID NO: 90-98, and 107-139.

In some embodiments, the CACCR comprises the transmembrane domain and/or JAK-binding domain derived from the TPOR/MPLR receptor. In some embodiments, the CACCR of the disclosure comprises amino acids 478-582 of the naturally occurring TPOR/MPLR receptor of SEQ ID NO: 6. In some embodiments, the CACCR of the disclosure comprises the amino acid sequence that is at least about 80%, 85%, 90%, 95%, 96%, 98%, 99% or 100% identical to the amino acid sequence of SEQ ID NO: 17. In some embodiments, the CACCR of the disclosure comprises the amino acid sequence of SEQ ID NO: 17. In some embodiments, the CACCR further comprises a recruiting domain comprising the amino acid sequence of one or more of the receptor sequences presented in Table 2b. In some embodiments, the CACCR further comprises one or more recruiting domains selected from the group consisting of the STAT-recruiting domains from IL7Ra, IL2Rb, IL12Rb1, IL12Rb2, and IL21R. In some embodiments, the recruiting domain comprises the STAT-recruiting domain from IL7Ra. In some embodiments, the STAT-recruiting domain from IL7Ra comprises the amino acid sequence that is at least about 80%, 85%, 90%, 95%, 96%, 98%, 99% or 100% identical to the amino acid sequence of SEQ ID NO: 46, 68, 69, 70, 71 or 72. In some embodiments, the STAT-recruiting domain from IL7Ra comprises the amino acid sequence of SEQ ID NO: 46, 68, 69, 70, 71 or 72. In some embodiments, the recruiting domain comprises the STAT-recruiting domain from IL2Rb. In some embodiments, the STAT-recruiting domain from IL2Rb comprises the amino acid sequence that is at least about 80%, 85%, 90%, 95%, 96%, 98%, 99% or 100% identical to the amino acid sequence of SEQ ID NO: 47, 73, 74, 75, 76, 77, 78, 106, or 143. In some embodiments, the STAT-recruiting domain from IL2Rb comprises the amino acid sequence of SEQ ID NO: 47, 73, 74, 75, 76, 77, 78, 106 or 143. In some embodiments, the recruiting domain comprises the STAT-recruiting domain from IL12Rb1 or IL12Rb2. In some embodiments, the STAT-recruiting domain from IL12Rb1 or IL12Rb2 comprises the amino acid sequence that is at least about 80%, 85%, 90%, 95%, 96%, 98%, 99% or 100% identical to the amino acid sequence of SEQ ID NO: 67, 86, or 87. In some embodiments, the STAT-recruiting domain from IL12Rb1 or IL12Rb2 comprises the amino acid sequence of SEQ ID NO: 67, 86, or 87. In some embodiments, the recruiting domain comprises the STAT-recruiting domain from IL21R. In some embodiments, the STAT-recruiting domain from IL21R comprises the amino acid sequence that is at least about 80%, 85%, 90%, 95%, 96%, 98%, 99% or 100% identical to the amino acid sequence of SEQ ID NO: 54. In some embodiments, the STAT-recruiting domain from IL21R comprises the amino acid sequence of SEQ ID NO: 54. In some embodiments, the CACCR comprises one or more recruiting domains presented in Table 2c. In some embodiments, the recruiting domains comprises the STAT-recruiting domains from IL7Ra and IL2Rb. In some embodiments, the recruiting domain comprises the STAT-recruiting domains from IL7Ra and IL12Rb1. In some embodiments, the recruiting domain comprises the STAT-recruiting domains form IL7Ra and IL12Rb2. In some embodiments, the CACCR comprises the amino acid sequence that is at least about 80%, 85%, 90%, 95%, 96%, 98%, 99% or 100% identical to the amino acid sequence of SEQ ID NO: 90 or 119, with or without a signal sequence. In some embodiments, the CACCR comprises the amino acid sequence of SEQ ID NO: 90 or 119, with or without a signal sequence.

In some embodiments, the CACCR of the disclosure comprises the transmembrane domain and/or JAK-binding domain from a TPOR/MPLR receptor that comprises one or more amino acid substitutions at H499, S505, G509 or W515. In some embodiments, the TPOR/MPLR receptor comprises a H499L substitution. In some embodiments, the TPOR/MPLR receptor comprises a S505N substitution. In some embodiments, the TPOR/MPLR receptor comprises a G509N substitution. In some embodiments, the TPOR/MPLR receptor comprises a W515K substitution. In some embodiments, the CACCR further comprises a recruiting domain comprising the amino acid sequence of one or more of the receptor sequences presented in Table 2b. In some embodiments, the CACCR further comprises one or more recruiting domains selected from the group consisting of the STAT-recruiting domains from IL7Ra, IL2Rb, IL12Rb1, IL12Rb2, and IL21R. In some embodiments, the recruiting domain comprises the STAT-recruiting domain from IL7Ra. In some embodiments, the STAT-recruiting domain from IL7Ra comprises the amino acid sequence that is at least about 80%, 85%, 90%, 95%, 96%, 98%, 99% or 100% identical to the amino acid sequence of SEQ ID NO: 46, 68, 69, 70, 71 or 72. In some embodiments, the STAT-recruiting domain from IL7Ra comprises the amino acid sequence of SEQ ID NO: 46, 68, 69, 70, 71 or 72. In some embodiments, the recruiting domain comprises the STAT-recruiting domain from IL2Rb. In some embodiments, the STAT-recruiting domain from IL2Rb comprises the amino acid sequence that is at least about 80%, 85%, 90%, 95%, 96%, 98%, 99% or 100% identical to the amino acid sequence of SEQ ID NO: 47, 73, 74, 75, 76, 77, 78, 106, or 143. In some embodiments, the STAT-recruiting domain from IL2Rb comprises the amino acid sequence of SEQ ID NO: 47, 73, 74, 75, 76, 77, 78, 106 or 143. In some embodiments, the recruiting domain comprises the STAT-recruiting domain from IL12Rb1 or IL12Rb2. In some embodiments, the STAT-recruiting domain from IL12Rb1 or IL12Rb2 comprises the amino acid sequence that is at least about 80%, 85%, 90%, 95%, 96%, 98%, 99% or 100% identical to the amino acid sequence of SEQ ID NO: 67, 86, or 87. In some embodiments, the STAT-recruiting domain from IL12Rb1 or IL12Rb2 comprises the amino acid sequence of SEQ ID NO: 67, 86, or 87. In some embodiments, the recruiting domain comprises the STAT-recruiting domain from IL21R. In some embodiments, the STAT-recruiting domain from IL21R comprises the amino acid sequence that is at least about 80%, 85%, 90%, 95%, 96%, 98%, 99% or 100% identical to the amino acid sequence of SEQ ID NO: 54. In some embodiments, the STAT-recruiting domain from IL21R comprises the amino acid sequence of SEQ ID NO: 54. In some embodiments, the CACCR comprises one or more recruiting domains presented in Table 2c. In some embodiments, the recruiting domains comprises the STAT-recruiting domains from IL7Ra and IL2Rb. In some embodiments, the recruiting domain comprises the STAT-recruiting domains from IL7Ra and IL12Rb1. In some embodiments, the recruiting domain comprises the STAT-recruiting domains form IL7Ra and IL12Rb2. In some embodiments, the CACCR comprises the amino acid sequence that is at least about 80%, 85%, 90%, 95%, 96%, 98%, 99% or 100% identical to the amino acid sequence of SEQ ID NO: 92, 94, 121, or 123, with or without a signal sequence. In some embodiments, the CACCR comprises the amino acid sequence of SEQ ID NO: 92, 94, 121, or 123, with or without a signal sequence.

In some embodiments, the CACCR of the disclosure comprises the transmembrane domain and/or JAK-binding domain from a TPOR/MPLR receptor that comprises the H499L and S505N substitutions. In some embodiments, the CACCR further comprises a recruiting domain comprising the amino acid sequence of one or more of the receptor sequences presented in Table 2b. In some embodiments, the CACCR further comprises one or more recruiting domains selected from the group consisting of the STAT-recruiting domains from IL7Ra, IL2Rb, IL12Rb1, IL12Rb2, and IL21R. In some embodiments, the recruiting domain comprises the STAT-recruiting domain from IL7Ra. In some embodiments, the STAT-recruiting domain from IL7Ra comprises the amino acid sequence that is at least about 80%, 85%, 90%, 95%, 96%, 98%, 99% or 100% identical to the amino acid sequence of SEQ ID NO: 46, 68, 69, 70, 71 or 72. In some embodiments, the STAT-recruiting domain from IL7Ra comprises the amino acid sequence of SEQ ID NO: 46, 68, 69, 70, 71 or 72. In some embodiments, the recruiting domain comprises the STAT-recruiting domain from IL2Rb. In some embodiments, the STAT-recruiting domain from IL2Rb comprises the amino acid sequence that is at least about 80%, 85%, 90%, 95%, 96%, 98%, 99% or 100% identical to the amino acid sequence of SEQ ID NO: 47, 73, 74, 75, 76, 77, 78, 106, or 143. In some embodiments, the STAT-recruiting domain from IL2Rb comprises the amino acid sequence of SEQ ID NO: 47, 73, 74, 75, 76, 77, 78, 106 or 143. In some embodiments, the recruiting domain comprises the STAT-recruiting domain from IL12Rb1 or IL12Rb2. In some embodiments, the STAT-recruiting domain from IL12Rb1 or IL12Rb2 comprises the amino acid sequence that is at least about 80%, 85%, 90%, 95%, 96%, 98%, 99% or 100% identical to the amino acid sequence of SEQ ID NO: 67, 86, or 87. In some embodiments, the STAT-recruiting domain from IL12Rb1 or IL12Rb2 comprises the amino acid sequence of SEQ ID NO: 67, 86, or 87. In some embodiments, the recruiting domain comprises the STAT-recruiting domain from IL21R. In some embodiments, the STAT-recruiting domain from IL21R comprises the amino acid sequence that is at least about 80%, 85%, 90%, 95%, 96%, 98%, 99% or 100% identical to the amino acid sequence of SEQ ID NO: 54. In some embodiments, the STAT-recruiting domain from IL21R comprises the amino acid sequence of SEQ ID NO: 54. In some embodiments, the CACCR comprises one or more recruiting domains presented in Table 2c. In some embodiments, the recruiting domains comprises the STAT-recruiting domains from IL7Ra and IL2Rb. In some embodiments, the recruiting domain comprises the STAT-recruiting domains from IL7Ra and IL12Rb1. In some embodiments, the recruiting domain comprises the STAT-recruiting domains form IL7Ra and IL12Rb2. In some embodiments, the CACCR comprises the amino acid sequence that is at least about 80%, 85%, 90%, 95%, 96%, 98%, 99% or 100% identical to the amino acid sequence of SEQ ID NO: 91, 98, 120, or 127, with or without a signal sequence. In some embodiments, the CACCR comprises the amino acid sequence of SEQ ID NO: 91, 98, 120, or 127, with or without a signal sequence.

In some embodiments, the CACCR of the disclosure comprises the transmembrane domain and/or JAK-binding domain from a TPOR/MPLR receptor that comprises the H499L and W515K substitutions or the H499L and G509N substitutions. In some embodiments, the CACCR further comprises a recruiting domain comprising the amino acid sequence of one or more of the receptor sequences presented in Table 2b. In some embodiments, the CACCR further comprises one or more recruiting domains selected from the group consisting of the STAT-recruiting domains from IL7Ra, IL2Rb, IL12Rb1, IL12Rb2, and IL21R. In some embodiments, the recruiting domain comprises the STAT-recruiting domain from IL7Ra. In some embodiments, the STAT-recruiting domain from IL7Ra comprises the amino acid sequence that is at least about 80%, 85%, 90%, 95%, 96%, 98%, 99% or 100% identical to the amino acid sequence of SEQ ID NO: 46, 68, 69, 70, 71 or 72. In some embodiments, the STAT-recruiting domain from IL7Ra comprises the amino acid sequence of SEQ ID NO: 46, 68, 69, 70, 71 or 72. In some embodiments, the recruiting domain comprises the STAT-recruiting domain from IL2Rb. In some embodiments, the STAT-recruiting domain from IL2Rb comprises the amino acid sequence that is at least about 80%, 85%, 90%, 95%, 96%, 98%, 99% or 100% identical to the amino acid sequence of SEQ ID NO: 47, 73, 74, 75, 76, 77, 78, 106, or 143. In some embodiments, the STAT-recruiting domain from IL2Rb comprises the amino acid sequence of SEQ ID NO: 47, 73, 74, 75, 76, 77, 78, 106 or 143. In some embodiments, the recruiting domain comprises the STAT-recruiting domain from IL12Rb1 or IL12Rb2. In some embodiments, the STAT-recruiting domain from IL12Rb1 or IL12Rb2 comprises the amino acid sequence that is at least about 80%, 85%, 90%, 95%, 96%, 98%, 99% or 100% identical to the amino acid sequence of SEQ ID NO: 67, 86, or 87. In some embodiments, the STAT-recruiting domain from IL12Rb1 or IL12Rb2 comprises the amino acid sequence of SEQ ID NO: 67, 86, or 87. In some embodiments, the recruiting domain comprises the STAT-recruiting domain from IL21R. In some embodiments, the STAT-recruiting domain from IL21R comprises the amino acid sequence that is at least about 80%, 85%, 90%, 95%, 96%, 98%, 99% or 100% identical to the amino acid sequence of SEQ ID NO: 54. In some embodiments, the STAT-recruiting domain from IL21R comprises the amino acid sequence of SEQ ID NO: 54. In some embodiments, the CACCR comprises one or more recruiting domains presented in Table 2c. In some embodiments, the recruiting domains comprises the STAT-recruiting domains from IL7Ra and IL2Rb. In some embodiments, the recruiting domain comprises the STAT-recruiting domains from IL7Ra and IL12Rb1. In some embodiments, the recruiting domain comprises the STAT-recruiting domains form IL7Ra and IL12Rb2. In some embodiments, the CACCR comprises the amino acid sequence that is at least about 80%, 85%, 90%, 95%, 96%, 98%, 99% or 100% identical to the amino acid sequence of SEQ ID NO: 97, or 126, with or without a signal sequence. In some embodiments, the CACCR comprises the amino acid sequence of SEQ ID NO: 97, or 126, with or without a signal sequence.

In some embodiments, the CACCR of the disclosure comprises the transmembrane domain and/or JAK-binding domain from a TPOR/MPLR receptor that comprises the S505N and W515K substitutions. In some embodiments, the CACCR further comprises a recruiting domain comprising the amino acid sequence of one or more of the receptor sequences presented in Table 2b. In some embodiments, the CACCR further comprises one or more recruiting domains selected from the group consisting of the STAT-recruiting domains from IL7Ra, IL2Rb, IL12Rb1, IL12Rb2, and IL21R. In some embodiments, the recruiting domain comprises the STAT-recruiting domain from IL7Ra. In some embodiments, the STAT-recruiting domain from IL7Ra comprises the amino acid sequence that is at least about 80%, 85%, 90%, 95%, 96%, 98%, 99% or 100% identical to the amino acid sequence of SEQ ID NO: 46, 68, 69, 70, 71 or 72. In some embodiments, the STAT-recruiting domain from IL7Ra comprises the amino acid sequence of SEQ ID NO: 46, 68, 69, 70, 71 or 72. In some embodiments, the recruiting domain comprises the STAT-recruiting domain from IL2Rb. In some embodiments, the STAT-recruiting domain from IL2Rb comprises the amino acid sequence that is at least about 80%, 85%, 90%, 95%, 96%, 98%, 99% or 100% identical to the amino acid sequence of SEQ ID NO: 47, 73, 74, 75, 76, 77, 78, 106, or 143. In some embodiments, the STAT-recruiting domain from IL2Rb comprises the amino acid sequence of SEQ ID NO: 47, 73, 74, 75, 76, 77, 78, 106 or 143. In some embodiments, the recruiting domain comprises the STAT-recruiting domain from IL12Rb1 or IL12Rb2. In some embodiments, the STAT-recruiting domain from IL12Rb1 or IL12Rb2 comprises the amino acid sequence that is at least about 80%, 85%, 90%, 95%, 96%, 98%, 99% or 100% identical to the amino acid sequence of SEQ ID NO: 67, 86, or 87. In some embodiments, the STAT-recruiting domain from IL12Rb1 or IL12Rb2 comprises the amino acid sequence of SEQ ID NO: 67, 86, or 87. In some embodiments, the recruiting domain comprises the STAT-recruiting domain from IL21R. In some embodiments, the STAT-recruiting domain from IL21R comprises the amino acid sequence that is at least about 80%, 85%, 90%, 95%, 96%, 98%, 99% or 100% identical to the amino acid sequence of SEQ ID NO: 54. In some embodiments, the STAT-recruiting domain from IL21R comprises the amino acid sequence of SEQ ID NO: 54. In some embodiments, the CACCR comprises one or more recruiting domains presented in Table 2c. In some embodiments, the recruiting domains comprises the STAT-recruiting domains from IL7Ra and IL2Rb. In some embodiments, the recruiting domain comprises the STAT-recruiting domains from IL7Ra and IL12Rb1. In some embodiments, the recruiting domain comprises the STAT-recruiting domains form IL7Ra and IL12Rb2. In some embodiments, the CACCR comprises the amino acid sequence that is at least about 80%, 85%, 90%, 95%, 96%, 98%, 99% or 100% identical to the amino acid sequence of SEQ ID NO: 96, 107, 109, 111, 113, 115, 117, 125, 128, 129, 132, 134, 136, or 138, with or without a signal sequence. In some embodiments, the CACCR comprises the amino acid sequence of SEQ ID NO: 96, 107, 109, 111, 113, 115, 117, 125, 128, 129, 132, 134, 136, or 138, with or without a signal sequence.

In some embodiments, the CACCR of the disclosure comprises the transmembrane domain and/or JAK-binding domain from a TPOR/MPLR receptor that comprises the H499L and W515K substitutions. In some embodiments, the CACCR further comprises a recruiting domain comprising the amino acid sequence of one or more of the receptor sequences presented in Table 2b. In some embodiments, the CACCR further comprises one or more recruiting domains selected from the group consisting of the STAT-recruiting domains from IL7Ra, IL2Rb, IL12Rb1, IL12Rb2, and IL21R. In some embodiments, the recruiting domain comprises the STAT-recruiting domain from IL7Ra. In some embodiments, the STAT-recruiting domain from IL7Ra comprises the amino acid sequence that is at least about 80%, 85%, 90%, 95%, 96%, 98%, 99% or 100% identical to the amino acid sequence of SEQ ID NO: 46, 68, 69, 70, 71 or 72. In some embodiments, the STAT-recruiting domain from IL7Ra comprises the amino acid sequence of SEQ ID NO: 46, 68, 69, 70, 71 or 72. In some embodiments, the recruiting domain comprises the STAT-recruiting domain from IL2Rb. In some embodiments, the STAT-recruiting domain from IL2Rb comprises the amino acid sequence that is at least about 80%, 85%, 90%, 95%, 96%, 98%, 99% or 100% identical to the amino acid sequence of SEQ ID NO: 47, 73, 74, 75, 76, 77, 78, 106, or 143. In some embodiments, the STAT-recruiting domain from IL2Rb comprises the amino acid sequence of SEQ ID NO: 47, 73, 74, 75, 76, 77, 78, 106 or 143. In some embodiments, the recruiting domain comprises the STAT-recruiting domain from IL12Rb1 or IL12Rb2. In some embodiments, the STAT-recruiting domain from IL12Rb1 or IL12Rb2 comprises the amino acid sequence that is at least about 80%, 85%, 90%, 95%, 96%, 98%, 99% or 100% identical to the amino acid sequence of SEQ ID NO: 67, 86, or 87. In some embodiments, the STAT-recruiting domain from IL12Rb1 or IL12Rb2 comprises the amino acid sequence of SEQ ID NO: 67, 86, or 87. In some embodiments, the recruiting domain comprises the STAT-recruiting domain from IL21R. In some embodiments, the STAT-recruiting domain from IL21R comprises the amino acid sequence that is at least about 80%, 85%, 90%, 95%, 96%, 98%, 99% or 100% identical to the amino acid sequence of SEQ ID NO: 54. In some embodiments, the STAT-recruiting domain from IL21R comprises the amino acid sequence of SEQ ID NO: 54. In some embodiments, the CACCR comprises one or more recruiting domains presented in Table 2c. In some embodiments, the recruiting domains comprises the STAT-recruiting domains from IL7Ra and IL2Rb. In some embodiments, the recruiting domain comprises the STAT-recruiting domains from IL7Ra and IL12Rb1. In some embodiments, the recruiting domain comprises the STAT-recruiting domains form IL7Ra and IL12Rb2. In some embodiments, the CACCR comprises the amino acid sequence that is at least about 80%, 85%, 90%, 95%, 96%, 98%, 99% or 100% identical to the amino acid sequence of SEQ ID NO: 93, with or without a signal sequence. In some embodiments, the CACCR comprises the amino acid sequence of SEQ ID NO: 93, with or without a signal sequence.

In some embodiments, the CACCR of the disclosure comprises the transmembrane domain and/or JAK-binding domain from a TPOR/MPLR receptor that comprises the H499L, S505N and W515K substitutions. In some embodiments, the CACCR further comprises a recruiting domain comprising the amino acid sequence of one or more of the receptor sequences presented in Table 2b. In some embodiments, the CACCR further comprises one or more recruiting domains selected from the group consisting of the STAT-recruiting domains from IL7Ra, IL2Rb, IL12Rb1, IL12Rb2, and IL21R. In some embodiments, the recruiting domain comprises the STAT-recruiting domain from IL7Ra. In some embodiments, the STAT-recruiting domain from IL7Ra comprises the amino acid sequence that is at least about 80%, 85%, 90%, 95%, 96%, 98%, 99% or 100% identical to the amino acid sequence of SEQ ID NO: 46, 68, 69, 70, 71 or 72. In some embodiments, the STAT-recruiting domain from IL7Ra comprises the amino acid sequence of SEQ ID NO: 46, 68, 69, 70, 71 or 72. In some embodiments, the recruiting domain comprises the STAT-recruiting domain from IL2Rb. In some embodiments, the STAT-recruiting domain from IL2Rb comprises the amino acid sequence that is at least about 80%, 85%, 90%, 95%, 96%, 98%, 99% or 100% identical to the amino acid sequence of SEQ ID NO: 47, 73, 74, 75, 76, 77, 78, 106, or 143. In some embodiments, the STAT-recruiting domain from IL2Rb comprises the amino acid sequence of SEQ ID NO: 47, 73, 74, 75, 76, 77, 78, 106 or 143. In some embodiments, the recruiting domain comprises the STAT-recruiting domain from IL12Rb1 or IL12Rb2. In some embodiments, the STAT-recruiting domain from IL12Rb1 or IL12Rb2 comprises the amino acid sequence that is at least about 80%, 85%, 90%, 95%, 96%, 98%, 99% or 100% identical to the amino acid sequence of SEQ ID NO: 67, 86, or 87. In some embodiments, the STAT-recruiting domain from IL12Rb1 or IL12Rb2 comprises the amino acid sequence of SEQ ID NO: 67, 86, or 87. In some embodiments, the recruiting domain comprises the STAT-recruiting domain from IL21R. In some embodiments, the STAT-recruiting domain from IL21R comprises the amino acid sequence that is at least about 80%, 85%, 90%, 95%, 96%, 98%, 99% or 100% identical to the amino acid sequence of SEQ ID NO: 54. In some embodiments, the STAT-recruiting domain from IL21R comprises the amino acid sequence of SEQ ID NO: 54. In some embodiments, the CACCR comprises one or more recruiting domains presented in Table 2c. In some embodiments, the recruiting domains comprises the STAT-recruiting domains from IL7Ra and IL2Rb. In some embodiments, the recruiting domain comprises the STAT-recruiting domains from IL7Ra and IL12Rb1. In some embodiments, the recruiting domain comprises the STAT-recruiting domains form IL7Ra and IL12Rb2. In some embodiments, the CACCR comprises the amino acid sequence that is at least about 80%, 85%, 90%, 95%, 96%, 98%, 99% or 100% identical to the amino acid sequence of SEQ ID NO: 95, 108, 110, 112, 114, 116, 118, 124, 130, 131, 133, 135, 137, or 139, with or without a signal sequence. In some embodiments, the CACCR comprises the amino acid sequence of SEQ ID NO: 95, 108, 110, 112, 114, 116, 118, 124, 130, 131, 133, 135, 137, or 139, with or without a signal sequence.

TABLE 3

| | | SEQ |
|---|---|---|
| Receptor | Amino acid sequence | ID NO: |
| TPOR/MPLR(478- | SDPTRVETATETAWISLVTALHLVLGLSAVLGLL | 90 |
| 582). IL7Ra(316-459) | LLRWQFPAHYRRLRHALWPSLPDLHRVLGQYLR | |
| | DTAALSPPKATVSDTCEEVEPSLLEILPKSSERTPL | |
| | PL | |
| | ARDEVEGFLQDTFPQQLEESEKQRLGGDVQSPN | |
| | CPSEDVVITPESFGRDSSLTCLAGNVSACDAPILS | |
| | SSRSLDCRESGKNGPHVYQDLLLSLGTTNSTLPPP | |
| | FSLQSGILTLNPVAQGQPILTSLGSNQEEAYVTMS | |
| | SFYQNQ | |
| TPOR/MPLR(478-582; | SDPTRVETATETAWISLVTALLLVLGLNAVLGLL | 91 |
| H499L, S505N). IL7Ra | LLRWQFPAHYRRLRHALWPSLPDLHRVLGQYLR | |
| (316-459) | DTAALSPPKATVSDTCEEVEPSLLEILPKSSERTPL | |
| | PL | |
| | ARDEVEGFLQDTFPQQLEESEKQRLGGDVQSPN | |
| | CPSEDVVITPESFGRDSSLTCLAGNVSACDAPILS | |
| | SSRSLDCRESGKNGPHVYQDLLLSLGTTNSTLPPP | |
| | FSLQSGILTLNPVAQGQPILTSLGSNQEEAYVTMS | |
| | SFYQNQ | |
| TPOR/MPLR(478- | SDPTRVETATETAWISLVTALHLVLGLNAVLGLL | 92 |
| 582; S505N). IL7Ra(316- | LLRWQFPAHYRRLRHALWPSLPDLHRVLGQYLR | |
| 459) | DTAALSPPKATVSDTCEEVEPSLLEILPKSSERTPL | |
| | PL | |
| | ARDEVEGFLQDTFPQQLEESEKQRLGGDVQSPN | |
| | CPSEDVVITPESFGRDSSLTCLAGNVSACDAPILS | |
| | SSRSLDCRESGKNGPHVYQDLLLSLGTTNSTLPPP | |
| | FSLQSGILTLNPVAQGQPILTSLGSNQEEAYVTMS | |
| | SFYQNQ | |
| TPOR/MPLR(478- | SDPTRVETATETAWISLVTALLLVLGLSAVLGLL | 93 |
| 582; H499L, W515K). | LLRKQFPAHYRRLRHALWPSLPDLHRVLGQYLR | |
| IL7Ra(316-459) | DTAALSPPKATVSDTCEEVEPSLLEILPKSSERTPL | |
| | PL | |
| | ARDEVEGFLQDTFPQQLEESEKQRLGGDVQSPN | |
| | CPSEDVVITPESFGRDSSLTCLAGNVSACDAPILS | |
| | SSRSLDCRESGKNGPHVYQDLLLSLGTTNSTLPPP | |
| | FSLQSGILTLNPVAQGQPILTSLGSNQEEAYVTMS | |
| | SFYQNQ | |
| TPOR/MPLR(478- | SDPTRVETATETAWISLVTALHLVLGLSAVLGLL | 94 |
| 582; W515K). IL7Ra(316- | LLRKQFPAHYRRLRHALWPSLPDLHRVLGQYLR | |
| 459) | DTAALSPPKATVSDTCEEVEPSLLEILPKSSERTPL | |
| | PL | |
| | ARDEVEGFLQDTFPQQLEESEKQRLGGDVQSPN | |
| | CPSEDVVITPESFGRDSSLTCLAGNVSACDAPILS | |
| | SSRSLDCRESGKNGPHVYQDLLLSLGTTNSTLPPP | |
| | FSLQSGILTLNPVAQGQPILTSLGSNQEEAYVTMS | |
| | SFYQNQ | |
| TPOR/MPLR(478- | SDPTRVETATETAWISLVTALLLVLGLNAVLGLL | 95 |
| 582; H499L, S505N, W515K). | LLRKQFPAHYRRLRHALWPSLPDLHRVLGQYLR | |
| IL7Ra(316-459) | DTAALSPPKATVSDTCEEVEPSLLEILPKSSERTPL | |
| | PL | |
| | ARDEVEGFLQDTFPQQLEESEKQRLGGDVQSPN | |
| | CPSEDVVITPESFGRDSSLTCLAGNVSACDAPILS | |
| | SSRSLDCRESGKNGPHVYQDLLLSLGTTNSTLPPP | |
| | FSLQSGILTLNPVAQGQPILTSLGSNQEEAYVTMS | |
| | SFYQNQ | |
| TPOR/MPLR(478- | SDPTRVETATETAWISLVTALHLVLGLNAVLGLL | 96 |
| 582; S505N, W515K). IL7Ra | LLRKQFPAHYRRLRHALWPSLPDLHRVLGQYLR | |
| (316-459) | DTAALSPPKATVSDTCEEVEPSLLEILPKSSERTPL | |
| | PL | |
| | ARDEVEGFLQDTFPQQLEESEKQRLGGDVQSPN | |
| | CPSEDVVITPESFGRDSSLTCLAGNVSACDAPILS | |
| | SSRSLDCRESGKNGPHVYQDLLLSLGTTNSTLPPP | |
| | FSLQSGILTLNPVAQGQPILTSLGSNQEEAYVTMS | |
| | SFYQNQ | |
| TPOR/MPLR(478- | SDPTRVETATETAWISLVTALLLVLGLSAVLNLL | 97 |
| 582; H499L, G509N). | LLRWQFPAHYRRLRHALWPSLPDLHRVLGQYLR | |
| IL7Ra(316-459) | DTAALSPPKATVSDTCEEVEPSLLEILPKSSERTPL | |
| | PL | |
| | ARDEVEGFLQDTFPQQLEESEKQRLGGDVQSPN | |
| | CPSEDVVITPESFGRDSSLTCLAGNVSACDAPILS | |
| | SSRSLDCRESGKNGPHVYQDLLLSLGTTNSTLPPP | |
| | FSLQSGILTLNPVAQGQPILTSLGSNQEEAYVTMS | |
| | SFYQNQ | |
| TPOR/MPLR(478-582, | SDPTRVETATETAWISLVTALLLVLGLNAVLGLL | 98 |
| H499L, S505N). IL7Ra | LLRWQFPAHYRRLRHALWPSLPDLHRVLGQYLR | |
| (316-459) | DTAALSPPKATVSDTCEEVEPSLLEILPKSSERTPL | |
| | PL | |
| | ARDEVEGFLQDTFPQQLEESEKQRLGGDVQSPN | |
| | CPSEDVVITPESFGRDSSLTCLAGNVSACDAPILS | |
| | SSRSLDCRESGKNGPHVYQDLLLSLGTTNSTLPPP | |
| | FSLQSGILTLNPVAQGQPILTSLGSNQEEAYVTMS | |
| | SFYQNQ | |
| CD8SS- | MALPVTALLLPLALLLHAARP | 107 |
| TPOR/MPLR(478- | SDPTRVETATETAWISLVTALHLVLGLNAVLGLL | |
| 582; S505N, W515K). | LLRKQFPAHYRRLRHALWPSLPDLHRVLGQYLR | |
| IL12Rb2(714-862) | DTAALSPPKATVSDTCEEVEPSLLEILPKSSERTPL | |
| | PL | |
| | VTPVFRHPPCSNWPQREKGIQGHQASEKDMMHS | |
| | ASSPPPPRALQAESRQLVDLYKVLESRGSDPKPE | |
| | NPACPWTVLPAGDLPTHDGYLPSNIDDLPSHEAP | |
| | LADSLEELEPQHISLSVFPSSSLHPLTFSCGDKLTL | |
| | DQLKMRCDSLML | |
| CD8SS- | MALPVTALLLPLALLLHAARP | 108 |
| TPOR/MPLR(478- | SDPTRVETATETAWISLVTALLLVLGLNAVLGLL | |
| 582; H499L, S505N, W515K). | LLRKQFPAHYRRLRHALWPSLPDLHRVLGQYLR | |
| IL12Rb2(714-862) | DTAALSPPKATVSDTCEEVEPSLLEILPKSSERTPL | |
| | PL | |
| | VTPVFRHPPCSNWPQREKGIQGHQASEKDMMHS | |
| | ASSPPPPRALQAESRQLVDLYKVLESRGSDPKPE | |
| | NPACPWTVLPAGDLPTHDGYLPSNIDDLPSHEAP | |
| | LADSLEELEPQHISLSVFPSSSLHPLTFSCGDKLTL | |
| | DQLKMRCDSLML | |
| CD8SS- | MALPVTALLLPLALLLHAARP | 109 |
| TPOR/MPLR(478- | SDPTRVETATETAWISLVTALHLVLGLNAVLGLL | |
| 582; S505N, W515K). | LLRKQFPAHYRRLRHALWPSLPDLHRVLGQYLR | |
| IL12Rb2(775-825) | DTAALSPPKATVSDTCEEVEPSLLEILPKSSERTPL | |
| | PL | |
| | SDPKPENPACPWTVLPAGDLPTHDGYLPSNIDDL | |
| | PSHEAPLADSLEELEPQ | |
| CD8SS- | MALPVTALLLPLALLLHAARP | 110 |
| TPOR/MPLR(478- | SDPTRVETATETAWISLVTALLLVLGLNAVLGLL | |
| 582; H499L, S505N, W515K). | LLRKQFPAHYRRLRHALWPSLPDLHRVLGQYLR | |
| IL12Rb2(775-825) | DTAALSPPKATVSDTCEEVEPSLLEILPKSSERTPL | |
| | PL | |
| | SDPKPENPACPWTVLPAGDLPTHDGYLPSNIDDL | |
| | PSHEAPLADSLEELEPQ | |
| CD8SS- | MALPVTALLLPLALLLHAARP | 111 |
| TPOR/MPLR(478- | SDPTRVETATETAWISLVTALHLVLGLNAVLGLL | |
| 582; S505N, W515K). | LLRKQFPAHYRRLRHALWPSLPDLHRVLGQYLR | |
| IL2Rb(333-551) | DTAALSPPKATVSDTCEEVEPSLLEILPKSSERTPL | |
| | PL | |
| | VTQLLLQQDKVPEPASLSSNHSLTSCFTNQGYFF | |
| | FHLPDALEIEACQVYFTYDPYSEEDPDEGVAGAP | |
| | TGSSPQPLQPLSGEDDAYCTFPSRDDLLLFSPSLL | |
| | GGPSPPSTAPGGSGAGEERMPPSLQERVPRDWDP | |
| | QPLGPPTPGVPDLVDFQPPPELVLREAGEEVPDA | |
| | GPREGVSFPWSRPPGQGEFRALNARLPLNTDAYL | |
| | SLQELQGQDPTHLV | |
| CD8SS- | MALPVTALLLPLALLLHAARP | 112 |
| TPOR/MPLR(478- | SDPTRVETATETAWISLVTALLLVLGLNAVLGLL | |
| 582; H499L, S505N, W515K). | LLRKQFPAHYRRLRHALWPSLPDLHRVLGQYLR | |
| IL2Rb (333-551) | DTAALSPPKATVSDTCEEVEPSLLEILPKSSERTPL | |
| | PL | |
| | VTQLLLQQDKVPEPASLSSNHSLTSCFTNQGYFF | |
| | FHLPDALEIEACQVYFTYDPYSEEDPDEGVAGAP | |
| | TGSSPQPLQPLSGEDDAYCTFPSRDDLLLFSPSLL | |
| | GGPSPPSTAPGGSGAGEERMPPSLQERVPRDWDP | |
| | QPLGPPTPGVPDLVDFQPPPELVLREAGEEVPDA | |
| | GPREGVSFPWSRPPGQGEFRALNARLPLNTDAYL | |
| | SLQELQGQDPTHLV | |
| CD8SS- | MALPVTALLLPLALLLHAARP | 113 |
| TPOR/MPLR(478- | SDPTRVETATETAWISLVTALHLVLGLNAVLGLL | |
| 582; S505N, W515K). | LLRKQFPAHYRRLRHALWPSLPDLHRVLGQYLR | |
| IL2Rb(393-433, 518-551) | DTAALSPPKATVSDTCEEVEPSLLEILPKSSERTPL | |
| | PL | |
| | DEGVAGAPTGSSPQPLQPLSGEDDAYCTFPSRDD | |
| | LLLFSPSGQGEFRALNARLPLNTDAYLSLQELQG | |
| | QDPTHLV | |
| CD8SS- | MALPVTALLLPLALLLHAARP | 114 |
| TPOR/MPLR(478- | SDPTRVETATETAWISLVTALLLVLGLNAVLGLL | |
| 582; H499L, S505N, W515K) | LLRKQFPAHYRRLRHALWPSLPDLHRVLGQYLR | |
| IL2Rb(393-433, 518- | DTAALSPPKATVSDTCEEVEPSLLEILPKSSERTPL | |
| 551) | PL | |
| | DEGVAGAPTGSSPQPLQPLSGEDDAYCTFPSRDD | |
| | LLLFSPSGQGEFRALNARLPLNTDAYLSLQELQG | |
| | QDPTHLV | |
| CD8SS- | MALPVTALLLPLALLLHAARP | 115 |
| TPOR/MPLR(478- | SDPTRVETATETAWISLVTALHLVLGLNAVLGLL | |
| 582; S505N, W515K). | LLRKQFPAHYRRLRHALWPSLPDLHRVLGQYLR | |
| IL2Rb(339-379, 393- | DTAALSPPKATVSDTCEEVEPSLLEILPKSSERTPL | |
| 433, 518-551) | PL | |
| | QQDKVPEPASLSSNHSLTSCFTNQGYFFFHLPDA | |
| | LEIEACQDEGVAGAPTGSSPQPLQPLSGEDDAYC | |
| | TFPSRDDLLLFSPSGQGEFRALNARLPLNTDAYLS | |
| | LQELQGQDPTHLV | |
| CD8SS- | MALPVTALLLPLALLLHAARP | 116 |
| TPOR/MPLR(478- | SDPTRVETATETAWISLVTALLLVLGLNAVLGLL | |
| 582; H499L, S505N, W515K). | LLRKQFPAHYRRLRHALWPSLPDLHRVLGQYLR | |
| IL2Rb (339-379,393- | DTAALSPPKATVSDTCEEVEPSLLEILPKSSERTPL | |
| 433, 518-551) | PL | |
| | QQDKVPEPASLSSNHSLTSCFTNQGYFFFHLPDA | |
| | LEIEACQDEGVAGAPTGSSPQPLQPLSGEDDAYC | |
| | TFPSRDDLLLFSPSGQGEFRALNARLPLNTDAYLS | |
| | LQELQGQDPTHLV | |
| CD8SS- | MALPVTALLLPLALLLHAARP | 117 |
| TPOR/MPLR(478- | SDPTRVETATETAWISLVTALHLVLGLNAVLGLL | |
| 582; S505N, W515K). | LLRKQFPAHYRRLRHALWPSLPDLHRVLGQYLR | |
| IL7Ra(316-459). IL12Rb2 | DTAALSPPKATVSDTCEEVEPSLLEILPKSSERTPL | |
| (775-825) | PL | |
| | ARDEVEGFLQDTFPQQLEESEKQRLGGDVQSPN | |
| | CPSEDVVITPESFGRDSSLTCLAGNVSACDAPILS | |
| | SSRSLDCRESGKNGPHVYQDLLLSLGTTNSTLPPP | |
| | FSLQSGILTLNPVAQGQPILTSLGSNQEEAYVTMS | |
| | SFYQNQ | |
| | SDPKPENPACPWTVLPAGDLPTHDGYLPSNIDDL | |
| | PSHEAPLADSLEELEPQ | |
| CD8SS- | MALPVTALLLPLALLLHAARP | 118 |
| TPOR/MPLR(478- | SDPTRVETATETAWISLVTALLLVLGLNAVLGLL | |
| 582; H499L, S505N, W515K). | LLRKQFPAHYRRLRHALWPSLPDLHRVLGQYLR | |
| IL7Ra(316-459). | DTAALSPPKATVSDTCEEVEPSLLEILPKSSERTPL | |
| IL12Rb2(775-825) | PL | |
| | ARDEVEGFLQDTFPQQLEESEKQRLGGDVQSPN | |
| | CPSEDVVITPESFGRDSSLTCLAGNVSACDAPILS | |
| | SSRSLDCRESGKNGPHVYQDLLLSLGTTNSTLPPP | |
| | FSLQSGILTLNPVAQGQPILTSLGSNQEEAYVTMS | |
| | SFYQNQ | |
| | SDPKPENPACPWTVLPAGDLPTHDGYLPSNIDDL | |
| | PSHEAPLADSLEELEPQ | |
| TPOR/MPLR(478- | SDPTRVETATETAWISLVTALHLVLGLSAVLGLL | 119 |
| 582). IL7Ra(316-459) | LLRWQFPAHYRRLRHALWPSLPDLHRVLGQYLR | |
| | DTAALSPPKATVSDTCEEVEPSLLEILPKSSERTPL | |
| | PLLEARDEVEGFLQDTFPQQLEESEKQRLGGDVQ | |
| | SPNCPSEDVVITPESFGRDSSLTCLAGNVSACDAP | |
| | ILSSSRSLDCRESGKNGPHVYQDLLLSLGTTNSTL | |
| | PPPFSLQSGILTLNPVAQGQPILTSLGSNQEEAYV | |
| | TMSSFYQNQ | |
| TPOR/MPLR(478-582; | SDPTRVETATETAWISLVTALLLVLGLNAVLGLL | 120 |
| H499L, S505N). IL7Ra | LLRWQFPAHYRRLRHALWPSLPDLHRVLGQYLR | |
| (316-459) | DTAALSPPKATVSDTCEEVEPSLLEILPKSSERTPL | |
| | PLLEARDEVEGFLQDTFPQQLEESEKQRLGGDVQ | |
| | SPNCPSEDVVITPESFGRDSSLTCLAGNVSACDAP | |
| | ILSSSRSLDCRESGKNGPHVYQDLLLSLGTTNSTL | |
| | PPPFSLQSGILTLNPVAQGQPILTSLGSNQEEAYV | |
| | TMSSFYQNQ | |
| TPOR/MPLR(478- | SDPTRVETATETAWISLVTALHLVLGLNAVLGLL | 121 |
| 582; S505N). IL7Ra | LLRWQFPAHYRRLRHALWPSLPDLHRVLGQYLR | |
| (316-459) | DTAALSPPKATVSDTCEEVEPSLLEILPKSSERTPL | |
| | PLLEARDEVEGFLQDTFPQQLEESEKQRLGGDVQ | |
| | SPNCPSEDVVITPESFGRDSSLTCLAGNVSACDAP | |
| | ILSSSRSLDCRESGKNGPHVYQDLLLSLGTTNSTL | |
| | PPPFSLQSGILTLNPVAQGQPILTSLGSNQEEAYV | |
| | TMSSFYQNQ | |
| TPOR/MPLR(478- | SDPTRVETATETAWISLVTALLLVLGLSAVLGLL | 122 |
| 582; H499L, W515K). | LLRKQFPAHYRRLRHALWPSLPDLHRVLGQYLR | |
| IL7Ra(316-459) | DTAALSPPKATVSDTCEEVEPSLLEILPKSSERTPL | |
| | PLLEARDEVEGFLQDTFPQQLEESEKQRLGGDVQ | |
| | SPNCPSEDVVITPESFGRDSSLTCLAGNVSACDAP | |
| | ILSSSRSLDCRESGKNGPHVYQDLLLSLGTTNSTL | |
| | PPPFSLQSGILTLNPVAQGQPILTSLGSNQEEAYV | |
| | TMSSFYQNQ | |
| TPOR/MPLR(478- | SDPTRVETATETAWISLVTALHLVLGLSAVLGLL | 123 |
| 582; W515K). IL7Ra(316- | LLRKQFPAHYRRLRHALWPSLPDLHRVLGQYLR | |
| 459) | DTAALSPPKATVSDTCEEVEPSLLEILPKSSERTPL | |
| | PLLEARDEVEGFLQDTFPQQLEESEKQRLGGDVQ | |
| | SPNCPSEDVVITPESFGRDSSLTCLAGNVSACDAP | |
| | ILSSSRSLDCRESGKNGPHVYQDLLLSLGTTNSTL | |
| | PPPFSLQSGILTLNPVAQGQPILTSLGSNQEEAYV | |
| | TMSSFYQNQ | |
| TPOR/MPLR(478-582; | SDPTRVETATETAWISLVTALLLVLGLNAVLGLL | 124 |
| H499L, S505N, W515K). | LLRKQFPAHYRRLRHALWPSLPDLHRVLGQYLR | |
| IL7Ra(316-459) | DTAALSPPKATVSDTCEEVEPSLLEILPKSSERTPL | |
| | PLLEARDEVEGFLQDTFPQQLEESEKQRLGGDVQ | |
| | SPNCPSEDVVITPESFGRDSSLTCLAGNVSACDAP | |
| | ILSSSRSLDCRESGKNGPHVYQDLLLSLGTTNSTL | |
| | PPPFSLQSGILTLNPVAQGQPILTSLGSNQEEAYV | |
| | TMSSFYQNQ | |
| TPOR/MPLR(478-582; | SDPTRVETATETAWISLVTALHLVLGLNAVLGLL | 125 |
| S505N, W515K). | LLRKQFPAHYRRLRHALWPSLPDLHRVLGQYLR | |
| IL7Ra(316-459) | DTAALSPPKATVSDTCEEVEPSLLEILPKSSERTPL | |
| | PLLEARDEVEGFLQDTFPQQLEESEKQRLGGDVQ | |
| | SPNCPSEDVVITPESFGRDSSLTCLAGNVSACDAP | |
| | ILSSSRSLDCRESGKNGPHVYQDLLLSLGTTNSTL | |
| | PPPFSLQSGILTLNPVAQGQPILTSLGSNQEEAYV | |
| | TMSSFYQNQ | |
| TPOR/MPLR(478-582, | SDPTRVETATETAWISLVTALLLVLGLSAVLNLL | 126 |
| H499L, G509N). | LLRWQFPAHYRRLRHALWPSLPDLHRVLGQYLR | |
| IL7Ra(316-459) | DTAALSPPKATVSDTCEEVEPSLLEILPKSSERTPL | |
| | PLLEARDEVEGFLQDTFPQQLEESEKQRLGGDVQ | |
| | SPNCPSEDVVITPESFGRDSSLTCLAGNVSACDAP | |
| | ILSSSRSLDCRESGKNGPHVYQDLLLSLGTTNSTL | |
| | PPPFSLQSGILTLNPVAQGQPILTSLGSNQEEAYV | |
| | TMSSFYQNQ | |
| TPOR/MPLR(478-582; | SDPTRVETATETAWISLVTALLLVLGLNAVLGLL | 127 |
| H499L, S505N). IL7Ra | LLRWQFPAHYRRLRHALWPSLPDLHRVLGQYLR | |
| (316-459) | DTAALSPPKATVSDTCEEVEPSLLEILPKSSERTPL | |
| | PLLEARDEVEGFLQDTFPQQLEESEKQRLGGDVQ | |
| | SPNCPSEDVVITPESFGRDSSLTCLAGNVSACDAP | |
| | ILSSSRSLDCRESGKNGPHVYQDLLLSLGTTNSTL | |
| | PPPFSLQSGILTLNPVAQGQPILTSLGSNQEEAYV | |
| | TMSSFYQNQ | |
| TPOR/MPLR(478-582; | SDPTRVETATETAWISLVTALHLVLGLNAVLGLL | 128 |
| S505N, W515K). | LLRKQFPAHYRRLRHALWPSLPDLHRVLGQYLR | |
| IL2Rb(333-551) | DTAALSPPKATVSDTCEEVEPSLLEILPKSSERTPL | |
| | PLLEVTQLLLQQDKVPEPASLSSNHSLTSCFTNQ | |
| | GYFFFHLPDALEIEACQVYFTYDPYSEEDPDEGV | |
| | AGAPTGSSPQPLQPLSGEDDAYCTFPSRDDLLLFS | |
| | PSLLGGPSPPSTAPGGSGAGEERMPPSLQERVPRD | |
| | WDPQPLGPPTPGVPDLVDFQPPPELVLREAGEEV | |
| | PDAGPREGVSFPWSRPPGQGEFRALNARLPLNTD | |
| | AYLSLQELQGQDPTHLV | |
| TPOR/MPLR(478-582; | SDPTRVETATETAWISLVTALHLVLGLNAVLGLL | 129 |
| S505N, W515K). | LLRKQFPAHYRRLRHALWPSLPDLHRVLGQYLR | |
| IL12Rb2(714-862) | DTAALSPPKATVSDTCEEVEPSLLEILPKSSERTPL | |
| | PLLEVTPVFRHPPCSNWPQREKGIQGHQASEKD | |
| | MMHSASSPPPPRALQAESRQLVDLYKVLESRGS | |
| | DPKPENPACPWTVLPAGDLPTHDGYLPSNIDDLP | |
| | SHEAPLADSLEELEPQHISLSVFPSSSLHPLTFSCG | |
| | DKLTLDQLKMRCDSLML | |
| TPOR/MPLR(478-582; | SDPTRVETATETAWISLVTALLLVLGLNAVLGLL | 130 |
| H499L, S505N, | LLRKQFPAHYRRLRHALWPSLPDLHRVLGQYLR | |
| W515K). IL2Rb(333-551) | DTAALSPPKATVSDTCEEVEPSLLEILPKSSERTPL | |
| | PLLEVTQLLLQQDKVPEPASLSSNHSLTSCFTNQ | |
| | GYFFFHLPDALEIEACQVYFTYDPYSEEDPDEGV | |
| | AGAPTGSSPQPLQPLSGEDDAYCTFPSRDDLLLFS | |
| | PSLLGGPSPPSTAPGGSGAGEERMPPSLQERVPRD | |
| | WDPQPLGPPTPGVPDLVDFQPPPELVLREAGEEV | |
| | PDAGPREGVSFPWSRPPGQGEFRALNARLPLNTD | |
| | AYLSLQELQGQDPTHLV | |
| TPOR/MPLR(478- | SDPTRVETATETAWISLVTALLLVLGLNAVLGLL | 131 |
| 582; H499L, S505N, | LLRKQFPAHYRRLRHALWPSLPDLHRVLGQYLR | |
| W515K). IL12Rb2(714-862) | DTAALSPPKATVSDTCEEVEPSLLEILPKSSERTPL | |
| | PLLEVTPVFRHPPCSNWPQREKGIQGHQASEKD | |
| | MMHSASSPPPPRALQAESRQLVDLYKVLESRGS | |
| | DPKPENPACPWTVLPAGDLPTHDGYLPSNIDDLP | |
| | SHEAPLADSLEELEPQHISLSVFPSSSLHPLTFSCG | |
| | DKLTLDQLKMRCDSLML | |
| TPOR/MPLR(478-582, | SDPTRVETATETAWISLVTALHLVLGLNAVLGLL | 132 |
| S505N, W515K). | LLRKQFPAHYRRLRHALWPSLPDLHRVLGQYLR | |
| IL12Rb2(775-825) | DTAALSPPKATVSDTCEEVEPSLLEILPKSSERTPL | |
| | PLLESDPKPENPACPWTVLPAGDLPTHDGYLPSN | |
| | IDDLPSHEAPLADSLEELEPQ | |
| TPOR/MPLR(478-582; | SDPTRVETATETAWISLVTALLLVLGLNAVLGLL | 133 |
| H499L, S505N, | LLRKQFPAHYRRLRHALWPSLPDLHRVLGQYLR | |
| W515K). IL12Rb2(775-825) | DTAALSPPKATVSDTCEEVEPSLLEILPKSSERTPL | |
| | PLLESDPKPENPACPWTVLPAGDLPTHDGYLPSN | |
| | IDDLPSHEAPLADSLEELEPQ | |
| TPOR/MPLR(478-582; | SDPTRVETATETAWISLVTALHLVLGLNAVLGLL | 134 |
| S505N, W515K). | LLRKQFPAHYRRLRHALWPSLPDLHRVLGQYLR | |
| IL7Ra(316-459). IL12Rb2 | DTAALSPPKATVSDTCEEVEPSLLEILPKSSERTPL | |
| (775-825) | PLLEARDEVEGFLQDTFPQQLEESEKQRLGGDVQ | |
| | SPNCPSEDVVITPESFGRDSSLTCLAGNVSACDAP | |
| | ILSSSRSLDCRESGKNGPHVYQDLLLSLGTTNSTL | |
| | PPPFSLQSGILTLNPVAQGQPILTSLGSNQEEAYV | |
| | TMSSFYQNQSRSDPKPENPACPWTVLPAGDLPTH | |
| | DGYLPSNIDDLPSHEAPLADSLEELEPQ | |
| TPOR/MPLR(478-582; | SDPTRVETATETAWISLVTALLLVLGLNAVLGLL | 135 |
| H499L, S505N, | LLRKQFPAHYRRLRHALWPSLPDLHRVLGQYLR | |
| W515K). IL7Ra(316- | DTAALSPPKATVSDTCEEVEPSLLEILPKSSERTPL | |
| 459). IL12Rb2(775-825) | PLLEARDEVEGFLQDTFPQQLEESEKQRLGGDVQ | |
| | SPNCPSEDVVITPESFGRDSSLTCLAGNVSACDAP | |
| | ILSSSRSLDCRESGKNGPHVYQDLLLSLGTTNSTL | |
| | PPPFSLQSGILTLNPVAQGQPILTSLGSNQEEAYV | |
| | TMSSFYQNQSRSDPKPENPACPWTVLPAGDLPTH | |
| | DGYLPSNIDDLPSHEAPLADSLEELEPQ | |
| TPOR/MPLR(478-582; | SDPTRVETATETAWISLVTALHLVLGLNAVLGLL | 136 |
| S505N, W515K). IL2Rb | LLRKQFPAHYRRLRHALWPSLPDLHRVLGQYLR | |
| (333-551; Y381S, | DTAALSPPKATVSDTCEEVEPSLLEILPKSSERTPL | |
| Y384S, Y387S) | PLLEVTQLLLQQDKVPEPASLSSNHSLTSCFTNQ | |
| | GYFFFHLPDALEIEACQVSFTSDPSSEEDPDEGVA | |
| | GAPTGSSPQPLQPLSGEDDAYCTFPSRDDLLLFSP | |
| | SLLGGPSPPSTAPGGSGAGEERMPPSLQERVPRD | |
| | WDPQPLGPPTPGVPDLVDFQPPPELVLREAGEEV | |
| | PDAGPREGVSFPWSRPPGQGEFRALNARLPLNTD | |
| | AYLSLQELQGQDPTHLV | |
| TPOR/MPLR(478-582; | SDPTRVETATETAWISLVTALLLVLGLNAVLGLL | 137 |
| H499L, S505N, W515K). | LLRKQFPAHYRRLRHALWPSLPDLHRVLGQYLR | |
| IL2Rb(333-551; | DTAALSPPKATVSDTCEEVEPSLLEILPKSSERTPL | |
| Y381S, Y384S, Y387S) | PLLEVTQLLLQQDKVPEPASLSSNHSLTSCFTNQ | |
| | GYFFFHLPDALEIEACQVSFTSDPSSEEDPDEGVA | |
| | GAPTGSSPQPLQPLSGEDDAYCTFPSRDDLLLFSP | |
| | SLLGGPSPPSTAPGGSGAGEERMPPSLQERVPRD | |
| | WDPQPLGPPTPGVPDLVDFQPPPELVLREAGEEV | |
| | PDAGPREGVSFPWSRPPGQGEFRALNARLPLNTD | |
| | AYLSLQELQGQDPTHLV | |
| TPOR/MPLR(478-582; | SDPTRVETATETAWISLVTALHLVLGLNAVLGLL | 138 |
| S505N, W515K). | LLRKQFPAHYRRLRHALWPSLPDLHRVLGQYLR | |
| IL2Rb(333-551; | DTAALSPPKATVSDTCEEVEPSLLEILPKSSERTPL | |
| Y364S, Y381S, Y384S, | PLLEVTQLLLQQDKVPEPASLSSNHSLTSCFTNQ | |
| Y387S) | GSFFFHLPDALEIEACQVSFTSDPSSEEDPDEGVA | |
| | GAPTGSSPQPLQPLSGEDDAYCTFPSRDDLLLFSP | |
| | SLLGGPSPPSTAPGGSGAGEERMPPSLQERVPRD | |
| | WDPQPLGPPTPGVPDLVDFQPPPELVLREAGEEV | |
| | PDAGPREGVSFPWSRPPGQGEFRALNARLPLNTD | |
| | AYLSLQELQGQDPTHLV | |
| TPOR/MPLR(478-582; | SDPTRVETATETAWISLVTALLLVLGLNAVLGLL | 139 |
| H499L, S505N, W515K). | LLRKQFPAHYRRLRHALWPSLPDLHRVLGQYLR | |
| IL2Rb(333-551; | DTAALSPPKATVSDTCEEVEPSLLEILPKSSERTPL | |
| Y364S, Y381S, Y384S, | PLLEVTQLLLQQDKVPEPASLSSNHSLTSCFTNQ | |
| Y387S) | GSFFFHLPDALEIEACQVSFTSDPSSEEDPDEGVA | |
| | GAPTGSSPQPLQPLSGEDDAYCTFPSRDDLLLFSP | |
| | SLLGGPSPPSTAPGGSGAGEERMPPSLQERVPRD | |
| | WDPQPLGPPTPGVPDLVDFQPPPELVLREAGEEV | |
| | PDAGPREGVSFPWSRPPGQGEFRALNARLPLNTD | |
| | AYLSLQELQGQDPTHLV | |

*The underlined LE and SR are exemplary optional linker that may be inserted between two domains.

### E. Expression of CACCRs

Provided herein are polynucleotides encoding any one of the CACCRs provided herein. Likewise, provided herein are expression vectors comprising such polynucleotides. In some embodiments, the vector is a viral vector. In some embodiments, the vector is not a viral vector.

In some embodiments, the expression vector comprises a CACCR and a polynucleotide expressing a chimeric antigen receptor (CAR).

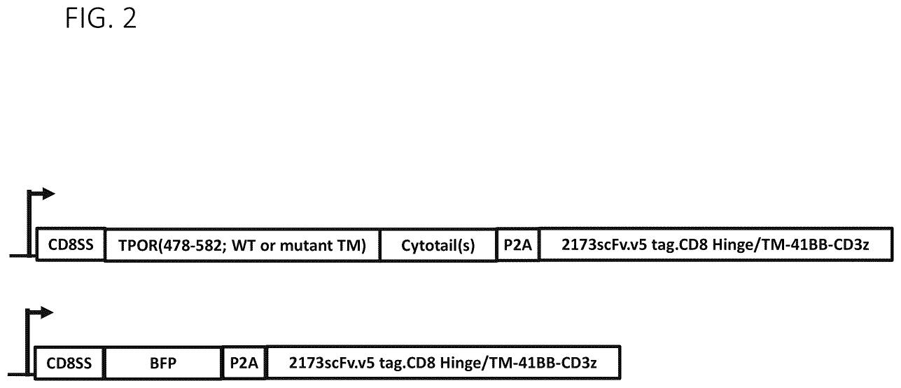

In some embodiments, expression of the CACCR and the CAR are expressed as a single polypeptide chain, separated by a linker. FIG. 2 shows a schematic of a vector that can be used to co-express the CACCR and CAR of the disclosure. One or more recruiting domains may be joined in tandem to mimic signaling from one or more cytokines.

### II. CAR-bearing Immune Cells

Provided herein are engineered immune cells comprising a polynucleotide encoding a chimeric antigen receptor (CAR) and a CACCR of the disclosure; and provided herein are engineered immune cells expressing a chimeric antigen receptor (CAR-I cell) and a CACCR of the disclosure. Examples of immune cells include T-cells, e.g., alpha/beta T-cells and gamma/delta T-cells, B cells, natural killer (NK) cells, natural killer T (NKT) cells, invariant NKT cells, mast cells, myeloic-derived phagocytes, dendritic cells, killer dendritic cells, macrophages, and monocytes. Immune cells also refer to cells derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells.

Accordingly in some embodiments, provided herein are CAR-T-cells comprising a CACCR of the disclosure.

In some embodiments, a CAR can comprise an extracellular ligand-binding domain (e.g., a single chain variable fragment (scFv)), a transmembrane domain, and an intracellular signaling domain. In some embodiments, the extracellular ligand-binding domain, transmembrane domain, and intracellular signaling domain are in one polypeptide, i.e., in a single chain. Multichain CARs and polypeptides are also provided herein. In some embodiments, the multichain CARs comprise: a first polypeptide comprising a transmembrane domain and at least one extracellular ligand-binding domain, and a second polypeptide comprising a transmembrane domain and at least one intracellular signaling domain, wherein the polypeptides assemble together to form a multichain CAR.

The extracellular ligand-binding domain of a CAR specifically binds to a target of interest. The target of interest can be any molecule of interest, including, for example without limitation BCMA, EGFRvIII, Flt-3, WT-1, CD20, CD23, CD30, CD38, CD70, CD33, CD133, LeY, NKG2D, CS1, CD44v6, ROR1, CD19, Claudin-18.2 (Claudin-18A2, or Claudin 18 isoform 2), DLL3 (Delta-like protein 3, Drosophila Delta homolog 3, Delta3), Muc17 (Mucin17, Muc3, Muc3), FAP alpha (Fibroblast Activation Protein alpha), Ly6G6D (Lymphocyte antigen 6 complex locus protein God, c6orf23, G6D, MEGT1, NG25), and/or RNF43 (E3 ubiquitin-protein ligase RNF43, RING finger protein 43).

In some embodiments, the extracellular ligand-binding domain of a CAR comprises an scFv comprising the light chain variable (VL) region and the heavy chain variable (VH) region of a target antigen specific monoclonal antibody joined by a flexible linker. Single chain variable region fragments are made by linking light and/or heavy chain variable regions by using a short linking peptide (Bird et al., Science 242:423-426, 1988) (e.g. glycine-serine containing linkers). In general, linkers can be short, flexible polypeptides and are generally comprised of about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The intracellular signaling domain of a CAR according to the invention is responsible for intracellular signaling following the binding of extracellular ligand-binding domain to the target resulting in the activation of the immune cell and immune response (Signals 1 and/or 2). The intracellular signaling domain has the ability to activate of at least one of the normal effector functions of the immune cell in which the CAR is expressed. For example, the effector function of a T cell can be a cytolytic activity or helper activity including the secretion of cytokines.

In some embodiments, an intracellular signaling domain for use in a CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability. Intracellular signaling domains comprise two distinct classes of cytoplasmic signaling sequences: those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non-limiting examples those derived from TCRζ, FcRγ, FcRβ, FcRε, CD3γ, CD3δ, CD3ε, CD5, CD22, CD79a, CD79b and CD66d. In some embodiments, the intracellular signaling domain of the CAR can comprise the CD3ζ signaling domain. In some embodiments the intracellular signaling domain of the CAR of the invention comprises a domain of a co-stimulatory molecule.

In some embodiments, the intracellular signaling domain of a CAR of the invention comprises a part of co-stimulatory molecule selected from the group consisting of fragment of 41BB (GenBank: AAA53133.) and CD28 (NP_006130.1).

CARs are expressed on the surface membrane of the cell. Thus, the CAR comprises a transmembrane domain. Suitable transmembrane domains for a CAR disclosed herein have the ability to (a) be expressed at the surface of a cell, preferably an immune cell such as, for example without limitation, lymphocyte cells or Natural killer (NK) cells, and (b) interact with the ligand-binding domain and intracellular signaling domain for directing cellular response of immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a subunit of the T cell receptor such as α, β, γ or δ, polypeptide constituting CD3 complex, IL-2 receptor p55 (a chain), p75 (β chain) or γ chain, subunit chain of Fc receptors, in particular Fcγ receptor III or CD proteins. Alternatively, the transmembrane domain can be synthetic and can comprise predominantly hydrophobic residues such as leucine and valine. In some embodiments said transmembrane domain is derived from the human CD8α chain (e.g., NP_001139345.1). The transmembrane domain can further comprise a stalk domain between the extracellular ligand-binding domain and said transmembrane domain. A stalk domain may comprise up to 300 amino acids, preferably 10 to 100 amino acids and most preferably 25 to 50 amino acids. Stalk region may be derived from all or part of naturally occurring molecules, such as from all or part of the extracellular region of CD8, CD4, or CD28, or from all or part of an antibody constant region. Alternatively the stalk domain may be a synthetic sequence that corresponds to a naturally occurring stalk sequence, or may be an entirely synthetic stalk sequence. In some embodiments said stalk domain is a part of human CD8α chain (e.g., NP_001139345.1). In another particular embodiment, said transmembrane and hinge domains comprise a part of human CD8α chain. In some embodiments, the intracellular signaling domain comprises a CD3ζ signaling domain. In some embodiments, the intracellular signaling domain comprises a CD3ζ signaling domain and additionally a second signaling domain. In some embodiments, the intracellular signaling domain comprises a CD3ζ signaling domain and a 4-1BB signaling domain. In some embodiments, CARs disclosed herein can comprise an extracellular ligand-binding domain that specifically binds BCMA or EGFRvIII, CD8α human hinge and transmembrane domains, the CD3ζ signaling domain, and 4-1BB signaling domain. In some embodiments, the EGFRvIII specific CAR comprises the amino acid sequence of SEQ ID NO: 140. In some embodiments, the BCMA specific CAR comprises the amino acid sequence of SEQ ID NO: 141 or 142, with or without a signal sequence.

In some aspects, the CAR-immune cell is a BCMA CAR-T cell comprising a CACCR of the disclosure. In some embodiments, the CACCR of the BCMA CAR-T cell comprises a transmembrane/JAK-binding domain of amino acids 478-582 of the naturally occurring TPOR/MPLR receptor of SEQ ID NO: 6, with H499L, S505N, and W515K triple substitutions, or S505N and W515K double substitutions (e.g., SEQ ID NO: 12 or 13). In some embodiments, the CACCR further comprises a recruiting domain from IL2Rb. In some embodiments, the CACCR of the BCMA CAR-T cell further comprises a recruiting domain from IL2Rb(393-433,518-551) or IL2Rb(339-379,393-433,518-551) (e.g., SEQ ID NO: 77 or 78). In some embodiments, the BCMA specific CAR comprises the amino acid sequence of SEQ ID NO: 141 or 142, with or without a signal sequence. In some embodiments, the BCMA CAR-T cells comprise a CACCR that comprises the amino acid sequence of SEQ ID NO: 113, 114, or 116, with or without a signal sequence.

In some embodiments, a CAR can be introduced into an immune cell as a transgene via a plasmid vector. In some embodiments, the plasmid vector can also contain, for example, a selection marker which provides for identification and/or selection of cells which received the vector.

Table 4 provides exemplary sequences of CAR components that can be used in the CARs disclosed herein and the antibody and/or CAR sequences exemplified herein.

TABLE 4
Sequences relating to CARs

| | | SEQ |
|---|---|---|
| Domain | Amino acid sequence | ID |
| V5 epitope tag | IPNPLLGLDST | 99 |
| 2173 scFv | EIQLVQSGAEVKKPGESLRISCKGSGFNIEDYYIH | 100 |
| | WVRQMPGKGLEWMGRIDPENDETKYGPIFQGH | |
| | VTISADTSINTVYLQWSSLKASDTAMYYCAFRG | |
| | GVYWGQGTTVTVSSGGGGSGGGGSGGGGSGGG | |
| | GSDVVMTQSPDSLAVSLGERATINCKSSQSLLDS | |
| | DGKTYLNWLQQKPGQPPKRLISLVSKLDSGVPD | |
| | RFSGSGSGTDFTLTISSLQAEDVAVYYCWQGTHF | |
| | PGTFGGGTKVEIK | |
| CD8 hinge and | TTTPAPRPPTPAPTIASQPLSLRPEACRPAAGGAV | 101 |
| transmembrane | HTRGLDFACDIYIWAPLAGTCGVLLLSLVITLYC | |
| 4-1BB intracellular | KRGRKKLLYIFKQPFMRPVQTTQEEDGCSCRFPE | 102 |
| signaling | EEEGGCEL | |
| CD3z intracellular | RVKFSRSADAPAYQQGQNQLYNELNLGRREEYD | 103 |
| signaling | VLDKRRGRDPEMGGKPRRKNPQEGLYNELQKD | |
| | KMAEAYSEIGMKGERRRGKGHDGLYQGLSTAT | |
| | KDTYDALHMQALPPR | |
| BFP | MSELIKENMHMKLYMEGTVDNHHFKCTSEGEG | 104 |
| | KPYEGTQTMRIKVVEGGPLPFAFDILATSFLYGS | |
| | KTFINHTQGIPDFFKQSFPEGFTWERVTTYEDGG | |
| | VLTATQDTSLQDGCLIYNVKIRGVNFTSNGPVM | |
| | QKKTLGWEAFTETLYPADGGLEGRNDMALKLV | |
| | GGSHLIANIKTTYRSKKPAKNLKMPGVYYVDYR | |
| | LERIKEANNETYVEQHEVAVARYCDLPSKLGHK | |
| | LN | |
| P2A | GSGATNFSLLKQAGDVEENPGP | 105 |
| 2173 anti-EGFRvIII scFv | MALPVTALLLPLALLLHAARPEIQLVQSGAEVKK | 140 |
| | PGESLRISCKGSGFNIEDYYIHWVRQMPGKGLEW | |
| | MGRIDPENDETKYGPIFQGHVTISADTSINTVYLQ | |
| | WSSLKASDTAMYYCAFRGGVYWGQGTTVTVSS | |
| | GGGGSGGGGSGGGGSGGGGSDVVMTQSPDSLA | |
| | VSLGERATINCKSSQSLLDSDGKTYLNWLQQKP | |
| | GQPPKRLISLVSKLDSGVPDRFSGSGSGTDFTLTIS | |
| | SLQAEDVAVYYCWQGTHFPGTFGGGTKVEIKTT | |
| | TPAPRPPTPAPTIASQPLSLRPEACRPAAGGAVHT | |
| | RGLDFACDIYIWAPLAGTCGVLLLSLVITLYCKR | |
| | GRKKLLYIFKQPFMRPVQTTQEEDGCSCRFPEEE | |
| | EGGCELRVKFSRSADAPAYKQGQNQLYNELNLG | |
| | RREEYDVLDKRRGRDPEMGGKPRRKNPQEGLY | |
| | NELQKDKMAEAYSEIGMKGERRRGKGHDGLYQ | |
| | GLSTATKDTYDALHMQALPPR | |
| P5A2 anti-BCMAscFv | E V Q L L E S G G G L V Q P G G S L R | 141 |
| | L S C A A S G F T F S S Y A M N W V R | |
| | Q A P G K G L E W V S A I S D S G G S | |
| | T Y Y A D S V K G R F T I S R D N S K | |
| | N T L Y L Q M N S L R A E D T A V Y Y | |
| | C A R Y W P M D I W G Q G T L V T V S | |
| | S G G G G S G G G G S G G G G S E I V | |
| | L T Q S P G T L S L S P G E R A T L S | |
| | C R A S Q S V S S S Y L A W Y Q Q K P | |
| | G Q A P R L L M Y D A S I R A T G I P | |
| | D R F S G S G S G T D F T L T I S R L | |
| | E P E D F A V Y Y C Q Q Y G S W P L T | |
| | F G Q G T K V E I K | |
| P5A2 anti-BCMACAR | (M A L P V T A L L L P L A L L L H A A | 142 |
| | R P)E V Q L L E S G G G L V Q P G G S L | |
| | R L S C A A S G F T F S S Y A M N W V R | |
| | Q A P G K G L E W V S A I S D S G G S T | |
| | Y Y A D S V K G R F T I S R D N S K N T | |
| | L Y L Q M N S L R A E D T A V Y Y C A R | |
| | Y W P M D I W G Q G T L V T V S S G G G | |
| | G S G G G G S G G G G S E I V L T Q S P | |
| | G T L S L S P G E R A T L S C R A S Q S | |
| | V S S S Y L A W Y Q Q K P G Q A P R L L | |
| | M Y D A S I R A T G I P D R F S G S G S | |
| | G T D F T L T I S R L E P E D F A V Y Y | |
| | C Q Q Y G S W P L T F G Q G T K V E I K | |
| | G S G G G G S C P Y S N P S L C S G G G | |
| | G S C P Y S N P S L C S G G G G S T T T | |
| | P A P R P P T P A P T I A S Q P L S L R | |
| | P E A C R P A A G G A V H T R G L D F A | |
| | C D I Y I W A P L A G T C G V L L L S L | |
| | V I T L Y C K R G R K K L L Y I F K Q P | |
| | F M R P V Q T T Q E E D G C S C R F P E | |
| | E E E G G C E L R V K F S R S A D A P A | |
| | Y Q Q G Q N Q L Y N E L N L G R R E E Y | |
| | D V L D K R R G R D P E M G G K P R R K | |
| | N P Q E G L Y N E L Q K D K M A E A Y S | |
| | E I G M K G E R R R G K G H D G L Y Q G | |
| | L S T A T K D T Y D A L H M Q A L P P R | |

In some embodiments, the CAR-immune cell (e.g., CAR-T cell) of the disclosure comprises a polynucleotide encoding a suicide polypeptide, such as for example RQR8. See, e.g., WO2013153391A, which is hereby incorporated by reference in its entirety. In some embodiments, a suicide polypeptide is expressed on the surface of the cell. In some embodiments, a suicide polypeptide is included in the CAR construct. In some embodiments, a suicide polypeptide is not part of the CAR construct.

In some embodiments, the extracellular domain of any one of CARs disclosed herein may comprise one or more epitopes specific for (specifically recognized by) a monoclonal antibody. These epitopes are also referred to herein as mAb-specific epitopes. Exemplary mAb-specific epitopes are disclosed in International Patent Publication No. WO 2016/120216, which is incorporated herein in its entirety. In these embodiments, the extracellular domain of the CARs comprise antigen binding domains that specifically bind to a target of interest and one or more epitopes that bind to one or more monoclonal antibodies (mAbs). CARs comprising the mAb-specific epitopes can be single-chain or multi-chain.

The inclusion of epitopes specific for monoclonal antibodies in the extracellular domain of the CARs described herein allows sorting and depletion of engineered immune cells expressing the CARs. In some embodiments, allowing for depletion provides a safety switch in case of deleterious effects, e.g., upon administration to a subject.

Methods of preparing engineered immune cells for use in immunotherapy are also provided herein. In some embodiments, the methods comprise introducing a CACCR and a CAR into immune cells, and expanding the cells. In some embodiments, the invention relates to a method of engineering an immune cell comprising: providing a cell and expressing a CACCR, and expressing at the surface of the cell at least one CAR. In some embodiments, the method comprises: transfecting the cell with at least one polynucleotide encoding a CACCR, and at least one polynucleotide encoding a CAR, and expressing the polynucleotides in the cell. In some embodiments, the method comprises: transfecting the cell with at least one polynucleotide encoding a CACCR, at least one polynucleotide encoding a CAR, and expressing the polynucleotides in the cell.

In some embodiments, the polynucleotides encoding the CACCR and CAR are present in one or more expression vectors for stable expression in the cells. In some embodiments, the polynucleotides are present in viral vectors for stable expression in the cells. In some embodiments, the viral vectors may be for example, lentiviral vectors or adenoviral vectors.

In some embodiments, polynucleotides encoding polypeptides according to the present disclosure can be mRNA which is introduced directly into the cells, for example by electroporation. In some embodiments, CytoPulse electroporation technology, such as PulseAgile, can be used to transiently permeabilize living cells for delivery of material into the cells (e.g. U.S. Pat. No. 6,078,490; PCT/US2011/000827; and PCT/US2004/005237). Parameters can be modified in order to determine conditions for high transfection efficiency with minimal mortality.

Also provided herein are methods of transfecting an immune cell, e.g a T-cell. In some embodiments, the method comprises: contacting a T-cell with RNA and applying to the T-cell an agile pulse sequence. In some embodiments, a method of transfecting an immune cell (e.g. T-cell) comprising contacting the immune cell with RNA and applying to the cell an agile pulse sequence.

In some embodiments, the method can further comprise a step of genetically modifying a cell by inactivating at least one gene expressing, for example without limitation, a component of the TCR, a target for an immunosuppressive agent, an HLA gene, and/or an immune checkpoint protein such as, for example, PDCD1 or CTLA-4. By inactivating a gene it is intended that the gene of interest is not expressed in a functional protein form. In some embodiments, the gene to be inactivated is selected from the group consisting of, for example without limitation, TCRα, TCRβ, CD52, GR, deoxycytidine kinase (DCK), PD-1, and CTLA-4. In some embodiments the method comprises inactivating one or more genes by introducing into the cells a rare-cutting endonuclease able to selectively inactivate a gene by selective DNA cleavage. In some embodiments the rare-cutting endonuclease can be, for example, a transcription activator-like effector nuclease (TALE-nuclease) or CRISPR-based endonuclease (e.g Cas-9 or Cas12a).

In another aspect, a step of genetically modifying cells can comprise: modifying immune cells (e.g. T-cells) by inactivating at least one gene expressing a target for an immunosuppressive agent, and; expanding the cells, optionally in presence of the immunosuppressive agent.

In some embodiments, the engineered immune cells (e.g. T-cells) provided herein exhibit improved cytotoxicity, increased expansion, and/or increased levels of memory phenotype markers relative to engineered immune cells that do not express the CACCR.

In some embodiments, the engineered immune cells (e.g. T-cells) provided herein exhibit (i) increased in vivo persistence, (ii) increased STAT activation, (iii) increased cytotoxicity, (iv) increased levels of memory phenotype markers, (v) increased expansion (proliferation), or combinations of these functional features constitutively, relative to engineered immune cells that do not express the CACCR. In some embodiments, the improvement in the one or more functional features described herein tunable, dependent upon the mutations/modifications introduced to the CACCR. In some embodiments, STATs activated by the engineered immune cell comprising one or more CACCRs disclosed are STAT1, STAT2, STAT3, STAT4, STAT5, STAT6, or combinations thereof. In one embodiment, memory phenotype markers increased or maintained by the immune cell comprising the CACCR include stem cell memory (Tscm) marker and central memory (Tcm) marker.

In some embodiments, the improvement in one or more functional features exhibited by an engineered immune cell comprising an CACCR provided herein is at least about 2 fold, 2.5 fold, 3 fold, 3.5 fold, 4 fold, 4.5 fold, 5 fold, 6 fold, 7 fold, 8 fold, 9 fold, fold, 15 fold, 20 fold, 25 fold, 30 fold, 40 fold, 50 fold, 60 fold, 70 fold, 80 fold, 90 fold, 100 fold, 125 fold, 150 fold, 200 fold, 250 fold, 300 fold, 350 fold, 400 fold, 450 fold, or even about 10500 fold, including values and ranges therebetween, compared to an immune cell that does not express the CACCR.

In some embodiments, the improvement in one or more functional features exhibited by an engineered immune cell comprising a CACCR provided herein is at least about 10%, 20%, 25%, 30%, 35%, 40%, 45%, 50%, 55%, 60%, 70%, 75%, 80%, 90%, 100%, 125%, 150%, 200%, 250%, 300%, 350%, 400%, or even about 80% 500%, including values and ranges therebetween, compared to an engineered immune cell that does not express the CACCR.

### III. Therapeutic Methods

Provided herein are pharmaceutical compositions comprising cells bearing the CACCRs and CARs of the disclosure.

Engineered CACCR-bearing and CAR-bearing immune cells (e.g. T-cells) obtained by the methods described above, or cell lines derived from such engineered immune cells, can be used as a medicament. In some embodiments, such a medicament can be used for treating a disorder such as for example a viral disease, a bacterial disease, a cancer, an inflammatory disease, an immune disease, or an aging-associated disease. In some embodiments, the cancer is a solid cancer. In some embodiments the cancer is a liquid cancer. The cancer can be selected from the group consisting of gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma. In some embodiments, the subject is a previously treated adult subject with locally advanced or metastatic melanoma, squamous cell head and neck cancer (SCHNC), ovarian carcinoma, sarcoma, or relapsed or refractory classic Hodgkin's Lymphoma (cHL).

In some embodiments, engineered immune cells, or a cell line derived from the engineered immune cells, can be used in the manufacture of a medicament for treatment of a disorder in a subject in need thereof. In some embodiments, the disorder can be, for example, a cancer, an autoimmune disorder, or an infection.

Also provided herein are methods for treating subjects in need of such treatment.

As used herein, the term “subject” refers to any vertebrate including, without limitation, humans and other primates (e.g., chimpanzees, cynomologous monkeys, and other apes and monkey species), farm animals (e.g., cattle, sheep, pigs, goats and horses), domestic mammals (e.g., dogs and cats), laboratory animals (e.g., rabbits, rodents such as mice, rats, and guinea pigs), and birds (e.g., domestic, wild and game birds such as chickens, turkeys and other gallinaceous birds, ducks, geese, and the like). In some embodiments, the subject is a mammal. In exemplary embodiments, the subject is a human.

In some embodiments the method comprises providing immune cells of the disclosure, bearing the CACCRs and CARs described herein to a subject in need thereof.

In some embodiments, CACCR and CAR-bearing T-cells of the invention can undergo robust in vivo T-cell expansion and can persist for an extended amount of time.

Methods of treatment of the invention can be ameliorating, curative or prophylactic. The method of the invention may be either part of an autologous immunotherapy or part of an allogenic immunotherapy treatment.

In another aspect, the invention provides a method of inhibiting tumor growth or progression in a subject who has a tumor, comprising administering to the subject an effective amount of CACCR-expressing and CAR-expressing immune cells as described herein. In another aspect, the invention provides a method of inhibiting or preventing metastasis of cancer cells in a subject, comprising administering to the subject in need thereof an effective amount of engineered immune cells as described herein. In another aspect, the invention provides a method of inducing tumor regression in a subject who has a tumor, comprising administering to the subject an effective amount of engineered immune cells as described herein.

In some embodiments, the engineered T-cells herein can be administered parenterally in a subject.

Also provided is the use of any of the engineered T-cells provided herein in the manufacture of a medicament for the treatment of cancer or for inhibiting tumor growth or progression in a subject in need thereof.

In some embodiments, treatment can be administrated into subjects undergoing an immunosuppressive treatment. Indeed, the invention preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the invention within the subject. The administration of the cells or population of cells according to the invention may be carried out in any convenient manner, including by aerosol inhalation, injection, ingestion, transfusion, implantation or transplantation. The compositions described herein may be administered to a subject subcutaneously, intradermally, intratumorally, intranodally, intramedullary, intramuscularly, by intravenous or intralymphatic injection, or intraperitoneally. Cells bearing the CACCRs and CARs of the disclosure or the pharmaceutical compositions thereof may be administered via one or more of the following routes of administration: intravenous, intraocular, intravitreal, intramuscular, subcutaneous, topical, oral, transdermal, intraperitoneal, intraorbital, by implantation, by inhalation, intrathecal, intraventricular, via the car, or intranasal.

In some embodiments the administration of the cells or population of cells (bearing the CACCRs and CARs of the disclosure) can comprise administration of, for example, about 10⁴ to about 10⁹ cells per kg body weight including all integer values of cell numbers within those ranges. In some embodiments the administration of the cells or population of cells can comprise administration of about 10⁴ to 10⁵ cells per kg body weight, 10⁵ to 10⁶ cells per kg body weight, 10⁶ to 10⁷ cells per kg body weight, 10⁷ to 10⁸ cells per kg body weight, or 10⁸ to 10⁹ cells per kg body weight. The cells or population of cells can be administrated in one or more doses. In some embodiments, said effective amount of cells can be administrated as a single dose. In some embodiments, said effective amount of cells can be administrated as more than one dose over a period time. Timing of administration is within the judgment of managing physician and depends on the clinical condition of the subject. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In some embodiments, an effective amount of cells or composition comprising those cells are administrated parenterally. In some embodiments, administration can be an intravenous administration. In some embodiments, administration can be directly done by injection within a tumor.

The methods can further comprise administering one or more agents to a subject prior to administering the engineered immune cells bearing a CAR and a CACCR provided herein. In certain embodiments, the agent is a lymphodepleting (preconditioning) regimen. For example, methods of lymphodepleting a subject in need of such therapy comprise administering to the subject specified beneficial doses of cyclophosphamide (between 200 mg/m²/day and 2000 mg/m²/day, about 100 mg/m²/day and about 2000 mg/m²/day; e.g., about 100 mg/m²/day, about 200 mg/m²/day, about 300 mg/m²/day, about 400 mg/m²/day, about 500 mg/m²/day, about 600 mg/m²/day, about 700 mg/m²/day, about 800 mg/m²/day, about 900 mg/m²/day, about 1000 mg/m²/day, about 1500 mg/m²/day or about 2000 mg/m²/day) and specified doses of fludarabine (between 20 mg/m²/day and 900 mg/m²/day, between about 10 mg/m²/day and about 900 mg/m²/day; e.g., about 10 mg/m²/day, about 20 mg/m²/day, about 30 mg/m²/day, about 40 mg/m²/day, about 40 mg/m²/day, about 50 mg/m²/day, about 60 mg/m²/day, about 70 mg/m²/day, about 80 mg/m²/day, about 90 mg/m²/day, about 100 mg/m²/day, about 500 mg/m²/day or about 900 mg/m²/day). An exemplary dosing regimen involves treating a subject comprising administering daily to the patient about 300 mg/m²/day of cyclophosphamide in combination or before or after administering about 30 mg/m²/day of fludarabine for three days prior to administration of a therapeutically effective amount of engineered immune cells to the patient.

In some embodiments, notably in the case when the engineered cells provided herein have been gene edited to eliminate or minimize surface expression of CD52, lymphodepletion further comprises administration of an anti-CD52 antibody, such as alemtuzumab. In some embodiments, the CD52 antibody is administered at a dose of about 1-20 mg/day IV, e.g., about 13 mg/day IV for 1, 2, 3 or more days. The antibody can be administered in combination with, before, or after administration of other elements of a lymphodepletion regime (e.g., cyclophosphamide and/or fludarabine).

In certain embodiments, compositions comprising CACCR and CAR-expressing immune effector cells disclosed herein may be administered in conjunction with any number of chemotherapeutic agents.

### IV. Kits and Articles of Manufacture

The present disclosure provides kits comprising any one or more of the CACCRs and CAR-bearing cells described herein, and pharmaceutical compositions thereof. The present disclosure also provides articles of manufacture comprising any one or more of the CACCRs and CAR-bearing CAR-I cells described herein, pharmaceutical compositions thereof, and kits described herein.

The following examples are included for illustrative purposes and are not intend to limit the scope of the disclosure.

All patent and non-patent documents referenced throughout this disclosure are incorporated by reference herein in their entirety for all purposes

### EXAMPLES

### Example 1: Identification of TpoR TM Mutants that Constitutively Activate Cytokine Signaling

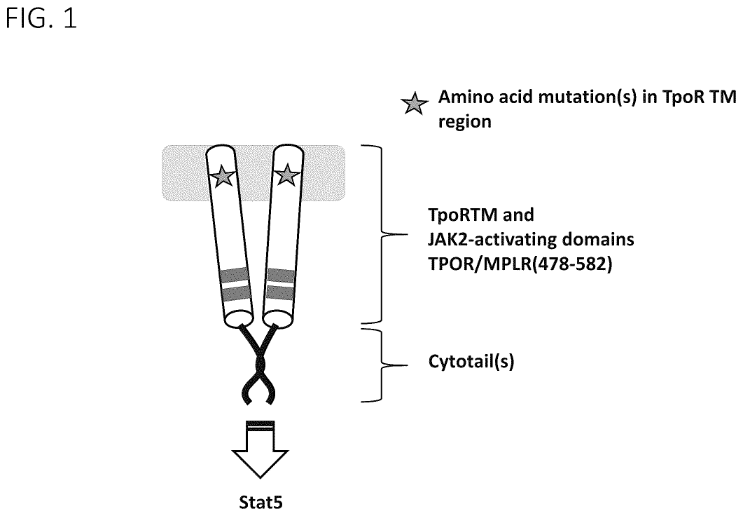

A prototypic constitutively active chimeric cytokine receptor (CACCR) was designed, using sequences from the thrombopoietin receptor (TpoR). TpoR is capable of activating the JAK-Stat signaling pathway and signals as homodimeric receptor. Single point mutations (amino acid substitutions) in TpoR activity have been shown to modulate receptor activity (Proc Natl Acad Sci USA. 2013 Feb. 12; 110(7): 2540-5.; FASEB J. 2011 July; 25(7): 2234-44.; J Biol Chem. 2016 Feb. 5; 291(6): 2974-87). In this example, a constitutively active chimeric cytokine receptor was engineered from a naturally occurring TpoR receptor: the extracellular domain of the natural TpoR receptor was removed, so it no longer has ligand-binding ability; 1-3 mutations were introduced into its transmembrane domain; and the TpoR cytotail was substituted with that of the desired/described cytokine receptor. FIG. 1 shows a schematic of the engineered constitutively active chimeric cytokine receptor.

To demonstrate the utility of the constitutively active chimeric cytokine receptor in the context of CAR-T-cells, each TpoR transmembrane (TM) variant was cloned into a lentiviral vector encoding a second generation EGFRvIII-specific CAR (2173scFv; described in Sci Transl Med. 2015 Feb. 18; 7(275): 275ra22.). To permit stoichiometric co-expression of the cytokine receptor and the CAR, both genes were linked via a P2A peptide. To facilitate the detection of transduced cells, a v5 epitope tag (KPIPNPLLGLDST) SEQ ID NO: 144) was inserted between the scFv and CD8 hinge domain.

FIG. 2 shows a schematic of the lentiviral vector used to co-express the constitutively active chimeric cytokine receptor and the CAR.

Table 4 shows sequences relating to the constructs used.

A HEK293T-cell reporter assay was used to screen for TpoR TM variants capable of constitutive cytokine signaling. Briefly, 20,000 HEK293T-cells were plated into each well of a poly-L-lysine-coated 96-well flat-bottom plate and allowed to adhere overnight. A cytokine receptor-CAR construct (2.5 ng), a Stat response element that drives Firefly Luciferase (100 ng; Promega) and Renilla Luciferase control reporter vector (1 ng; Promega) were mixed in a final volume of 5 uL in Opti-MEM (Gibco) (“DNA mix”). As a negative control, cells were transfected with a BFP CAR construct that lacks all cytokine signaling domains. As a positive control, cells were transfected with a vector encoding full-length human EpoR (an erythropoietin receptor in place of the cytokine receptor-CAR construct) so that Stat5 signaling could be induced by the addition of exogeneous recombinant human Epo. 0.3 uL Lipofectamine 2000 (Invitrogen) in 5 uL Opti-MEM was incubated at room temperature for 5 minutes and then added to the DNA mix. The mixture was incubated at room temperature for 20 minutes and the total volume of 10 uL was added to each well containing HEK-293T. 48 hours after transfection, Stat5 reporter activity was evaluated using the Dual-Glo Luciferase Assay System (Promega). Fold induction of Stat5 reporter activity was normalized to that of HEK293T-cells transfected with all vectors except for the cytokine receptor and that were left untreated.

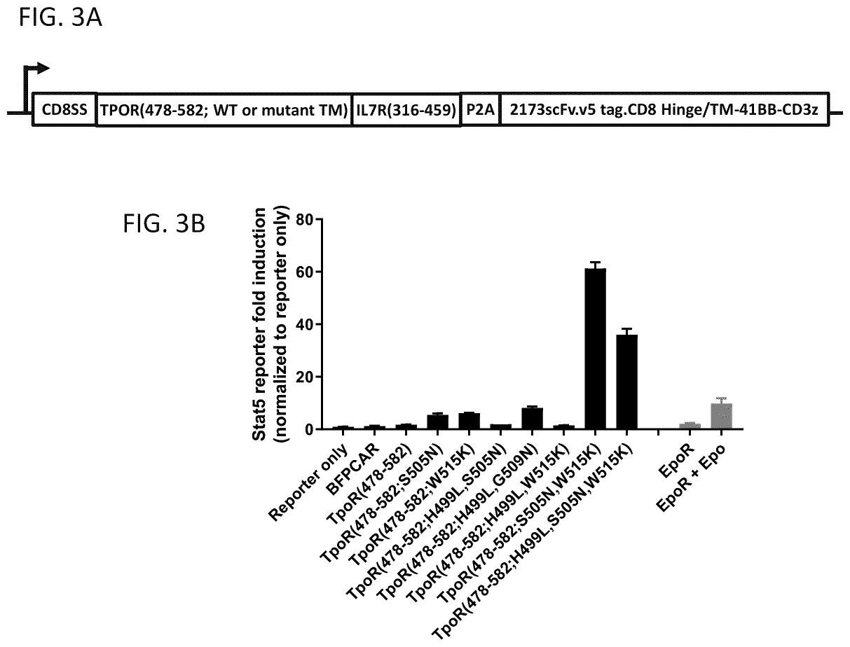

FIGS. 3a and 3b show the identification of TpoR TM mutants that constitutively activate cytokine receptor signaling. FIG. 3a shows a schematic of the lentiviral vector used. It bears the IL7R (316-459) cytotail to mimic IL7 signaling in CAR-T-cells. FIG. 3b shows Stat5 reporter activity as determined by the Dual-Glo luciferase assay. The cytokine receptor bearing the wildtype TpoR TM domain (TpoR(478-582)) did not spontaneously activate Stat5. The TpoR(478-582; S505N), TpoR(478-582; W515K) and TpoR(478-582; H499L,G509N) mutants led to weak Stat5 activation. The TpoR(478-582; H499L,S505N, W515K) permitted a moderate Stat5 activity, while the TpoR(478-582; S505N, W515K) generated the strongest Stat5 signal.

### Example 2: Generation of CAR-T-Cells Expressing Constitutively Active Chimeric Cytokine Receptors

We next tested whether these cytokine receptors signaled in the context of primary human CAR-T-cells. To make lentivirus encoding cytokine receptor-CARs, HEK293T-cells were plated at 0.45 million cells per mL in 2 mL of DMEM (Gibco) supplemented with 10% FBS (Hyclone) per well of a 6-well plate the day before transfection. On the day of transfection, the lentivirus was prepared by mixing together lentiviral packaging vectors 1.5 ug psPAX2, 0.5 ug pMD2G, and 0.5 ug of the appropriate transfer CAR vector in 250 uL Opti-MEM (Gibco) per well of the 6-well plate (“DNA mix”). 10 uL Lipofectamine 2000 (Invitrogen) in 250 uL Opti-MEM was incubated at room temperature for 5 minutes and then added to the DNA mix. The mixture was incubated at room temperature for 20 minutes and the total volume of 500 μL was slowly added to the sides of the wells containing HEK293T. 1 day post-transfection, the media from each well of HEK293T-cells in the 6-well plate was replaced with 2 mL per well of T-cell transduction media, i.e., X-Vivo-15 supplemented with 10% FBS. 2 days post-transfection, The lentiviral supernatants from HEK293T-cells were harvested and passed through a 0.45 micron filter (EMD Millipore) to remove cell debris, concentrated 25-folds using the Lenti-X Concentrator (Takara Bio) according to manufacturer's instructions and flash-frozen in aliquots. Lentiviral titers were determined by thawing an aliquot of the frozen lentivirus, making 4-fold serial dilutions and performing limiting dilution titration on JurkaT-cells (Clone E6-1; ATCC). On Day 0, purified T-cells were activated in X-Vivo-15 medium (Lonza) supplemented with 100 IU/mL human IL-2 (Miltenyi Biotec), 10% FBS (Hyclone), and human T TransAct (Miltenyi Biotec, Cat #130-111-160, 1:100 dilution) in a Grex-24 plate (Wilson Wolf, cat #80192M). On Day2, T-cells were resuspended at 0.5 million cells per mL in T-cell transduction media, transduced with the respective lentiviral stocks at MOI=5 along with 100 IU/mL human IL-2 in a Grex-24 plate. On Day 5 when transduction was complete, cells were harvested and washed to remove residual IL-2. They were then resuspended in T-cell expansion media, i.e., X-Vivo-15 supplemented with 5% human AB serum (Gemini Bio), and each sample was divided equally into 2 parts, with one part receiving 100 IU/mL human IL-2 as per standard protocol, and the other receiving a lower concentration of 25 IU/mL human IL-2. Cells were expanded into larger G-Rex vessels (Wilson Wolf) as needed using T-cell expansion media and the respective concentrations of human IL-2. On Days 5, 9 and 14, the absolute number of T-cells in each sample was counted, and transduction efficiency was determined by detecting the percentage of T-cells that bound a FITC-conjugated v5 tag monoclonal antibody (Thermo Fisher) using flow cytometry. On Day 14 or 15, the CAR-T-cell products were cryopreserved and thawed as needed for further assays.

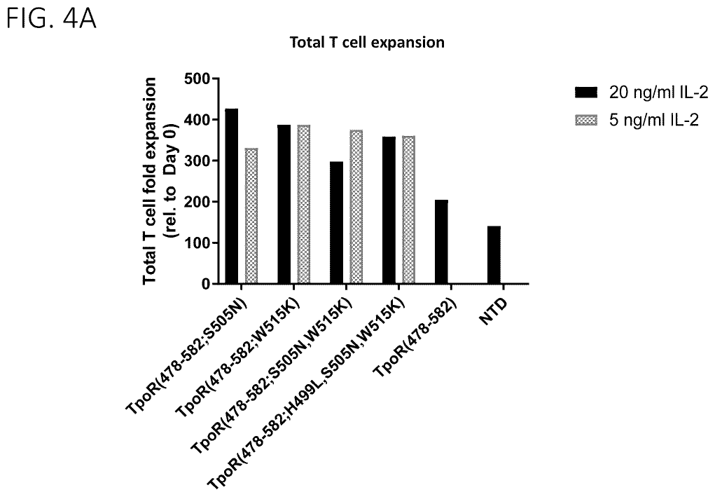

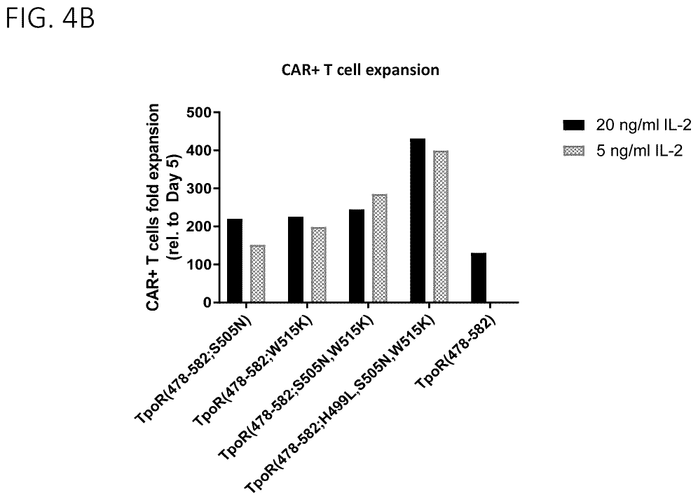

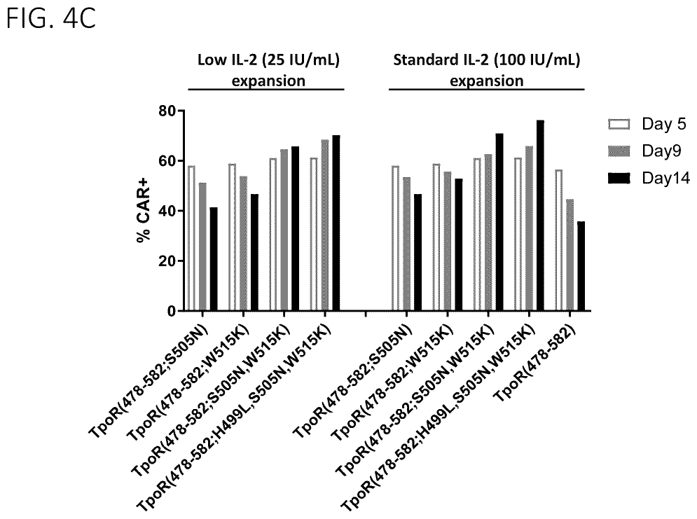

FIGS. 4a-4c show results for the generation of CAR-T-cells coexpressing a constitutively active chimeric cytokine receptor. Compared to CAR-T-cell cultures with the wildtype TpoR TM cytokine receptor (TpoR(478-582)), CAR-T-cell cultures bearing TpoR TM mutants underwent more robust expansion in terms of both total T-cell numbers (FIG. 4a) and CAR-T-cell numbers (FIG. 4b). FIGS. 4a-4c show that transduction efficiencies of TpoR TM mutants was equal or more better than their wildtype TpoR(478-582) counterparts. TpoR TM mutants permit a greater yield of CAR-T-cell product. Furthermore, expanding TpoR TM mutants in lower IL-2 concentrations did not impact CAR-T-cell expansion or yield (FIG. 4c).

On Day 14 of CAR-T-cell production, the memory phenotype of CAR-T-cells were determined. Briefly, samples were washed with PBS, Fc blocked, then stained with the following antibody cocktail diluted in PBS+1% BSA: BUV395-conjugated anti-human CD3, BV510-conjugated anti-human CD8, BV605-conjugated human CD4 and FITC-conjugated v5 tag (for CAR detection), PE/Cy7-conjugated anti-human CD62L (Biolegend) and BV785-conjugated anti-human CD45RO (Biolegend). Finally, samples were washed in PBS and cell pellets were resuspended in 130 uL PBS+1% BSA for FACS analysis.

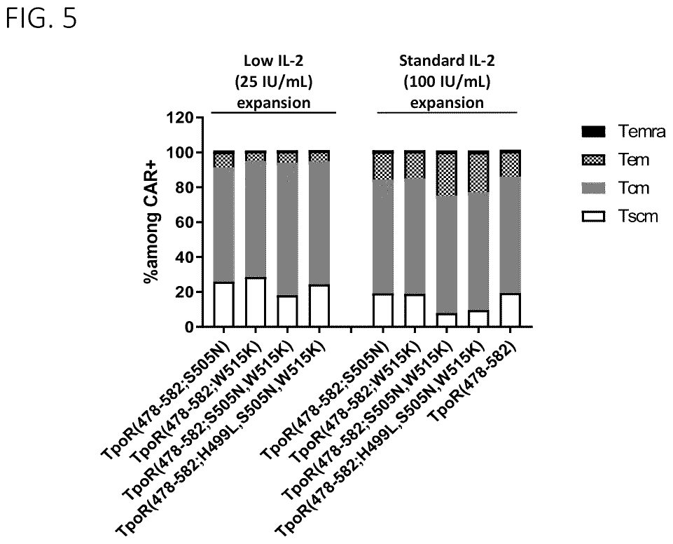

FIG. 5 shows the memory T-cell subset distribution in the CAR-T-cell product. Compared to their wildtype TpoR(478-582) counterpart, TpoR(478-582; W515K) and TpoR(478-582; H499L,G509N) mutants showed greater differentiation when expanded in the standard 100 IU/mL IL-2 conditions. Expansion in low IL-2 conditions ameliorated differentiation. In concert with standard concentrations of IL-2, the stronger Stat5 signaling induced by the TpoR(478-582; W515K) and TpoR(478-582; H499L,G509N) mutants may lead to accelerated CAR-T-cell differentiation, and that expansion in low IL-2 conditions may be more favorable in the context of CAR-T-cell expressing constitutive cytokine receptors.

### Example 3: TpoR TM Mutants Constitutively Activate Cytokine Signaling in Human CAR-T-Cells

To determine strength of cytokine signaling mediated by TpoR TM mutants, CAR-T-cells bearing TpoR TM cytokine receptor variants were serum starved in 100 uL serum-free RPMI (Corning) for 4 hours in humidified incubator at 37° C. with 5% CO₂. As a positive control, exogenous recombinant human IL-7 (10 ng/ml; Miltenyi) was added during the last 30 minutes of the 4-hour serum starvation. After 4 hours, an antibody cocktail comprising BUV395-conjugated anti-human CD3 (Biolegend) and FITC-conjugated v5 tag monoclonal antibody (Thermo Fisher) were added to the cells and allowed to incubate for the final 20 minutes. Cells were then fixed by adding 35 μL of 16% paraformaldehyde to each 100 uL sample and allowed to incubate for 15 minutes at 37° C. Cells were then washed three times with PBS, and permeabilized in 100% cold methanol for 1 or 2 nights at −20° C. On the day of FACS analysis, cells were washed three times with PBS, Fc-blocked, and stained with AlexaFluor647-conjugated anti-mouse/human Stat5 (pY694) (BD Biosciences) diluted in PBS+1% BSA. After a 1 hour incubation at room temperature in the dark, cells were washed three times before FACS analysis.

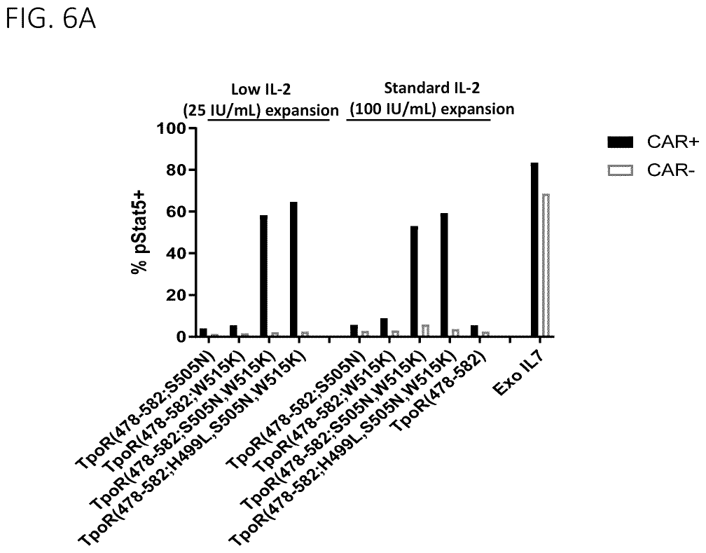

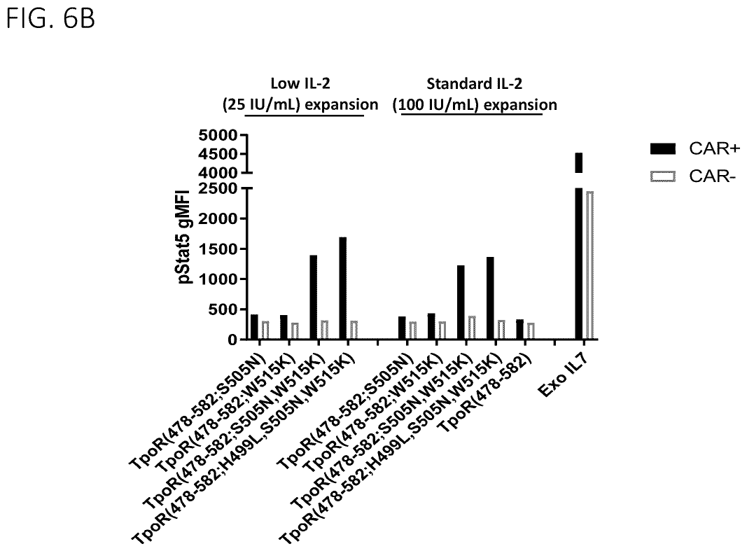

FIGS. 6a-6b show the extent of constitutive cytokine signaling mediated by each TpoR TM variant, as reflected by the percentage of pStat5+ cells (FIG. 6a) and geometric mean fluorescence intensity (gMFI) of pStat5 staining (FIG. 6b). While the TpoR TM single mutants (TpoR(478-582; S505N) and TpoR(478-582; W515K)) did not induce appreciable Stat5 activation, the TpoR TM double mutant (TpoR(478-582; S505N, W515K) and triple mutant (TpoR(478-582; H499L,S505N,W515K)) induced comparably strong constitutive Stat5 activation. CAR-T-cells that were expanded in low IL-2 and standard IL-2 concentrations generated comparable Stat activation profiles. As Stat5 was activated only CAR bearing T-cells (CAR+), and T-cells not bearing a CAR (CAR-) in the same culture, demonstrating that cytokine signaling was CAR-T-cell-specific.

### Example 4: Constitutive Cytokine Receptor Enhanced CAR-T-Cell Cytotoxic Potency and Prolonged Durability of Response

To test whether constitutive cytokine receptor signaling enhanced the cytotoxic activity of CAR-T-cells, we used U87KO-EGFRvIII-nucGFP as target cells. U87KO-EGFRvIII is a kind gift from Cellectis SA (Paris, France). U87KO-EGFRvIII was derived from the parental cell line, U87MG (ATCC), by first knocking out endogenous wildtype EGFR using Transcription Activator-Like Effector Nucleases (TALEN), and then stably overexpressing full-length human EGFRvIII via lentiviral transduction. To facilitate targe T-cell imaging via the IncuCyte Live Cell Analysis Imaging System, U87KO-EGFRvIII-nucGFP target cells were derived from U87KO-EGFRvIII by a second lentiviral transduction with IncuCyte NucLight Green Lentivirus Reagent (Sartorius). 5,000 U87KO-EGFRvIII-nucGFP target cells were seeded and allowed to attach in 96-well plates with black walls and flat clear bottom in 50 uL RPMI containing 10% FBS (Hyclone), non-essential amino acids, sodium pyruvate and 20-25 mM HEPES. EGFRvIII CAR (2173 scFv) T-cells bearing TpoR TM variant cytokine receptors were thawed and added to plated target cells at Effector: Target (E: T) ratios of 1:8 and 1:2. For comparison, wildtype TpoR(478-582) CAR-T-cells with and without the addition of exogenous recombinant human IL-7 were included in the assay. Where applicable, CAR-T-cells were rechallenged at the indicated timepoint by transferring suspension cells from the original plate to a fresh plate of target cells. Duplicate wells were set up for each condition. Cytotoxicity was determined by enumerating the number of live target cells at each timepoint using the IncuCyte Live Cell Analysis Imaging System.

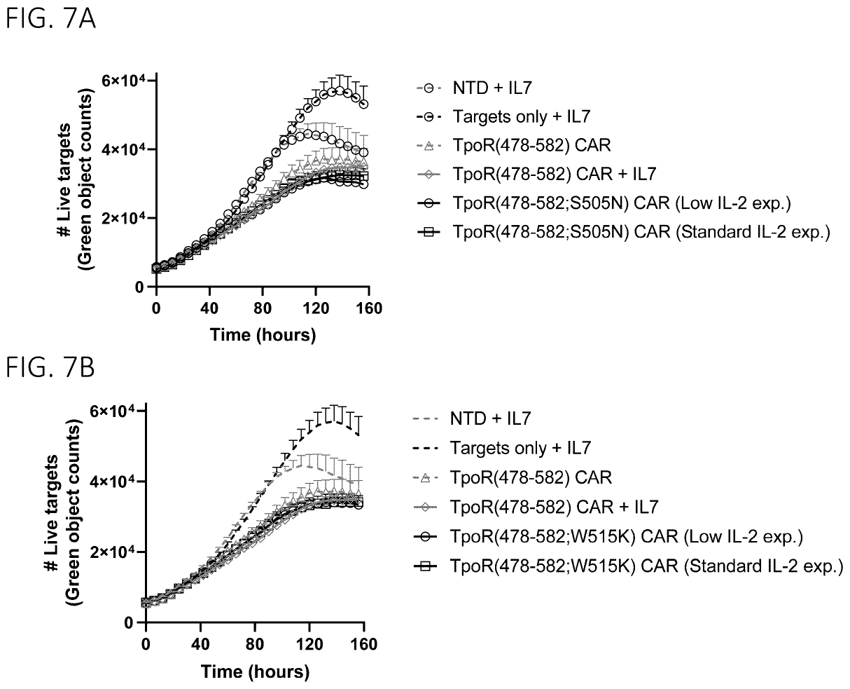

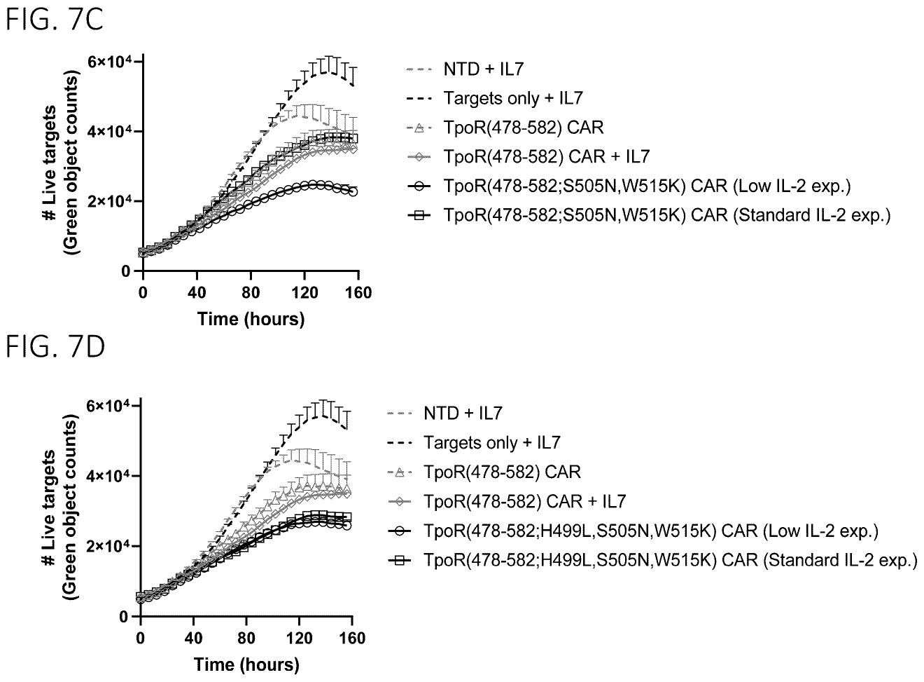

FIGS. 7a-7d shows the cytotoxic activity of TpoR TM mutants at an E:T ratio of 1:8. Consistent with their inability to effectively activate Stat5 (FIGS. 6a-6b), single TpoR TM mutants TpoR(478-582; S505N) (FIG. 7a) and TpoR(478-582; W515KN) (FIG. 7b) did not display functional enhancements compared to their counterparts bearing the wildtype TpoR(478-582) control. FIG. 7c shows that TpoR double mutant CAR-T-cells expanded in standard IL-2 concentrations were not enhanced; whereas TpoR double mutant CAR-T-cells expanded in low IL-2 conditions were more potent at target cell lysis. FIG. 7d shows that TpoR triple mutant CAR-T-cells were more potent at target cell lysis, regardless of the IL-2 concentration during CAR-T-cell production. This indicates that constitutive cytokine receptor signaling enhances CAR-T-cell potency.

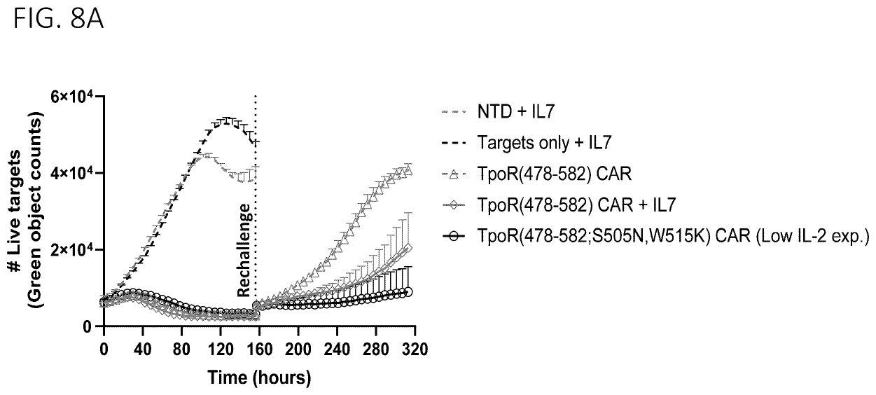

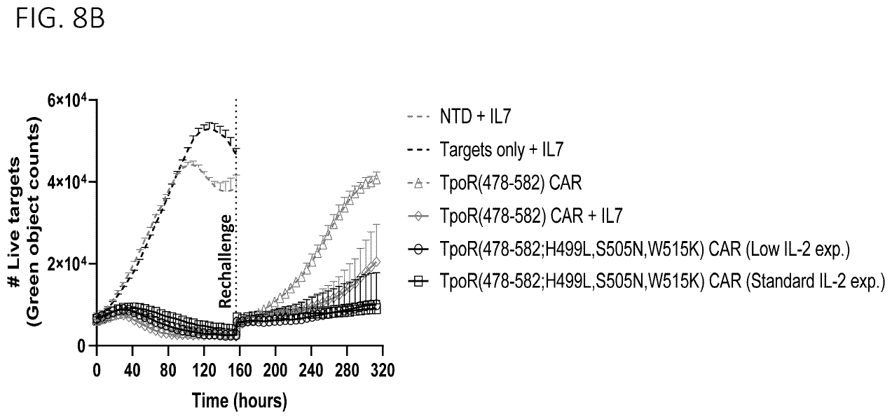

FIGS. 8a-8b show the cytotoxic activity of TpoR TM double (FIG. 8a) and triple mutants (FIG. 8b) at an E:T ratio of 1:2. During the primary response, CAR-T-cells eliminated target ells equally effectively regardless of cytokine receptor activity. However, when re-challenged with fresh targets, only CAR-T-cells expressing constitutively active chimeric cytokine receptors remained functional, indicating that constitutive cytokine receptor signaling enhances the durability of CAR-T-cell responses.

### Example 5: Constitutive Cytokine Receptor Enhanced CAR-T-Cell Persistence and Promoted CAR+ Tscm Expansion

To see the enhancing effects of constitutive cytokine receptor signaling on CAR-T-cell persistence in the absence of targets or exogenous cytokines, a growth factor-independent assay was performed. Briefly, the percentage of CAR-T-cells across all samples were normalized to the sample with the lowest transduction efficiency (35.7%) by the addition of non-transduced (NTD) T-cells. 0.25×10⁶ CAR bearing T-cells cells/mL in 4 mL RPMI containing 10% FBS (Hyclone), non-essential amino acids, sodium pyruvate and 20-25 mM HEPES. Cells were then seeded in T25 tissue culture flasks. As positive controls, exogenous human IL-7 (10 ng/ml; Miltenyi) were added to CAR-T-cells that lacked constitutive cytokine receptor signaling (the wildtype TpoR(478-582).) On the indicated days, duplicate samples of 200 uL were harvested from each condition, and stained using the Zombie NIR Fixable Viability Kit (Biolegend). Samples were washed with PBS, Fc-blocked, then stained with the following antibody cocktail diluted in PBS+1% BSA: BUV395-conjugated anti-human CD3, BV510-conjugated anti-human CD8, BV605-conjugated human CD4 and FITC-conjugated v5 tag (for CAR detection), PE/Cy7-conjugated anti-human CD62L (Biolegend) and BV785-conjugated anti-human CD45RO (Biolegend). Finally, samples were washed in PBS and cell pellets were resuspended in 130 uL PBS+1% BSA containing 123count eBeads counting beads (Thermo Fisher) (10 uL counting beads in 120 uL PBS+1% BSA) prior to FACS analysis.

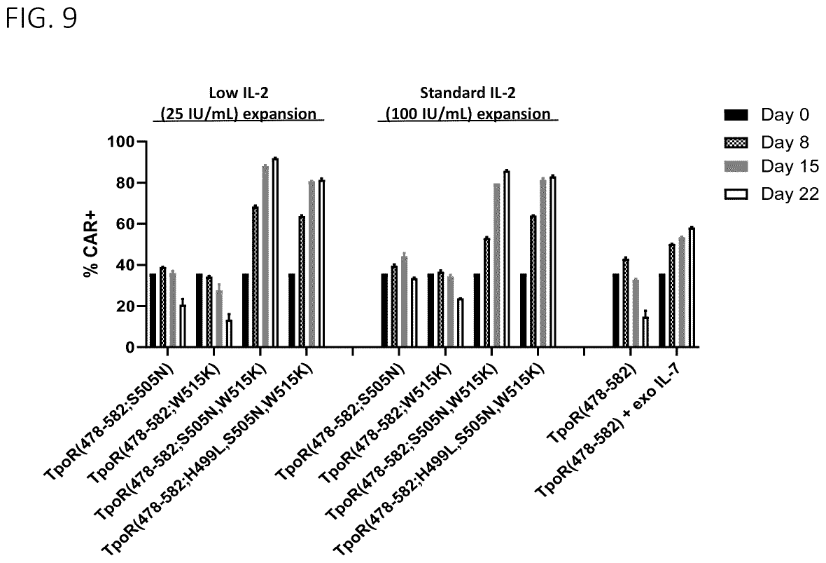

FIG. 9 shows the enrichment of CAR-T-cells over time in the growth factor-independent assay. While CAR-T-cells bearing the wildtype TpoR TM (TpoR(478-582) and TpoR TM single mutants (TpoR(478-582; S505N) and TpoR(478-582; W515KN)) did not enrich, CAR-T-cells bearing the TpoR TM double mutant and triple mutants enriched over time, indicating that CAR bearing T-cells that received constitutive cytokine receptor signaling preferentially survived.

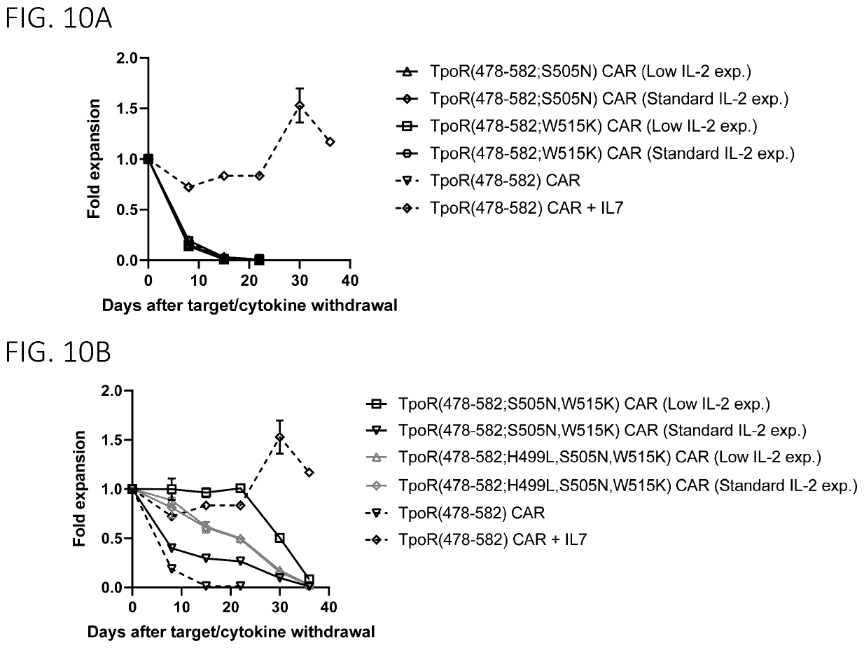

FIGS. 10a-10b shows the fold expansion of CAR-T-cells over time in the growth factor-independent assay. Fold expansion was determined by normalizing the absolute number of CAR-T-cells at each timepoint to the number of CAR-T-cells on Day 0 of the assay. FIG. 10a shows that TpoR TM single mutants that were unable to productively activate cytokine receptor signaling declined at the same rate as CAR-T-cells bearing the wildtype TpoR TM (TpoR(478-582). In contrast, FIG. 10b shows that CAR-T-cells bearing the TpoR TM double mutant or the triple mutant had prolonged survival in the absence of targets and exogenous cytokines. TpoR TM double mutant CAR-T-cells expanded in low IL-2 conditions showed increased persistence, compared to their counterparts that were expanded in standard IL-2 conditions. TpoR TM triple mutant CAR-T-cells manufactured in low and standard IL-2 conditions showed comparable, intermediate enhancement in persistence. Notably, although TpoR double and triple mutant CAR-T-cells persisted longer, they eventually declined, indicating that constitutive cytokine receptor signaling unlikely resulted in CAR-T-cell immortalization or transformation.

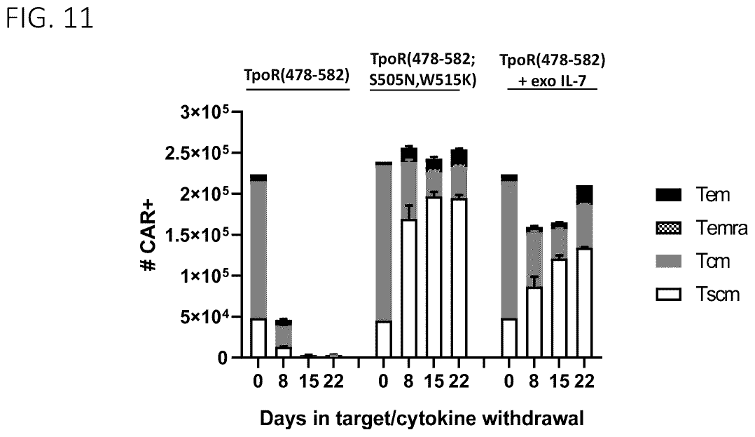

FIG. 11 shows the memory T-cell subset distribution among CAR+ T-cells over time in the growth factor-independent assay. Compared to the wildtype TpoR TM (TpoR(478-582), CAR-T-cells bearing a constitutively-active cytokine receptor (shown in this case is the TpoR TM triple mutant expanded in low IL-2 conditions) showed an expansion in the absolute numbers of stem cell memory T-cell (Tscm), which is the subset that mediates long-lived anti-tumor immunity. Notably, constitutive signaling from the TpoR TM triple mutant was more effective than exogenous human IL-7 supplementation at expanding Tscm CAR-T-cells.

### Example 6: Constitutive Cytotails can be Tailored to Activate Signaling Pathways of Interest

The ability of cytokines to regulate CAR-T cell fate and function stems from their ability to elicit different downstream signaling pathways. For instance, IL-7/IL-2/IL-15-mediated STAT5 activation enhances T cell survival and expansion, whereas IL-12-mediated STAT4 activation drives terminal differentiation to short-lived effectors. Designing recruiting domains (i.e., cytotail) that can mimic a broader range of cytokine signals would offer the flexibility for user-programmable signaling outcomes, thereby conferring control over CAR-T cell fate and function.

To interrogate if CACCRs can transmit signals mediated through additional cytokine receptors, the IL7Ra(316-459) cytotail from FIG. 3 was substituted with alternative cytotails derived from the intracellular signaling domains of IL2Rb or IL12Rb2. A HEK293T cell reporter assay was then used to evaluate the signaling capacity of these chimeras. Briefly, 20,000 HEK293T cells were plated into each well of a poly-L-lysine-coated 96-well flat-bottom plate and allowed to adhere overnight. A cytokine receptor-CAR construct (2.5 ng), a Stat response element that drives Firefly Luciferase (100 ng; Promega) and Renilla Luciferase control reporter vector (1 ng; Promega) were mixed in a final volume of 5 uL in Opti-MEM (Gibco) (“DNA mix”). As a negative control, cells were transfected with either a BFP-CAR construct that lacks all cytokine signaling domains, or a TpoR(478-582). IL7Ra(316-459) construct that lacks the transmembrane mutations and therefore cannot constitutively signal. 0.3 uL Lipofectamine 2000 (Invitrogen) in 5 uL Opti-MEM was incubated at room temperature for 5 minutes and then added to the DNA mix. The mixture was incubated at room temperature for 20 minutes and the total volume of 10 uL was added to each well containing HEK-293T. 48 hours after transfection, activity of the respective Stat reporters was evaluated using the Dual-Glo Luciferase Assay System (Promega). Fold induction of Stat5 reporter activity was normalized to that of HEK293T cells transfected with the control BFP-CAR construct.

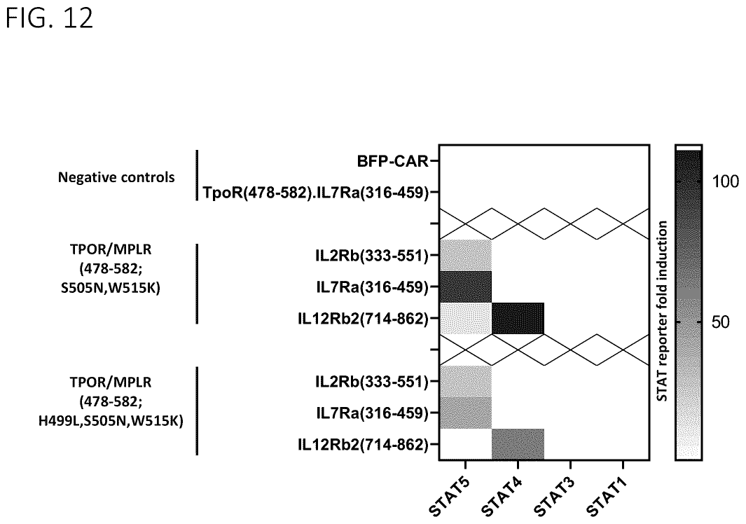

FIG. 12 shows that construct bearing different signaling domains preferentially activate different STAT pathways. Specifically, IL2Rb(333-551) and IL7Ra(316-459) activated STAT5, while IL12Rb2(714-862) activated STAT4, mirroring signaling expected of the respective parental receptors. This demonstrates that CACCRs can be programmed to activate desired signaling pathways by fusion with the signaling domain of interest. Furthermore, consistent with FIG. 3, CACCRs bearing the TpoR(478-582; S505N, W515K) dimerization domain effected stronger signaling than their TpoR(478-582; H499L,S505N, W515K) counterpart. The strength of CACCR signaling outputs can therefore be further tuned by fusion with either of these dimerization domains.

### Example 7: CACCR Signaling Domain can be Optimized to Modulate Signal Strength while Reducing Vector Cargo Size

Currently, viral-based gene delivery methods (e.g. lentiviral- and retroviral-mediated gene transfers) are routinely used for CAR-T cell manufacturing. As cargo size increases, transduction efficiency and CAR-T cell yield decreases. Reducing the size of the cargo would therefore be beneficial to ensure manufacturing success. The recruiting/signaling domain of CACCR optimization offers a means to this end for two reasons. First, as in the case of the IL2Rb(333-551), cytokine receptor-derived signaling domains can reach over 200 amino acids in length and represent over 650 basepairs in the transfer vector. Secondly, while tyrosine residues within signaling domains are important for initiating and propagating downstream signal transduction, some of them can also participate in negative feedback loops that limit signaling duration and strength. Trimming the cytotail signaling domain therefore not only allows vector cargo to be reduced in size, but also provides the opportunity for cytotail signaling to be modulated. To this end, we identified tyrosine residues within the full-length IL12Rb2(714-862) and IL2Rb(331-551) tails and generated variants to identify truncated constructs capable of mediating cytotail signaling.

The full-length IL12Rb2(714-862) contains two phosphorylable tyrosine residues, Y767 and Y800, that may participate in downstream signaling. We generated a truncated IL12Rb2(775-825) tail containing only Y800, and evaluated its ability to activate STAT4 using a HEK293T cell reporter assay. Briefly, 20,000 HEK293T cells were plated into each well of a poly-L-lysine-coated 96-well flat-bottom plate and allowed to adhere overnight. A CACCR-CAR construct (2.5 ng), a Stat response element that drives Firefly Luciferase (100 ng; Promega) and Renilla Luciferase control reporter vector (1 ng; Promega) were mixed in a final volume of 5 uL in Opti-MEM (Gibco) (“DNA mix”). As a negative control, cells were transfected with a BFP-CAR construct that lacks all cytokine signaling domains. 0.3 uL Lipofectamine 2000 (Invitrogen) in 5 uL Opti-MEM was incubated at room temperature for 5 minutes and then added to the DNA mix. The mixture was incubated at room temperature for 20 minutes and the total volume of 10 uL was added to each well containing HEK-293T. 48 hours after transfection, activity of the Stat4 reporter was evaluated using the Dual-Glo Luciferase Assay System (Promega). Fold induction of Stat4 reporter activity was normalized to that of HEK293T cells transfected with the control BFP-CAR construct.

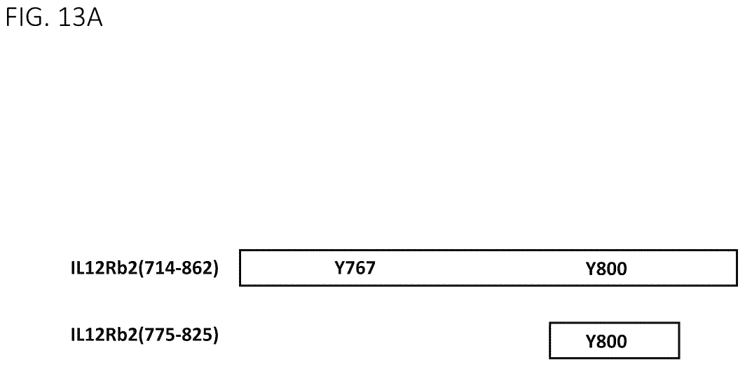

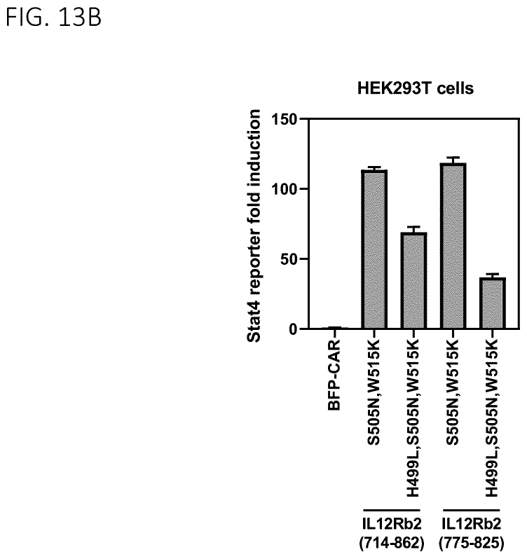

FIGS. 13A-B show the identification of a truncated IL12Rb2(775-825) cytotail capable of STAT4 activation comparable to the full-length IL12Rb2(714-862) cytotail. FIG. 13A shows a schematic diagram of the full-length IL12Rb(714-862) tail and truncated IL12Rb(775-825) cytotail. The positions of tyrosine residues (Y) included in each tail are as indicated. FIG. 13B shows that when fused to the stronger TpoR(478-582; S505N, W515K) dimerization domain, the truncated IL12Rb2(775-825) cytotail fully recapitulated the STAT4 signaling strength of the full-length IL12Rb2(714-862) cytotail. When fused to the weaker TpoR(478-582; H499L,S505N, W515K) dimerization domain, the truncated IL12Rb2(775-825) cytotail partially recapitulated the STAT4 signaling strength of the full-length IL12Rb2(714-862) cytotail.

The full-length IL2Rb(333-551) cytotail contains six tyrosine residues that may participate in downstream signaling. Of these, Y364 (the tyrosine residue closest to the transmembrane domain of the receptor) has been reported to activate PI3K to promote T cell differentiation and proliferation, as well as cytoskeletal reorganization to induce receptor internalization; therefore, while Y364 can promote T cell effector functions, it can also limit IL2Rb signaling strength and duration. We generated truncated IL2Rb cytotails containing three (Y364, Y418 and Y436) out of six tyrosine residues, or two (Y418 and Y436) out of six tyrosine residues, and evaluated their capacity to activate STAT5 using a HEK293T cell reporter assay. Briefly, 20,000 HEK293T cells were plated into each well of a poly-L-lysine-coated 96-well flat-bottom plate and allowed to adhere overnight. A CACCR-CAR construct (2.5 ng), a Stat response element that drives Firefly Luciferase (100 ng; Promega) and Renilla Luciferase control reporter vector (1 ng; Promega) were mixed in a final volume of 5 uL in Opti-MEM (Gibco) (“DNA mix”). As a negative control, cells were transfected with a BFP-CAR construct that lacks all cytokine signaling domains. 0.3 uL Lipofectamine 2000 (Invitrogen) in 5 uL Opti-MEM was incubated at room temperature for 5 minutes and then added to the DNA mix. The mixture was incubated at room temperature for 20 minutes and the total volume of 10 uL was added to each well containing HEK-293T. 48 hours after transfection, activity of the Stat5 reporter was evaluated using the Dual-Glo Luciferase Assay System (Promega). Fold induction of Stat5 reporter activity was normalized to that of HEK293T cells transfected with the control BFP-CAR construct.

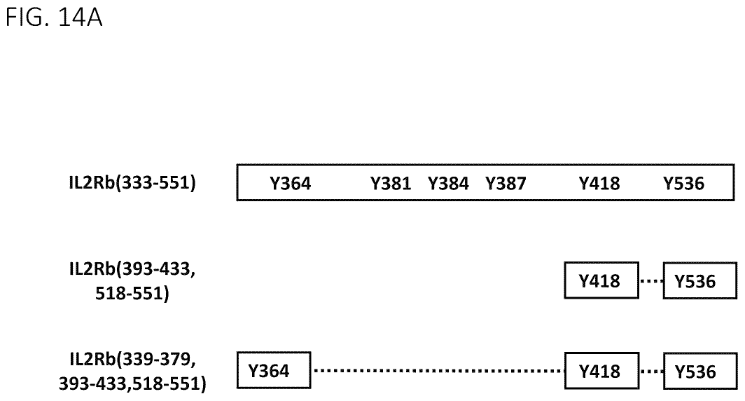

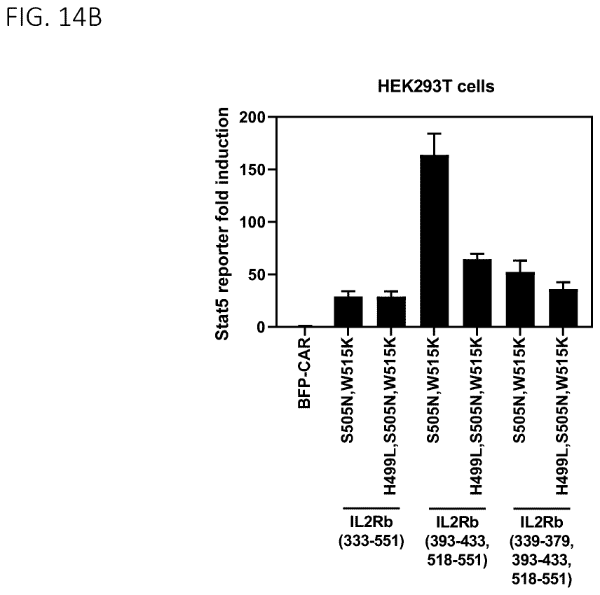

FIGS. 14A-B shows identification of truncated IL2Rb tails capable of equal or better STAT5 activation relative to the full-length IL2Rb(333-551) tail. FIG. 14A shows a schematic of the full-length IL2Rb(333-551) tail and two truncated IL2Rb tails. The positions of tyrosine residues (Y) included in each tail are as indicated. Dotted lines represent interjoining regions in the full-length IL2Rb(333-551) tail that have been removed from the truncated tails. FIG. 14B shows results from a HEK293T cell reporter assay, in which STAT5 signaling of the full-length IL2Rb(333-551) was fully recapitulated by the TpoR(478-582; H499L,S505N, W515K) IL2Rb(339-379,393-433,518-551) cytotail that contained Y364, Y418 and Y536. In the TpoR(478-582; S505N, W515K). IL2Rb(393-433,518-551) cytotail, the additional removal of Y364 that mediates receptor internalization resulted in dramatically improved STAT5 signaling strength.

The HEK293T cell assay is a short-term assay whose readout is measured within 48 hours of cytotail transfection. While it provides an efficient screening platform for cytotail activity, the limited duration of this assay does not reflect the complexities of negative feedback loops that are triggered following long-term constitutive cytokine and cytotail signaling. To more accurately evaluate the long-term signaling activity of reduced IL2Rb tail variants, we generated CACCR CAR-T cells using a 2-week production process and assessed STAT5 activation by intracellular flow cytometry. To this end, CACCR CAR-T cells were serum starved in 100 uL serum-free RPMI (Corning) for 4 hours in humidified incubator at 37° C. with 5% CO₂. As a positive control, exogenous recombinant human IL-2 (10 ng/ml; Miltenyi) was added during the last 30 minutes of the 4-hour serum starvation. After 4 hours, an antibody cocktail comprising BUV395-conjugated anti-human CD3 (Biolegend) and FITC-conjugated v5 tag monoclonal antibody (Thermo Fisher) were added to the cells and allowed to incubate for the final 20 minutes. Cells were then fixed by adding 35 μL of 16% paraformaldehyde to each 100 uL sample and allowed to incubate for 15 minutes at 37° C. Cells were then washed three times with PBS, and permeabilized in 100% cold methanol for 1 or 2 nights at −20° C. On the day of FACS analysis, cells were washed three times with PBS, Fc-blocked, and stained with AlexaFluor647-conjugated anti-mouse/human Stat5 (pY694) (BD Biosciences) diluted in PBS+1% BSA. After a 1 hour incubation at room temperature in the dark, cells were washed three times before FACS analysis.

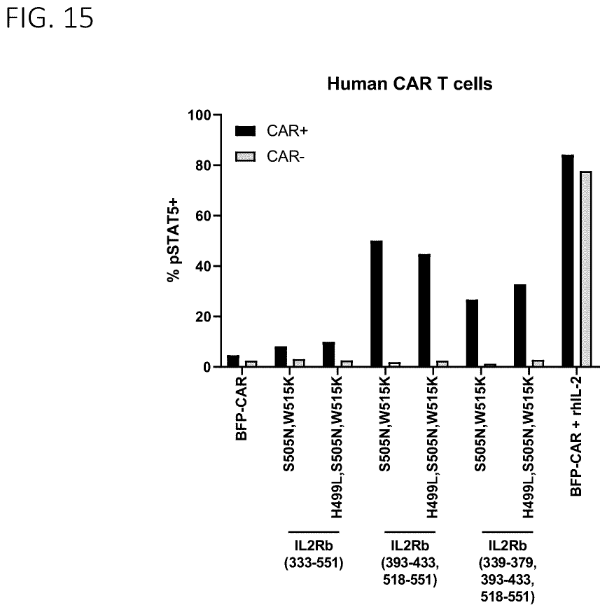

FIG. 15 shows STAT5 activation in primary CACCR CAR-T cells bearing the full-length or truncated IL2Rb cytotails. The greatest STAT5 activation was elicited by CACCR CAR-T cells bearing the truncated IL2Rb(393-433,518-551) cytotail that lacked the Y364 internalization motif. Intermediate STAT5 activation was observed in CACCR CAR-T cells bearing the truncated IL2Rb(339-379,393-433,518-551) cytotail. No STAT5 activation was observed in CAR-negative populations within the same culture, demonstrating the CAR-T cell-specific nature of cytotail signaling. Notably, little to no STAT5 activity was detected in CACCR CAR-T cells bearing the full-length IL2Rb(333-551) cytotail; this may be due to the three other tyrosine residues present in the full-length IL2Rb(333-551) cytotail that may induce long-term, negative regulation. Optimization of cytotail signaling domains to eliminate such negative regulatory motifs is therefore beneficial to ensure that constitutive and productive signaling is maintained in the long-term. As strong cytotails may elicit strong negative feedback response, a weak cytotail may be preferred for long-term stimulation.

### Example 7: Optimized IL2Rb-Derived Cytotails More Closely Mimic Signaling of IL-15, Rather than IL-2

IL-2 and IL-15 are two cytokines that naturally signal through a heterodimeric cytokine receptor comprised of the common-gamma chain and IL2Rb. In spite of sharing the same native receptors, IL-2 and IL-15 exert different effects on T cell differentiation and persistence. Whereas IL-2 induces short-lived effector differentiation, IL-15 promotes the generation of long-lived memory T cells. Furthermore, increased serum concentrations of IL-15 has been shown to correlate positively with patient response to CAR-T cell therapy. Cytotails that mimic the signaling and effects of IL-15, rather than IL-2, are therefore preferred. We sought to determine if the reduced IL2Rb cytotails more closely mimicked IL-2 or IL-15 signaling.

To this end, we utilized CAR-T cells comprising an exemplary CAR bearing the P5A2 scFv directed towards BCMA, coupled to rituximab mimotopes, 4-1BB and CD32 signaling domains (see U.S. Pat. No. 10,294,304, incorporated herein by reference). BCMA specific CAR-T cells co-expressing the truncated IL2Rb tails were generated, and their gene expression profiles compared to control CAR-T cells that had been exposed to exogenous recombinant human IL-2 or IL-15. To make lentivirus encoding CACCR and CARs, HEK293T cells were plated at 0.45 million cells per mL in 2 mL of DMEM (Gibco) supplemented with 10% FBS (Hyclone) per well of a 6-well plate the day before transfection. On the day of transfection, the lentivirus was prepared by mixing together lentiviral packaging vectors 1.5 ug psPAX2, 0.5 ug pMD2G, and 0.5 ug of the appropriate transfer CAR vector in 250 uL Opti-MEM (Gibco) per well of the 6-well plate (“DNA mix”). 10 uL Lipofectamine 2000 (Invitrogen) in 250 uL Opti-MEM was incubated at room temperature for 5 minutes and then added to the DNA mix. The mixture was incubated at room temperature for 20 minutes and the total volume of 500 μL was slowly added to the sides of the wells containing HEK293T. One day post-transfection, the media from each well of HEK293T cells in the 6-well plate was replaced with 2 mL per well of T cell transduction media, i.e., X-Vivo-15 supplemented with 10% FBS. Two days post-transfection, the lentiviral supernatants from HEK293T cells were harvested and passed through a 0.45 micron filter (EMD Millipore) to remove cell debris, and crude lentiviral supernatants were used directly for T cell transduction. On Day 0, purified T cells were activated in X-Vivo-15 medium (Lonza) supplemented with 100 IU/mL human IL-2 (Miltenyi Biotec), 10% FBS (Hyclone), and human T TransAct (Miltenyi Biotec, Cat #130-111-160, 1:100 dilution) in a Grex-24 plate (Wilson Wolf, cat #80192M). On Day2, T cells were resuspended at 0.5 million cells per mL in T cell transduction media, transduced with an equal volume of crude lentiviral supernatant along with 100 IU/mL human IL-2 in a Grex-24 plate. On Day 5, cytotail expressing CAR-T cells were fed by replacing the spent media with T cell expansion media, i.e., X-Vivo-15 supplemented with 5% human AB serum (Gemini Bio), along with 100 IU/mL human IL-2. At this time, control CAR-T cells lacking cytotails were expanded in either 100 U/mL human IL-2 only, or 100 U/mL human IL-2 and 10 ng/ml human IL-15 (Miltenyi Biotec). Cells were expanded into larger G-Rex vessels (Wilson Wolf) as needed using T cell expansion media and the respective concentrations of recombinant cytokines. On Day 13, cells were stained with the Zombie NIR Fixable Viability Kit (Biolegend), labelled with a BUV395-conjugated CD3 antibody (Biolegend) and an anti-idiotype antibody specific for the P5A2 scFv, then FACS-sorted to enrich for CAR+ T cells. Sorted CAR+ T cells were then cultured in Grex-24 plates for a further 2 days in T cell expansion media, with CACCR CAR+ T cells left in the absence of exogenous cytokines, and with sorted control CAR+ T cells either left in the absence of exogenous cytokines, treated with 100 U/mL human IL-2, or treated with 10 ng/ml human IL-15. On Day 15, live CAR+ T cells were enriched using the Easy Sep Dead Cell Removal Kit (StemCell Technologies), and cell pellets were snap-frozen for subsequent RNA extraction and NanoString gene expression analysis (Human CAR-T Panel; NanoString Technologies).

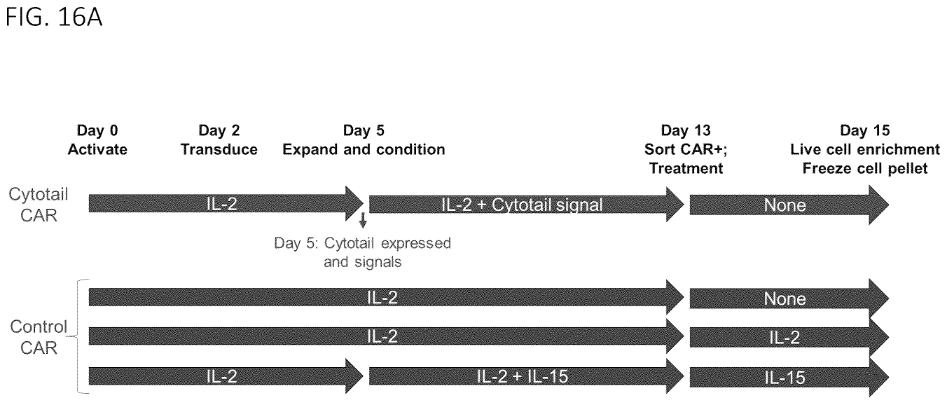

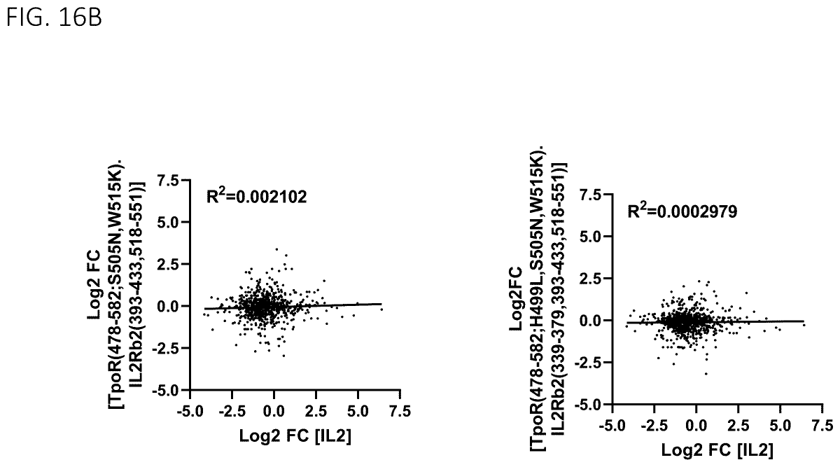

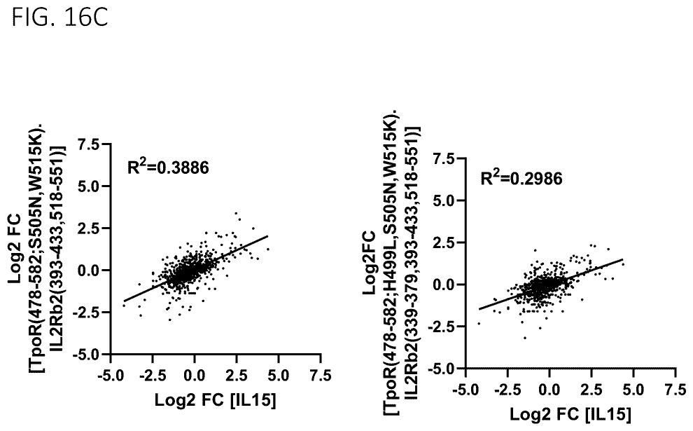

The data show that CACCR CAR-T cells bearing truncated IL2Rb tails more closely mimic IL-15, rather than IL-2, signaling. As an example, we tested the cytotails TpoR(478-582; S505N, W515K).IL2Rb(393-433,518-551) and TpoR(478-582; H499L,S505N, W515K). IL2Rb(339-379,393-433,518-551). FIG. 16A is a schematic diagram of the experimental design and workflow for sample preparation. FIG. 16B shows the gene expression profile of CACCR CAR-T cells compared to that of control CAR-T cells treated with IL-2 from Days 13-15. FIG. 16 C shows the gene expression profile of CACCR CAR-T cells compared to that of control CAR-T cells treated with IL-15 from Days 13-15. Log 2 fold change (FC) of each sample was calculated by normalization to control CAR-T cells that were left untreated from Days 13-15. The R² values and best-fit line (solid line) as determined by linear regression analysis are shown on each graph. Data shown is one representative of two donors. While the gene expression profiles of CACCR CAR-T cells showed no correlation with IL-2-treated samples (FIG. 16B), they correlated positively with IL-15-treated samples (FIG. 16C). These suggest that cytotails bearing the truncated IL2Rb tails more closely mimic the downstream signaling and transcriptional responses of IL-15, instead of IL-2.

### Example 9: Constitutive Cytotails can be Programmed for Combinatorial Signaling Outputs

As shown in FIG. 12, CACCRs bearing signaling domains derived from various cytokine receptors can activate signaling reminiscent of the parental receptor. We hypothesized that cytotails can be designed to mimic simultaneous signaling from multiple parental receptors by fusing more than one cytotail in tandem, allowing combinatorial signaling outcomes to be achieved. To test combinatorial signaling outputs from tandem cytotails, we generated a constitutive 7.12 tail by fusing the IL7Ra(316-459) cytotail with the truncated IL12Rb2(775-825) cytotail and evaluated signaling using the HEK293T cell reporter assay.

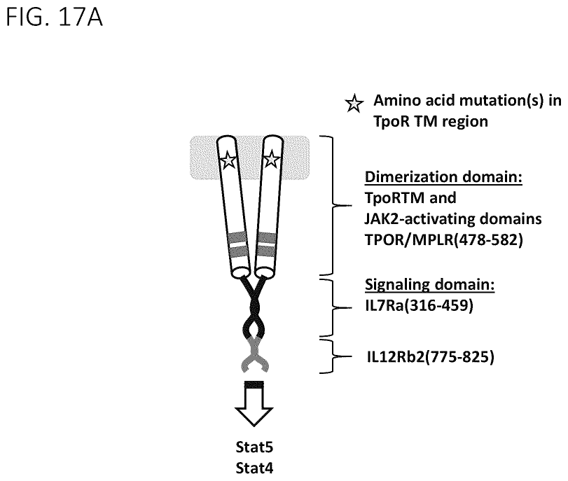

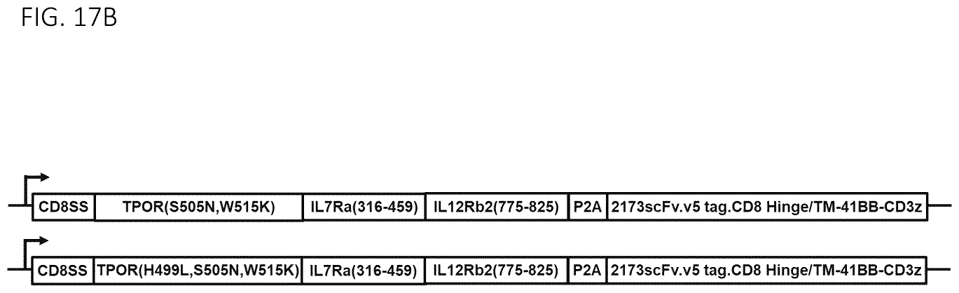

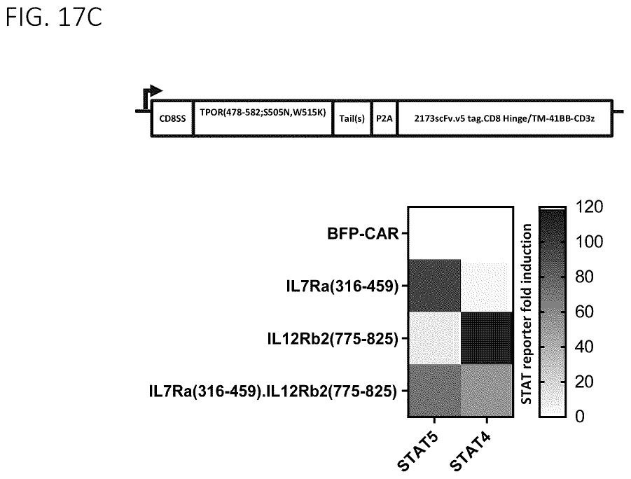

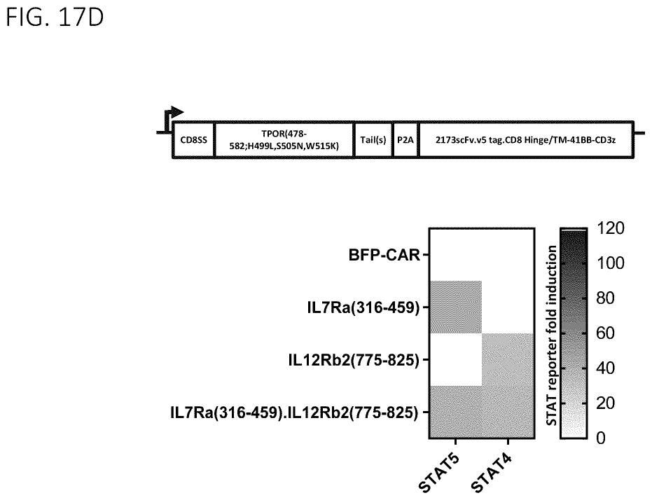

FIGS. 17A-D show the design and signaling capacity of constitutive tandem cytotails, as exemplified by the 7.12 tail. FIG. 17A shows a schematic of the constitutive 7.12 tail. FIG. 17B shows a schematic of the lentiviral vectors, differing only in their TpoR(478-582) dimerization/JAK-binding domains, used to co-express the 7.12 tail variants and a CAR. FIGS. 17C-D show STAT reporter activity for constructs bearing the TpoR(478-582; S505N; W515K) and TpoR(478-582; H499L; S505N; W515K) dimerization/JAK-binding domains, respectively, fused to the indicated cytotails. While the IL7Ra(316-459) cytotail strongly activated STAT5, the IL12Rb2(775-825) cytotail strongly activated STAT4. However, as observed in the IL7Ra(316-459). IL12Rb2(775-825) cytotail, fusing both cytotails in tandem resulted in simultaneous and combinatorial activation of both STAT5 and STAT4. This demonstrates that multiple signaling pathways that would usually require two or more distinct native cytokine receptors can be achieved with a single cytotail.

### Example 10: Constitutive Cytotails can be Tailored with Single or Multiple Outputs to Direct CAR-T Cell Phenotype and Function

We next determined if constitutive cytotails bearing different signaling outputs can differentially impact the phenotype and function of primary human CAR-T cells. Unlike IL-7 that drives T cell survival and memory maintenance, IL12 is a proinflammatory cytokine that promotes T cell differentiation. We therefore sought to interrogate if signaling through the IL7Ra- or IL12Rb-derived cytotails can differentially direct these divergent phenotypes, and to assess the net combinatorial effect of fusing both tails in tandem.

To this end, we generated human primary CAR-T cells that co-expressed either the 7 tail (i.e. IL7Ra(316-459)) or variants of 12 tails (i.e. IL12Rb2(775-825) or IL12Rb2(714-862)). See FIG. 13A. To make lentivirus encoding CACCR and CARs, HEK293T cells were plated at 0.45 million cells per mL in 2 mL of DMEM (Gibco) supplemented with 10% FBS (Hyclone) per well of a 6-well plate the day before transfection. On the day of transfection, the lentivirus was prepared by mixing together lentiviral packaging vectors 1.5 ug psPAX2, 0.5 ug pMD2G, and 0.5 ug of the appropriate transfer CAR vector in 250 uL Opti-MEM (Gibco) per well of the 6-well plate (“DNA mix”). 10 uL Lipofectamine 2000 (Invitrogen) in 250 uL Opti-MEM was incubated at room temperature for 5 minutes and then added to the DNA mix. The mixture was incubated at room temperature for 20 minutes and the total volume of 500 μL was slowly added to the sides of the wells containing HEK293T. 1 day post-transfection, the media from each well of HEK293T cells in the 6-well plate was replaced with 2 mL per well of T cell transduction media, i.e., X-Vivo-15 supplemented with 10% FBS. 2 days post-transfection, the lentiviral supernatants from HEK293T cells were harvested and passed through a 0.45 micron filter (EMD Millipore) to remove cell debris, and crude lentiviral supernatants were used directly for T cell transduction. On Day 0, purified T cells were activated in X-Vivo-15 medium (Lonza) supplemented with 100 IU/mL human IL-2 (Miltenyi Biotec), 10% FBS (Hyclone), and human T TransAct (Miltenyi Biotec, Cat #130-111-160, 1:100 dilution) in a Grex-24 plate (Wilson Wolf, cat #80192M). On Day2, T cells were resuspended at 0.5 million cells per mL in T cell transduction media, transduced with an equal volume of crude lentiviral supernatant along with 100 IU/mL human IL-2 in a Grex-24 plate. On Day 5, cells were fed by replacing the spent media with T cell expansion media, i.e., X-Vivo-15 supplemented with 5% human AB serum (Gemini Bio), along with 100 IU/mL human IL-2. Cells were expanded into larger G-Rex vessels (Wilson Wolf) as needed using T cell expansion media and the respective concentrations of human IL-2. On Day 14, memory phenotyping of the CAR-T cell products was performed by detecting CAR-transduced cells using a FITC-conjugated v5 tag monoclonal antibody (Thermo Fisher), together with co-staining with a PE/Cy7-conjugated CD62L antibody (Biolegend) and a BV785-conjugated CD45RO antibody (Biolegend) by flow cytometry. As negative controls that lacked CACCR signaling, CAR-T cells co-expressing BFP or the wildtype TpoR(478-582) transmembrane domain coupled to a 7 tail were generated in parallel.

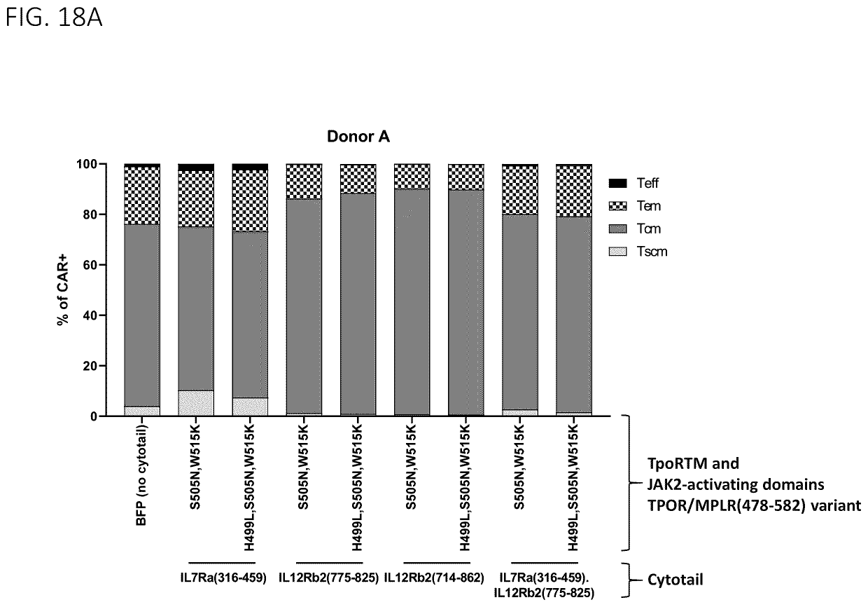

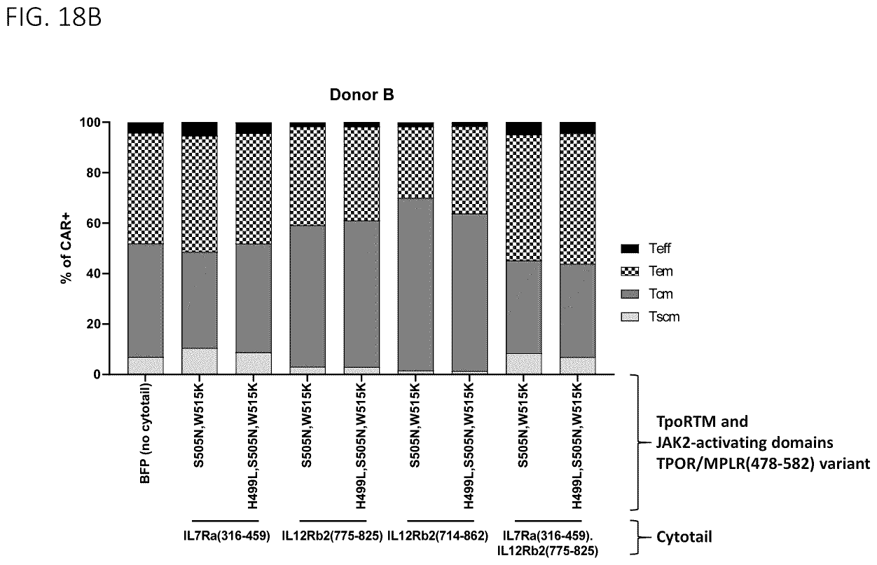

FIG. 18 depicts the impact of cytotails on memory differentiation of CAR-T cell products. The memory phenotype of Day 14 CACCR CAR-T cell products generated from 2 healthy donors is shown. While CAR-T cells co-expressing the IL7Ra(316-459) cytotail retained a stem cell memory (Tscm) population, CAR-T cells bearing IL12Rb2-derived cytotails showed marked reductions in the Tscm population accompanied by an increased central memory (Tcm) population. These suggested that in the absence of CAR engagement, constitutive IL12-like signaling through IL12Rb2-derived cytotails drive progressive differentiation from Tscm to Tcm. Furthermore, CAR-T cells co-expressing the tandem IL7Ra(316-459). IL12Rb2(775-825) cytotail showed a recovery of the Tscm population and mimicked the phenotype of CAR-T cells bearing the single IL7Ra(316-459) cytotail, indicating that negative effects of single cytotails can be mitigated through combinatorial signaling of tandem cytotails.

We further investigated if CACCR signaling could direct CAR-T cell functional outcomes, including survival and cytotoxicity. Compared to IL-7 signaling that promotes long-term T cell survival, IL-12 signaling instead drives differentiation into short-lived terminal effectors. In support of this, the Tscm population, which is capable of long-term survival and is believed to mediate prolonged CAR-T cell persistence, was scarce in CAR-T cells bearing IL12Rb2-derived tails. To interrogate if signaling through different cytotails could program CAR-T cell survival and differentiation, we performed a growth factor-independent assay in which CACCR coexpressing CAR-T cells were cultured in the absence of target cells or exogenously supplemented cytokines. Under these conditions, CAR-T cell numbers and memory differentiation were monitored over time.

Briefly, cryopreserved CAR-T cells were thawed, counted, and the percentage of CAR-T cells across all samples were normalized to the sample with the lowest transduction efficiency by the addition of non-transduced (NTD) T cells. As a control, CAR-T cells co-expressing BFP (BFP CAR) in place of a cytotail was used. 0.25×10⁶ CAR+ T cells/mL in 1.5 mL RPMI containing 10% FBS (Hyclone), non-essential amino acids, sodium pyruvate and 20-25 mM HEPES con were then seeded in 24-well tissue culture plates. On the indicated days, duplicate samples of 100 μL was harvested from each condition, and stained using the Zombie NIR Fixable Viability Kit (Biolegend). Samples were washed with PBS, Fc-blocked, then stained with the following antibody cocktail diluted in PBS+1% BSA: BUV395-conjugated anti-human CD3, BV510-conjugated anti-human CD8, BV605-conjugated human CD4 and FITC-conjugated v5 tag (for CAR detection), PE/Cy7-conjugated anti-human CD62L (Biolegend) and BV785-conjugated anti-human CD45RO (Biolegend). Finally, samples were washed in PBS and cell pellets were resuspended in 130 uL PBS+1% BSA containing 123count eBeads counting beads (Thermo Fisher) (10 uL counting beads in 120 uL PBS+1% BSA) prior to FACS analysis.

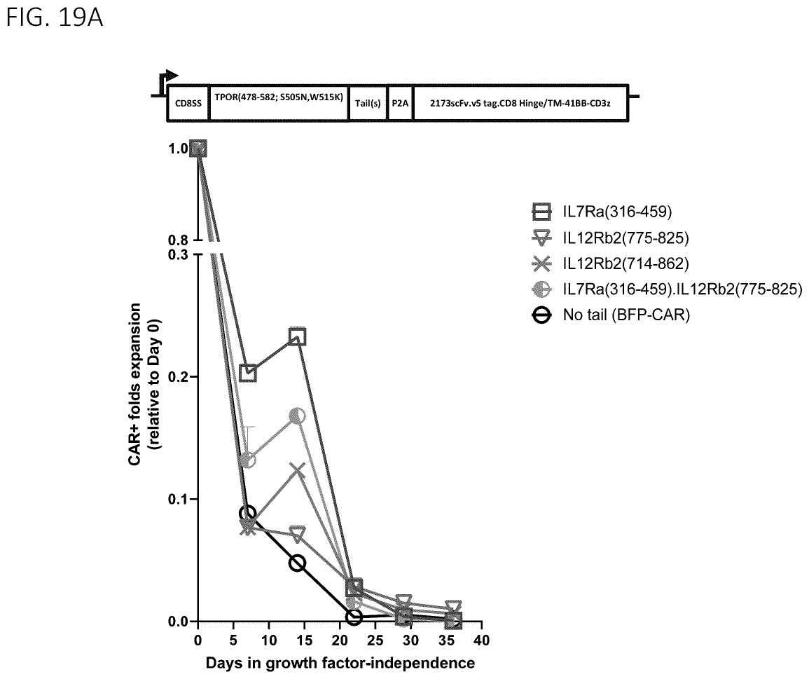

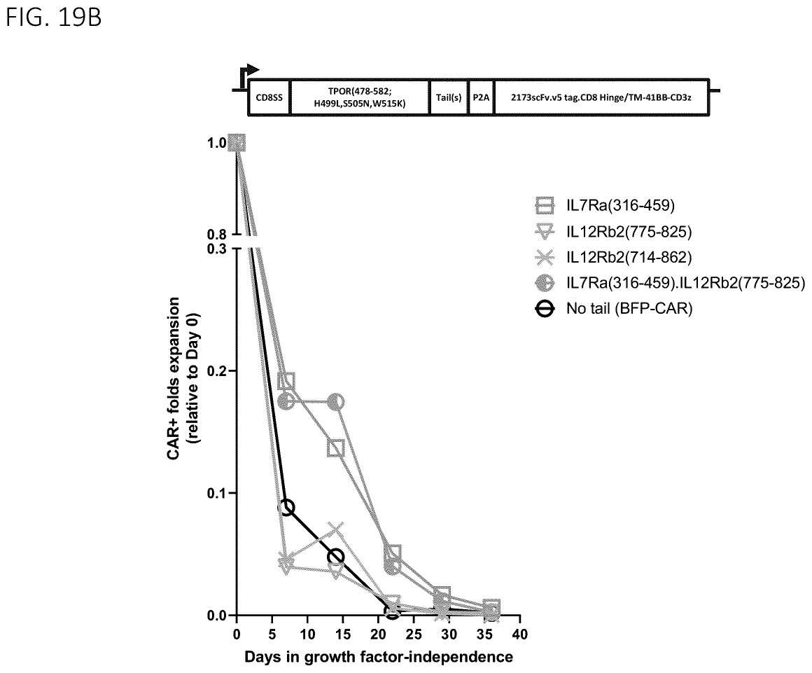

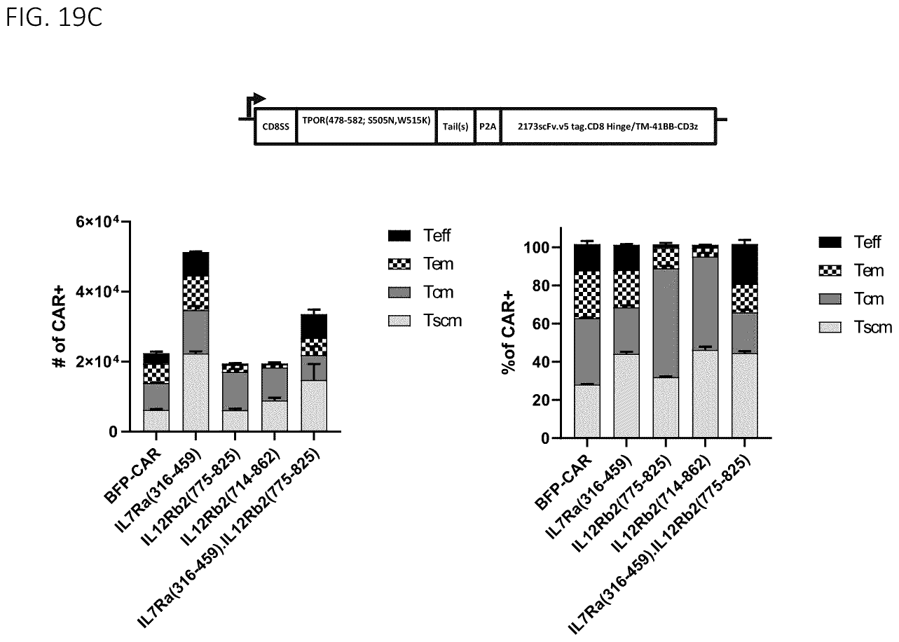

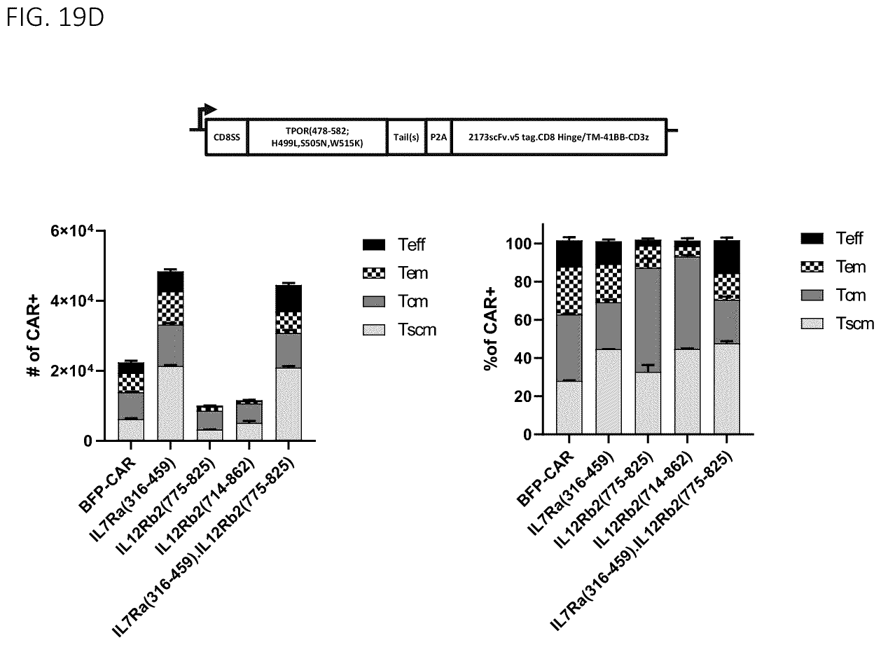

FIGS. 19A-B show representative data of cells from 2 donors. FIG. 19A and FIG. 19B show fold expansion of CAR-T cells relative to the input at the start of the assay (Day 0) for constructs bearing the TpoR(478-582; S505N; W515K) and TpoR(478-582; H499L; S505N; W515K) dimerization/JAK-binding domains, respectively, fused to the indicated cytotails. Compared to the control BFP CAR-T cells, CAR-T cells bearing the IL7Ra(316-459) cytotail declined at a slower rate, indicating that the constitutive signaling through the IL7Ra(316-459) cytotail improved CAR-T cells survival. In contrast, CAR-T cells bearing IL12Rb2-derived cytotails declined at a rate more comparable to BFP CAR-T cells, suggesting the lack of a survival benefit. Notably, CAR-T cells bearing the tandem IL7Ra(316-459). IL12Rb2(775-825) cytotail conferred a survival benefit more comparable to that of the IL7Ra(316-459) cytotail. FIGS. 19C-D show CAR-T cell differentiation on Day 7 of the growth factor-independent assay for constructs bearing the TpoR(478-582; S505N; W515K) and TpoR(478-582; H499L; S505N; W515K) dimerization/JAK-binding domains, respectively, fused to the indicated cytotails. Compared to control BFP CAR-T cells and CAR-T cells bearing IL12Rb2-derived cytotails, CAR-T cells bearing the IL7Ra(316-459) cytotail were not only more abundant, but were also more enriched in the Tscm population. See FIGS. 19C-D. CAR-T cells bearing the tandem IL7Ra(316-459) IL12Rb2(775-825) cytotail improved Tscm enrichment. Without being limited to any particular mechanisms, the results suggest that fusion with the IL7Ra(316-459) cytotail have overridden the phenotype of the single IL12Rb2(775-825) cytotail. Together, these data reiterate the combinatorial functional effects of tandem cytotail signaling and demonstrate tunability of different cytotail constructs.

CAR-T cell products enriched in the Tscm population are associated with improved expansion, persistence and activity. However, terminally-differentiated CAR-T cells are short-lived and have limited proliferative potential, leading to reduced efficacy. Given that these characteristics were differentially influenced by the IL7Ra(316-459) and IL12Rb2-derived cytotails, we evaluated the ability of these cytotails to impact CAR-T cell cytotoxicity.

To this end, 5,000 U87KO-EGFRvIII-nucGFP target cells were seeded and allowed to attach in 96-well plates with black walls and flat clear bottom in 50 uL RPMI containing 10% FBS (Hyclone), non-essential amino acids, sodium pyruvate and 20-25 mM HEPES. EGFRvIII CAR (2173 scFv) T cells bearing either the IL7Ra(316-459) or IL12Rb-derived cytotails were thawed and added to plated target cells at an E:T ratio of 1:3. Since target cells outnumber CAR-T cells, control of target cell growth would require CAR-T cells to kill repeatedly or serially. As a control, CAR-T cells co-expressing the BFP CAR in place of a cytotail was used. The number of live target cells over time was monitored via the IncuCyte Live Cell Analysis Imaging System.

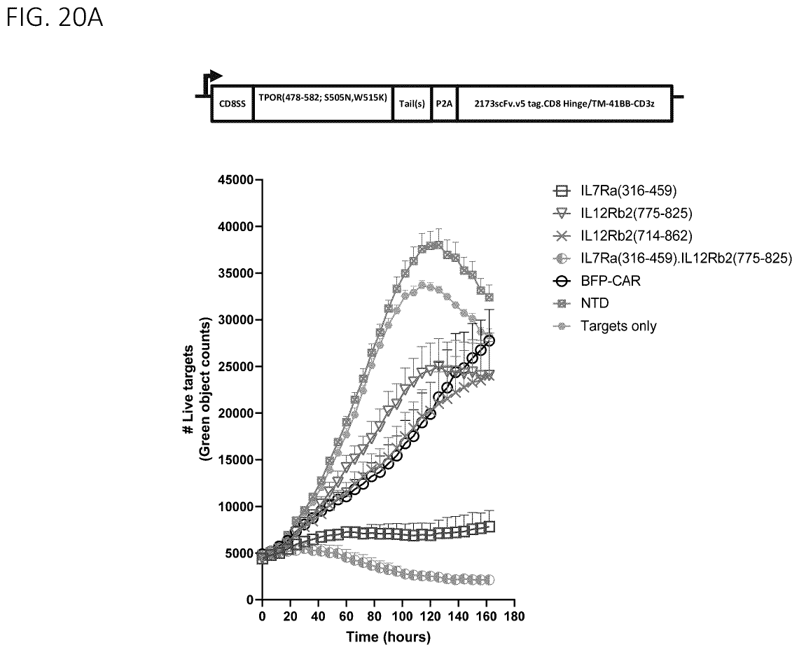

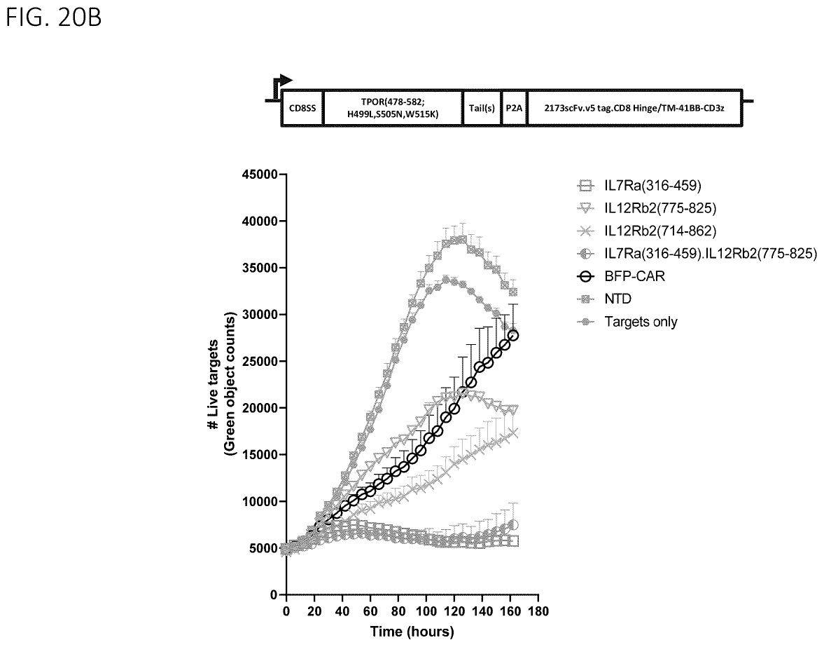

FIGS. 20A-B depict cytotoxic activity of CAR-T cells co-expressing various CACCRs. FIGS. 20A-B show target cell clearance by CACCR CAR-T cells bearing the TpoR(478-582; S505N; W515K) and TpoR(478-582; H499L; S505N; W515K) dimerization/JAK-binding domains, respectively, fused to the indicated cytotails. Compared to BFP CAR-T cells, CAR-T cells bearing IL12Rb2-derived cytotails showed some to no improvement in serial killing activity in vitro, likely due to their limited proliferative potential and short life-span. In contrast, CAR-T cells bearing the IL7Ra(316-459) cytotail exhibited improved serial killing activity, likely due to enhanced proliferation and persistence. Notably, CAR-T cells bearing the tandem IL7Ra(316-459). IL12Rb2(775-825) cytotail showed equal or better serial killing activity than the IL7Ra(316-459) cytotail, suggesting that combining the pro-persistence IL7Ra(316-459) signaling domain and the pro-effector IL12Rb2(775-825) signaling domain in a single cytotail may simultaneously enhance CAR-T cell longevity, expansion and immediate effector functions.

### Example 11: Constitutive Cytokine Receptors Enhance the In Vitro Cytotoxicity of CARs Directed Towards a Liquid Tumor Target

We have demonstrated that CACCRs can enhance the activity of a CAR directed towards EGFRvIII, a target for solid tumor, e.g., glioblastoma. To determine if CACCRs are broadly applicable across a different scFv for a hematological tumor target, we additionally cloned the CACCRs into CAR construct directed towards a marker for a hematological malignancy (i.e. BCMA) and evaluated the long-term cytotoxicity against the BCMA positive target cell line.

Target cells stably expressing the firefly luciferase and GFP reporters were generated by lentiviral transduction. 10,000 Luc-GFP-labelled target cells were plated in 100 uL per well in a white flat-bottomed 96-well tissue culture plate. Cryopreserved CAR-T cells were thawed, counted, and the percentage of CAR-T cells across all samples were normalized to the sample with the lowest transduction efficiency by the addition of non-transduced (NTD) T cells. CAR-T cells in a volume of 100 uL were then added to each well of target cells at the indicated Effector: Target (E: T) ratios in triplicates. As a “Targets only” negative control, 100 μL of media, instead of T cells, was added to target cells. After two or three days, wells were mixed by gentle pipetting, and 100 μL of each T cell-containing well was transferred to a new white flat-bottomed 96-well tissue culture plate containing 10,000 freshly-plated Luc-GFP-labelled target cells in 100 uL. “Targets only” wells received fresh media in place of T cells. The new plate was incubated at 37° C., while the number of live target cells remaining in the old 96-well plate was determined using the ONE-Glo Luciferase Assay System (Promega) according to manufacturer's instructions. The percentage of live target cells was calculated by normalizing the luciferase signal of to that of “Targets only” wells, and percentage cytotoxicity was calculated as 100%-% live target cells. Serial transfers to fresh target cells and luciferase readouts were performed every two or three days until all cytotoxic activity has ceased.

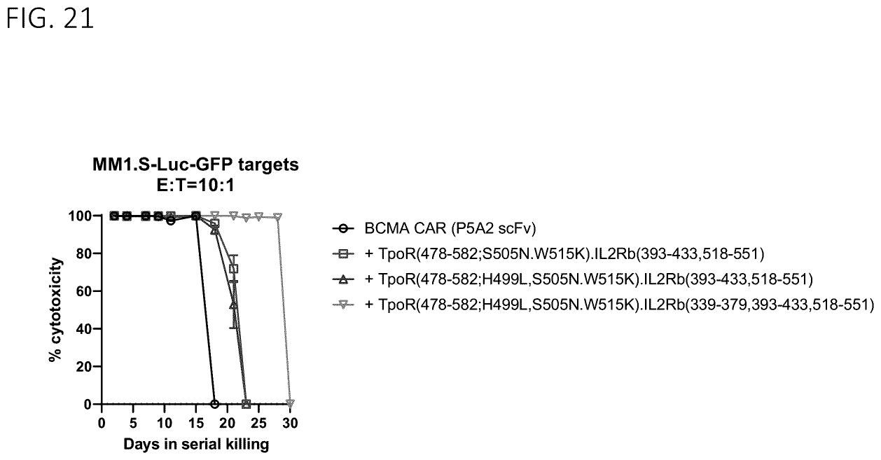

FIG. 21 shows that CACCRs improved the cytotoxic activity of CAR-T cells directed towards BCMA, a liquid tumor target. FIG. 21 shows the cytotoxicity of a BCMA CAR (P5A2 scFv) against the MM1.S multiple myeloma cell line at an E:T=10:1, indicating co-expression of a CACCR increased the long-term cytotoxicity of CAR-T cells.

### Example 12: CACCRs Enhance the In Vivo Activity of CAR-T Cells

CAR-T cell therapies, such as those targeting CD19 and BCMA, have attained unprecedented clinical success in the treatment of hematological malignancies. While a high rate of complete responses has been achieved, this is transient as most patients eventually relapse. Furthermore, CAR-T cells have attained more limited success for the treatment of solid tumors. Among the reasons for relapse and the lack of response include insufficient CAR-T cell expansion and persistence, as well as CAR-T cell functional inhibition by immune-suppressive microenvironments. Since our in vitro characterization of CACCR CAR-T cells revealed improvements in target-driven proliferation, persistence, potency and exhaustion profiles, we next investigated whether these functional enhancements translated into improved anti-tumor activity in vivo.

To interrogate the in vivo activity of CACCR CAR-T cells in the context of hematological malignancies, we utilized CAR-T cells bearing the BCMA-specific P5A2 scFv coupled to 4-1BB and CD3ζ signaling domains in an orthotopic xenograft model of multiple myeloma. T cell receptor (TCR)-deficient BCMA CAR-T cells were generated by Transcription Activator-Like Effector Nucleases (TALEN)-mediated knockout to avoid potential confoundance from TCR-driven xenoreactivity. 8-10 week old female NSG mice were irradiated with 1 Gy one day prior to intravenous inoculation of 5×10⁶ MM1.S-Luc-GFP. 14 days after tumor implantation, mice were randomized based on tumor burden, and dosed intravenously with either 1×10⁶ or 3×10⁶ of the indicated CAR-T cells (n=10 per group). Tumor progression was monitored by bioluminescent imaging. On Day 30 post T cell dose, mice that had received 3×10⁶ CAR-T cells were bled for the enumeration of BCMA CAR-T cells in the periphery. Specifically, 50 μL of whole blood from each mouse was subjected to red blood cell lysis using ACK Lysing Buffer (Gibco), Fc-blocked and stained with the following antibody cocktail diluted in PBS+1% BSA: FITC-conjugated anti-mouse CD45 (Biolegend), BV421-conjugated anti-human CD45 (Biolegend) and an anti-idiotype antibody specific for the P5A2 scFv. Finally, samples were washed in PBS and cell pellets were resuspended in 130 uL PBS+1% BSA containing 123count eBeads counting beads (Thermo Fisher) (10 uL counting beads in 120 uL PBS+1% BSA) prior to FACS analysis.

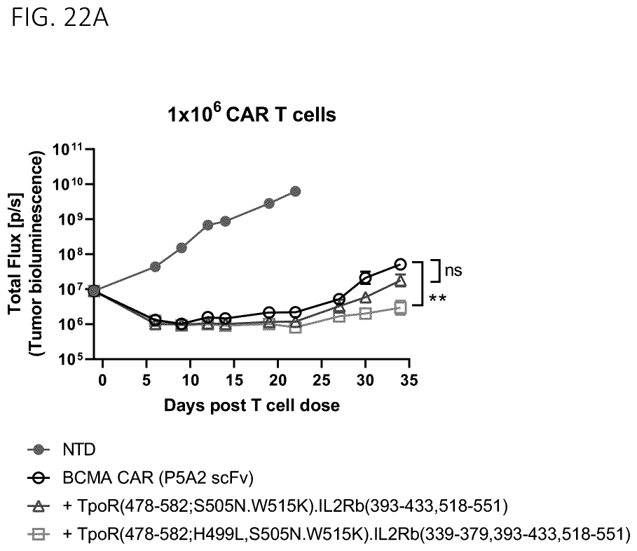

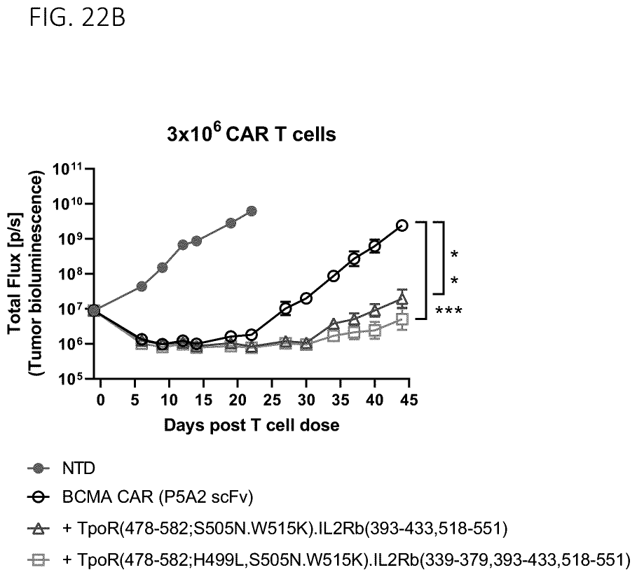

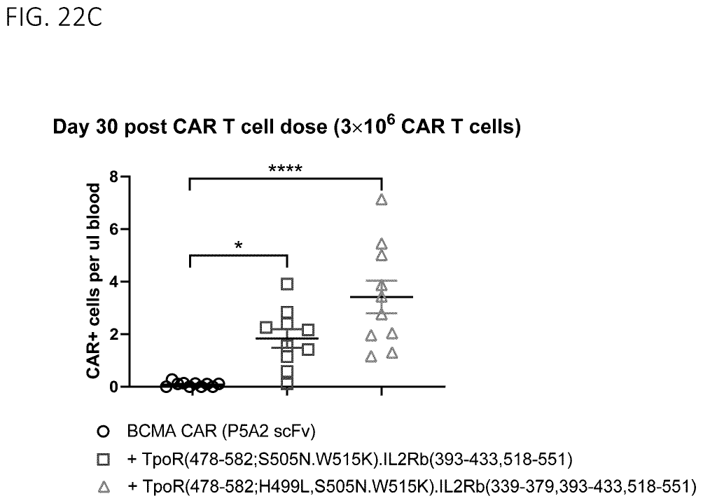

FIGS. 22A-C show that CACCRs improved the in vivo anti-tumor activity and persistence of BCMA CAR-T cells against orthotopic multiple myeloma. FIGS. 24A-B show tumor progression in response to treatment with either 1×10⁶ or 3×10⁶ of the indicated CAR-T cells, respectively. Although control BCMA CAR-T cells were able to mediate initial tumor regression, this response was short-lived as tumors relapsed 22 days after cell infusion. However, CACCR coexpressing CAR-T cells significantly delayed tumor relapse and improved the durability of response. Statistics in FIGS. 24A-B represent ** p<0.01 and *** p<0.001 based on repeated measures one-way ANOVA with Tukey's multiple comparisons from Days 6-34 for FIG. 22A and Days 6-44 for FIG. 22B. FIG. 22C shows the number of BCMA CAR-T cells present in the peripheral blood of mice treated with 3×10⁶ CAR-T cells 30 days after T cell infusion. Coincident with tumor relapse observed in mice treated with control BCMA CAR-T cells, control BCMA CAR-T cells could no longer be detected in the periphery. In contrast, CACCR BCMA CAR-T cells that were superior at preventing tumor relapse were also more significantly abundant in vivo. Statistics in FIG. 24C represent *p<0.05 and **** p<0.0001 based on ordinary one-way ANOVA with Tukey's multiple comparisons. These suggest that improved CACCR CAR-T cell persistence in part mediated enhanced long-term tumor control and prolonged the durability of response.

We additionally assessed the impact on CACCRs on CAR-T cell activity in the context of solid tumors known to resist CAR-T cell therapy, such as glioblastoma. To this end, we utilized a EGFRvIII-specific CAR bearing the 2173 scFv coupled to 4-1BB and CD3ζ signaling domains, as well as the LN229 human glioblastoma cell line stably over-expressing EGFRvIII (LN229-EGFRvIII). 8-10 week old female NSG mice were subcutaneously implanted with 3×10⁶ LN229-EGFRvIII. 25 days later when tumors were established, mice were randomized based on tumor burden and dosed intravenously with either 1.5×10⁶ or 3×10⁶ of the indicated CAR-T cells (n=8-10 per group). Tumor progression was monitored twice a week by caliper measurements.

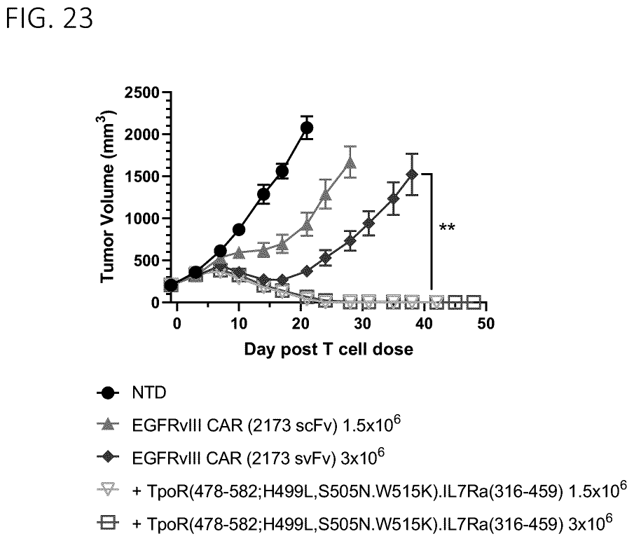

FIG. 23 shows that CACCR improved the anti-tumor activity of CAR-T cells against established solid tumors. Although treatment with control EGFRvIII CAR-T cells could retard the growth of LN229-EGFRvIII tumors, the response was transient and sub-optimal as tumors eventually progressed. In contrast, treatment with CACCR CAR-T cells resulted in complete tumor regression. Notably, even a low dose of 1.5×10⁶ CACCR CAR-T cells was sufficient for tumor elimination. Statistics represent ** p<0.01 compared to treatment with 3×10⁶ control CAR-T cells based on repeated measures one-way ANOVA with Tukey's multiple comparisons from Days 3-38. These results reiterate the ability of CACCRs to synergize non-redundantly with signaling domains in CAR-T cells to confer improved activity.

1-57. (canceled)

58-85. (canceled)

86. A chimeric cytokine receptor (CACCR) comprising a monomer, wherein the monomer comprises
a) a TpoR transmembrane domain; and
b) a STAT-activation recruiting domain which comprises an IL2Rb amino acid sequence comprising SEQ ID NO: 77 or 78.

87. The CACCR of claim 86, wherein the STAT-activation recruiting domain further comprises an IL21R amino acid sequence comprising SEQ ID NO: 54.

88. The CACCR of claim 86, wherein the STAT-activation recruiting domain further comprises an IL12Rb1 amino acid sequence comprising SEQ ID NO: 86 or 87.

89. The CACCR of claim 86, wherein the STAT-activation recruiting domain further comprises an IL12Rb2 amino acid sequence comprising SEQ ID NO: 67.

90. The CACCR of claim 86, wherein the STAT-activation recruiting domain further comprises an IL7Ra amino acid sequence comprising SEQ ID NO: 68, 69, 70, 71, or 72.

91. The CACCR of claim 86, wherein the TpoR transmembrane domain comprises amino acids 478-582 of SEQ ID NO: 6 and comprises amino acid substitutions S505N and W515K.

92. The CACCR of claim 91, wherein amino acids 478-582 of SEQ ID NO: 6 further comprise an amino acid substitution H499L.

93. The CACCR of claim 91, wherein amino acids 478-582 of SEQ ID NO: 6 further comprise an amino acid substitution H499L and G509N.

94. The CACCR of claim 86, which is composed of two monomers.

95. The CACCR of claim 94, wherein the two monomers are identical.

96. The CACCR of claim 94, wherein the two monomers are different.

97. A polynucleotide encoding the CACCR of claim 86.

98. An expression vector comprising the polynucleotide of claim 97.

99. The expression vector of claim 98 further comprising a polynucleotide expressing a chimeric antigen receptor (CAR).

100. The expression vector of claim 98, wherein the vector is a lentiviral vector.

101. An engineered immune cell comprising the expression vector of claim 98.

102. The engineered immune cell of claim 101, wherein the immune cell is a T-cell.

103. An engineered immune cell comprising a chimeric antigen receptor (CAR) and at least one CACCR of claim 86.

104. The engineered immune cell of claim 103, wherein the CAR and the CACCR are expressed in stoichiometrically equal amounts.

105. The engineered immune cell of claim 103, wherein the immune cell is a T-cell.

106. A method of preparing an engineered immune cell, the method comprising introducing the polynucleotide of claim 97 into an immune cell.

107. A pharmaceutical composition comprising the engineered immune cell of claim 101.

108. A kit comprising the engineered immune cell of claim 101.

109. A method of treating a condition in a subject, comprising administering to the subject a therapeutically effective amount of the engineered immune cell of claim 101.

110. The method of claim 109, wherein the condition is cancer, an autoimmune disorder, or an infection.

111. The method of claim 110, wherein the cancer comprises a solid tumor.

112. The method of claim 110, wherein the cancer comprises a liquid tumor.
